(19) United States
(12) Patent Application Publication
(10) Pub. No.: US 2022/0281902 A1
(43) Pub. Date: Sep. 8, 2022

(54) MITOCHONDRIAL MODULATION TO IMPROVE METABOLIC SYNDROME DURING AGING

(71) Applicants: The Board of Trustees of the Leland Stanford Junior University, Stanford, CA (US); Shanghai Jiao Tong University, Shanghai (CN)

(72) Inventors: Lei Fu, Shanghai (CN); Mojdeh Tavallaie, Shanghai (CN); James P. Collman, Stanford, CA (US); Christopher J. Barile, Stanford, CA (US); Yixin Hu, Shanghai (CN)

(21) Appl. No.: 17/636,003

(22) PCT Filed: Aug. 11, 2020
(86) PCT No.: PCT/US2020/045787

§ 371 Date: Feb. 16, 2022

Related U.S. Application Data
(60) Provisional application No. 62/888,921, filed on Aug. 19, 2019.

(51) Int. Cl. C07F 9/6539; A61P 3/10; A61P 3/04; C07D 277/26; C07D 277/28; C07F 9/653; C07D 417/12; C07F 9/6518

(52) U.S. Cl. CPC C07F 9/6539; A61P 3/10; A61P 3/04; C07D 277/26; C07D 277/28; C07F 9/65318; C07D 417/12; C07F 9/6518

(57) ABSTRACT

Compounds, compositions and methods are provided for mitochondrial modulation. The subject mitochondrial modulator compounds generally include a head group linked to a charged moiety. In certain cases, the head group is a heterocyclic or a heteroaryl group. Aspects of the subject methods include a method of modulating mitochondria. Aspects of the subject methods include treating a subject having a metabolic syndrome-related disease or a symptom thereof by administering to the subject a therapeutically effective amount of a subject compound. In certain cases, the disease is selected from hyperlipidemia, type 2 diabetes, fatty liver disease, obesity, cardiovascular disease and stroke. In certain cases, the symptom is selected from abdominal obesity, insulin resistance, hyperinsulinemia, high levels of blood fats, increased blood pressure, and elevated serum lipids.

### CROSS-REFERENCING

This application claims the benefit of U.S. provisional application Ser. No. 62/888,921, filed on Aug. 19, 2019, which application is incorporated by reference herein.

### INTRODUCTION

Many of the hallmarks of aging can be traced to the degradation of mitochondrial health and efficiency. Decline in mitochondrial operation and the accumulation of abnormal mitochondria can lead to metabolic disorders. Metabolic syndrome is an associated cluster of traits that includes, but is not limited to, hyperinsulinemia, abnormal glucose tolerance, obesity, redistribution of fat to the abdominal or upper body compartment, hypertension, dysfibrinolysis, and dyslipidemia characterized by high triglycerides, low high density lipoprotein (HDL)-cholesterol, and high small dense low density lipoprotein (LDL) particles. Subjects having metabolic syndrome are at risk for development of Type 2 diabetes and/or other disorders (e.g., atherosclerosis).

Mitochondria are organized inside cells to form an interconnected and dynamic network, regulated by mitochondrial dynamics. Alteration of mitochondrial dynamics in ageing could explain the accumulation of mitochondrial damage and be viewed as a mechanism linking a loss of mitochondrial fitness with a causative role in the pathogenesis of metabolic syndrome of ageing (Sebastian et al., Trends in Molecular Medicine. (2017), 23:3, p. 201-215).

Weight loss, exercise, a healthy diet and refraining from smoking is advised for preventing and treating metabolic syndrome. Nutritionally balanced diet with calorie restriction (CR) is also advised to delay the onset of age-associated pathologies and to promote a healthier and longer life in most organisms.

Curing obesity, diabetes and pre-diabetic irregularities are priorities to promote healthy ageing and metabolic syndrome alleviation. Although, calorie restriction and exercise are the first line of treatment, pharmacological treatment for spontaneous type 2 diabetes and obesity can be an effective method too, for example metformin. Metformin is an anti-diabetic drug which mimics the beneficial effects of calorie restriction by activating AMP-activated kinase (AMPK); a documented method of slowing and reversing biomarkers of human ageing including obesity and insulin resistance (Choi et al., Mol. Cells., (2013), 36:4, pp. 279-287).

In view of the prevalence and severity of obesity, diabetes and associated metabolic disorders, alternative agents and methods that may recapitulate the effects of CR, hinder the process of mitochondrial decay (mito-decay) and along with it, the course of ageing and metabolic syndrome are of interest.

### SUMMARY

Compounds, compositions and methods are provided for the mitochondrial modulation. The subject mitochondrial modulator compounds generally include a head group linked to a charged moiety. In certain cases, the head group is a heterocyclic or a heteroaryl group. Aspects of the subject methods include a method of modulating mitochondria, (e.g., moderating or inhibiting mitochondria) Aspects of the subject methods include treating a subject having a metabolic syndrome-related disease or a symptom thereof by administering to the subject a therapeutically effective amount of a subject compound. In certain cases, the disease is selected from hyperlipidemia, type 2 diabetes, fatty liver disease, obesity, cardiovascular disease and stroke. In certain cases, the symptom is selected from abdominal obesity, insulin resistance, hyperinsulinemia, high levels of blood fats, increased blood pressure, and elevated serum lipids.

### BRIEF DESCRIPTION OF THE FIGURES

FIG. 1A provides CcO activity measurements for 2 month and 14 month old mice vs control groups (n=3-5 per group).

FIG. 1B provides ATP production assessment in two groups of 6 and 14 month old mice, treated vs. untreated controls (n=3 per group).

FIG. 1C provides comparisons of single vs. long term treatment in mice at ages of 2 and 14 months old (n=3-5).

FIG. 1D, provides qPCR microarrays of mitochondriogenesis transcripts and mitophagy markers in livers of 12 months and 16 months old treated and control (untreated) mice after 10 and 14 months long treatment (n=3-5 per genotype).

FIG. 1E, provides qPCR microarrays of mitochondriogenesis transcripts and mitophagy markers in WAT[F] and hearts[H] of 16 months old mice after 14 months treatment (n=3-5 per genotype).

FIG. 1F, panels A-D, provides CcO activity measurements and analyses of whole mitochondrial respiratory units' levels utilizing mitochondrial OXOPHOS cocktail and mitochondrial complex IV antibody, all assessed by immunoblotting of 16 month old mice livers (n=5) and compared with the controls (untreated).

FIG. 2A-2B, illustrate analyses of body weight (BW) variations in treated and control groups of male mice (14 months) and female mice (10 months) during a 14 month long chow diet (CD), n=6-10 per group.

FIG. 2C-2D, illustrate analyses of BW variations in treated and control groups of male mice (12 months and 12 weeks old) during an 8 week long high fat diet (HFD, 60% fat), n=6 per group.

FIG. 2E, depicts a comparison graph of CD fed and HFD fed, 8 week long treated mice (14 months old) vs. controls, n=6 per group.

FIG. 2F, depicts BW, fat mass and lean mass analyses be BEXA body composition analysis in 14 month old mice (treated vs. control), n=2-3 per group.

FIG. 2G, depicts percentile of brown adipose tissue and visceral white adipose tissue in 14 month old mice (treated vs. control), n=3 per group.

FIG. 2H, illustrates qPCR microarrays of thermoregulatory factors and beiging inducers after 14 months of treatment vs. a control.

FIG. 3A, illustrates visceral fat tissue, as assessed by Toluidine Blue O (TBO) staining, taken from 5 and 12 month old control and treated mice, n=3 per group.

FIG. 3B, illustrates analysis of white adipocyte areas and perimeters.

FIG. 3C, illustrates qPCR microarrays of adipogenesis regulators and white fat tissue inducing transcripts in visceral fat after 14 months of treatment vs. a control, n=4 per genotype.

FIG. 3D, depicts treated and control 16 month old mice models: Heftier abdominal section and gray hairs are significant in control vs treatment. Visceral white fat accumulation in control model is greater than that of treated mice, and the difference in color of adipose tissue in treated and control is evident (dissection image, left bottom two panels), n=3.

FIG. 4A, illustrates in vitro adipocytes differentiation using oil red 0 staining method, adipogenesis evaluation and protein blotting assessment.

FIG. 4B, provides qPCR microarrays of inflammatory factors, macrophage pan marker and leptin receptor in treated and untreated (control) mice after 14 months treatment (n=4 per genotype).

FIG. 4C, provides serum ROS generation evaluation in mice after 14 months of treatment (n=6 per group).

FIG. 4D, provides serum ROS generation evaluation in 18 m old mice after 16 months of treatment, compared to 18 m-calorie reduction (CR) model and same age control (n=6 per group).

FIG. 4E, illustrates qPCR microarrays of DNA damage and WAT senescence markers in treated and untreated mice after 14 months treatment (n=3 per genotype).

FIG. 4F, illustrates a pro-inflammatory protein levels assay (n=3 per genotype).

FIG. 4G, provides mitochondrial membrane potential (Δψm) signal assay by using 5,5,6,6-tetrachloro-1,1,3,3-tetraethylbenzimidalolylcarbocyanine iodide (JC1) in 12-month-old mouse livers (n=4).

FIG. 5A, left panel illustrates blood glucose levels measured at the indicated times for weight-matched mice after 12-16 hr fast (n=10). Areas under the curves (AUCs) and comparison of young and old age group are shown in the right panel.

FIG. 5B, left panel illustrates blood insulin levels measured at the indicated times for weight-matched mice after 12-16 hr fast (n=10). Areas under the curves (AUCs) and comparison of young and old age group are shown in the right panel.

FIG. 5C, left panel illustrates plasma triglyceride levels measured after an overnight fast from animals in each group at the indicated times (n=5 per group). Areas under the curves (AUCs) and comparison of young and old group are shown in the right panel.

FIG. 5D, left panel illustrates plasma concentrations of cholesterol measured at indicated times of treatment, in mice after an over-night fast (n=5 per group). Areas under the curves (AUCs) and comparison of young and old group are shown in the right panel.

FIG. 5E-FIG. 5I, illustrate intra-peritoneal glucose tolerance test (IPGTT) at 2, 3, 6, 12- and 18-month old mice during treatment, and AUCs comparisons (n=10). All values are presented as mean±SEM.

FIG. 6A, illustrates quantitative polymerase chain reaction (qPCR) microarrays of hepatic glucose metabolism transcripts and aging phenotype markers in treated and untreated (control) mice livers after 14 months treatment (n=4 per genotype).

FIG. 6B, illustrates an immunoblotting assays of hepatic glucose metabolism and longevity phenotype markers in treated and controls (untreated) mice livers after 14 months treatment (n=3-4 per genotype).

FIG. 6C, illustrates chronic 10-week long treatment in 12 week old T2DM mice (n=3 per group).

FIG. 6D, illustrates blood insulin levels during 10 weeks chronic treatment of T2DM mice compared with healthy controls (n=3 per group).

FIG. 6E, illustrates glucose uptake after insulin (10 μM) induction.

FIG. 6F, illustrates 100% specific absorbance of 2-NDBG uptake among different concentrations of subject compound and control and positive control (Rosiglitazone) in mature 3T3-L1 cells.

FIG. 7, provides a comparison between CR and mitochondrial respiration moderation by CcO moderate inhibition and their major bio-cellular impacts.

FIG. 8, illustrates body temperature monitoring in C57BL/6 male mice after drug administration (3 different doses) over a period of 80 minutes.

FIG. 9, provides MTT assay and toxicity assay of an exemplary compound at various doses.

FIG. 10, provides LCMS analysis of subject compound concentration in various organs at 30 minutes and 1 hour.

FIG. 11, shows the qPCR primers used in the RT-qPCR experiments described herein. SEQ ID NOS: 1-56.

FIG. 12, panels A-E depicts images of the in vivo cervical cancer mouse study. Panel A depicts a saline control mouse. Panel B depicts a Taxol control mouse. Panel C-E depict mice dosed with 5 mg/kg, 20 mg/kg and 50 mg/kg of compound HG1a-1 respectively.

FIG. 13, illustrates the tumor weight measured in the in vivo cervical cancer mouse study after 10 days of administering compound HG1a-1.

FIG. 14, illustrates the tumor volume measured in the in vivo cervical cancer mouse study after 10 days of administering compound HG1a-1.

All values are presented in the accompanying figures as mean±SEM. Asterisks indicate statistical significances compared to controls using one-way ANOVA (*p<0.05; **p<0.01; ***p<0.001; ****p<0.0001).

### Definitions

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112. In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein the term “modulating mitochondria” refers to the modulation (e.g., moderation or inhibition) of mitochondria. In some cases, modulating mitochondria may includes inhibiting cytochrome c oxidase (CcO), e.g., CcO Complex IV of mitochondrial respiratory chain. In certain cases, modulating mitochondria may include inhibiting a mitochondrial Complex III of mitochondrial respiratory chain (cytochrome b_{c1} complex). In certain cases, modulating mitochondria may include inhibiting a mitochondrial Complex II of mitochondrial respiratory chain (succinate dehydrogenase). In certain cases, modulating mitochondria may include inhibiting a mitochondrial Complex I of mitochondrial respiratory chain (NADH dehydrogenase).

As used herein, the phrases “for example,” “for instance,” “such as,” or “including” are meant to introduce examples that further clarify more general subject matter. These examples are provided only as an aid for understanding the disclosure and are not meant to be limiting in any fashion.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action. The term “metabolic syndrome” is a term that is understood in the art, and refers to metabolic abnormalities, including central obesity, insulin resistance, hyperlipidemia, hyperglycemia, hypertension, and hepatic steatosis. The International Diabetes Foundation definition of metabolic syndrome is central obesity (body mass index>30 kg/m2) and two or more of: 1) triglycerides >150 mg/dL; 2) high density lipoprotein (HDL) <40 mg/kL in males, <50 mg/dL in females, or specific treatment for low HDL; 3) elevated blood pressure, e.g., systolic BP >130 mm Hg or diastolic BP >85 mm Hg, or treatment for elevated BP, or previous diagnosis of elevated BP; and 4) fasting blood glucose >100 mg/dL or previous diagnosis of type 2 diabetes.

The term “metabolic syndrome” refers to an associated cluster of traits that includes, but is not limited to, hyperinsulinemia, abnormal glucose tolerance, obesity, redistribution of fat to the abdominal or upper body compartment, hypertension, dysfibrinolysis, and dyslipidemia characterized by high triglycerides, low high density lipoprotein (HDL)-cholesterol, and high small dense low density lipoprotein (LDL) particles. Subjects having metabolic syndrome are at risk for development of Type 2 diabetes and/or other disorders (e.g., atherosclerosis).

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

As used herein, the term “charged group” with reference to a “charged group” on a subject compound that includes both a “charged side group” on a substituent comprised within the group referred to herein as “X” or “X⁴” on any of formulae (I)-(IE), and a “charged end group” on the group “X” or “X⁴” within the subject compound. It will be understood that the charge of a compound will in general be affected by the ambient medium. Thus, the term “charged group” herein refers to a group that is charged when the compound that comprises it is placed in water at 25° C. and having a pH of 7.4. Examples of typical charged groups include ammonium, carboxylate, guanidinium, phosphonium, pyridinium, imidazolium, sulfate and phosphate.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like.

“Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from the group consisting of hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

“Carboxyl,” “carboxy” or “carboxylate” refers to —CO₂H or salts thereof.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO— heteroaryl, —SO₂-alkyl, —SO₂-substituted alkyl, —SO₂-aryl and —SO₂-heteroaryl, and trihalomethyl.

As used herein, the terms “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Hydrocarbyl” refers to univalent hydrocarbyl radicals containing 1 to about 30 carbon atoms, including 1 to about 24 carbon atoms, further including 1 to about 18 carbon atoms, and further including about 1 to 12 carbon atoms, including linear, branched, cyclic, saturated and unsaturated species, such as alkyl groups, alkenyl groups, aryl groups, and the like. A hydrocarbyl may be substituted with one or more substituent groups. The term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH2), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH2), carbamido (—NH—(CO)—NH2), cyano (—C≡N), isocyano (—N+≡C—), cyanato (—O—C≡N), isocyanato (—O—N+≡C—), isothiocyanato (—S—CEN), azido (—N═N+═N—), formyl (—(CO)—H), thioformyl (—(CS)—H), amino (—NH2), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO2), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphine (—PH₂), mono- and di-(C1-C24 alkyl)-substituted phosphine, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

By “linking” or “linker” as in “linking group,” “linker moiety,” etc., is meant a bivalent radical moiety that connects two groups via covalent bonds. Examples of such linking groups include alkylene, alkenylene, alkynylene, arylene, alkarylene, aralkylene, and linking moieties containing functional groups including, without limitation: amide (—NH—CO—), ureylene (—NH—CO—NH—), imide (—CO—NH—CO—), epoxy (—O—), epithio (—S—), epidioxy (—O—O—), carbonyldioxy (—O—CO—O—), alkyldioxy (—O—(CH2)n-O—), epoxyimino (—O—NH—), epimino (—NH—), carbonyl (—CO—), thiocarbonyl (—CS—) etc. Any convenient orientation and/or connections of the linkers to the linked groups may be used.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻ M⁺, —OSO₂OR⁷⁰, —P(O)(O)⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as +N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M+, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

Definitions of other terms and concepts appear throughout the detailed description.

### DETAILED DESCRIPTION

Compounds, compositions and methods are provided for the mitochondrial modulation. The subject mitochondrial modulator compounds generally include a head group linked to a charged moiety. In certain cases, the head group is a heterocyclic or a heteroaryl group. In certain cases, the head group is selected from a thiazole, an oxadiazole, a tetrazole, a triazine, and a guanidine. Aspects of the subject methods include a method of modulating mitochondria (e.g., inhibiting mitochondria). Aspects of the subject methods include treating a subject having a metabolic syndrome-related disease or a symptom thereof by administering to the subject a therapeutically effective amount of a subject compound. In certain cases, the disease is selected from hyperlipidemia, type 2 diabetes, fatty liver disease, obesity, cardiovascular disease and stroke. In certain cases, the symptom is selected from abdominal obesity, insulin resistance, hyperinsulinemia, high levels of blood fats, increased blood pressure, and elevated serum lipids.

Compounds

As summarized above, aspects of the disclosure include mitochondrial modulator compounds. The subject compounds generally include a head group linked to a charged moiety. In certain cases, the head group is selected from a thiazole, an oxadiazole, a tetrazole, a triazine, and a guanidine. The linker between the head group and the charged moiety can include an ester, a thioester or an amide moiety. In certain cases, the linker is cleavable. In certain cases, the linker is a group modified for modulating the stability of the subject compounds (e.g., to modify the rate of hydrolysis of the subject compound under physiological conditions). The charged group includes, but is not limited to, a phosphonium cation, an ammonium cation, a quaternary ammonium cation, a pyridinium cation, an imidazolium cation, and a guanidine moiety. The subject compounds are optionally further substituted (e.g., as described herein). Exemplary compounds of interest including various head groups linked to various charged groups are set forth in formulae (I)-(IE) and the structures of any of tables 1-8 or compounds A1-A15.

In some cases, the subject compound is of formula (I):

HG-L-X (I)

wherein:

HG is a headgroup selected from a heterocyclic group, a heteroaryl group, and a guanidine, wherein the head group is optionally substituted;

L is a linker; and

X is a charged group,

Provided that the compound is not:

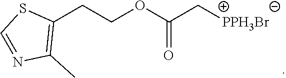

In some embodiments of formula (I), the headgroup (HG) is a heterocyclic group, optionally substituted (e.g., with a substituent as described herein). In certain cases, HG is a heteroaryl group, optionally substituted. In other cases, the head group is a guanidine, optionally substituted.

In certain cases of formula (I), HG is selected from a thiazole, a pyrazole, a thiophene, an oxazole, an oxadiazole, a tetrazole, a triazole, a pyridine, a pyrimidine, a pyrazine, a pyrazine, a triazine, a pyran, an oxazine, a thiazine a morpholine, a thiomorpholine, a piperidine and a piperazine. In certain cases, the headgroup is a thiazole. In certain cases, the head group is a thiazole, optionally substituted (e.g., with a substituent as described herein). In certain cases, the head group is an oxadiazole, optionally substituted. In certain cases, the head group is a tetrazole, optionally substituted. In some cases, the head group is a triazine, optionally substituted. In some cases, the head group is a guanidine, optionally substituted.

In certain embodiments of formula (I), the head group is described by the formula (HG1):

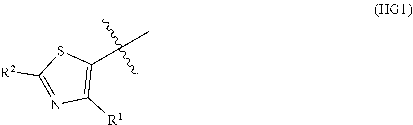

wherein:

R¹ and R² are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG1), R¹ is hydrogen. In certain cases, R¹ is alkyl or substituted alkyl. In certain cases, R¹ is aryl or substituted aryl. In certain cases, R¹ is amino or substituted amino. In certain cases, R¹ is carboxyl or substituted carboxyl. In some cases, R¹ is acyl or substituted acyl. In some cases, R¹ is carboxamide or substituted carboxamide. In certain cases, R¹ is thiol or substituted thiol. In some cases, R¹ is alkoxy or substituted alkoxy. In certain cases, R¹ is halogen.

In certain embodiments of formula (HG1), R² is hydrogen. In certain cases, R² is alkyl or substituted alkyl. In certain cases, R² is aryl or substituted aryl. In certain cases, R² is amino or substituted amino. In certain cases, R² is carboxyl or substituted carboxyl. In some cases, R² is acyl or substituted acyl. In some cases, R² is carboxamide or substituted carboxamide. In certain cases, R² is thiol or substituted thiol. In some cases, R² is alkoxy or substituted alkoxy. In certain cases, R² is halogen.

In certain embodiments of formula (HG1), R¹ is alkyl and R² is hydrogen. In certain cases, R¹ is methyl. Accordingly, in some cases, the compound of formula (HG1) is described by the formula (HG1a):

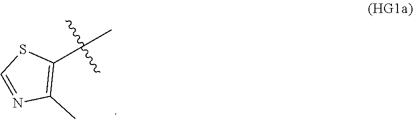 [SMILES: *c1scnc1C]

In certain embodiments of formula (I), the head group is described by the formula (HG1):

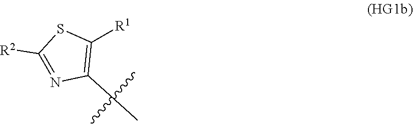

wherein:

R¹ and R² are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG1), R¹ is hydrogen. In certain cases, R¹ is alkyl or substituted alkyl. In certain cases, R¹ is aryl or substituted aryl. In certain cases, R¹ is amino or substituted amino. In certain cases, R¹ is carboxyl or substituted carboxyl. In some cases, R¹ is acyl or substituted acyl. In some cases, R¹ is carboxamide or substituted carboxamide. In certain cases, R¹ is thiol or substituted thiol. In some cases, R¹ is alkoxy or substituted alkoxy. In certain cases, R¹ is halogen.

In certain embodiments of formula (HG1), R² is hydrogen. In certain cases, R² is alkyl or substituted alkyl. In certain cases, R² is aryl or substituted aryl. In certain cases, R² is amino or substituted amino. In certain cases, R² is carboxyl or substituted carboxyl. In some cases, R² is acyl or substituted acyl. In some cases, R² is carboxamide or substituted carboxamide. In certain cases, R² is thiol or substituted thiol. In some cases, R² is alkoxy or substituted alkoxy. In certain cases, R² is halogen.

In certain embodiments of formula (I), the head group is described by the formula (HG2):

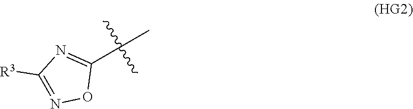

wherein:

R³ is selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG2), R³ is hydrogen. In certain cases, R³ is alkyl or substituted alkyl. In certain cases, R³ is aryl or substituted aryl. In certain cases, R³ is amino or substituted amino. In certain cases, R³ is carboxyl or substituted carboxyl. In some cases, R³ is acyl or substituted acyl. In some cases, R³ is carboxamide or substituted carboxamide. In certain cases, R³ is thiol or substituted thiol. In some cases, R³ is alkoxy or substituted alkoxy. In certain cases, R³ is halogen.

In some cases, the compound of formula (HG2) is described by the formula (HG2a):

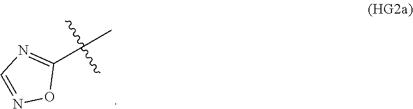 [SMILES: *c1ncno1]

In certain embodiments of formula (I), the head group is described by the formula (HG3):

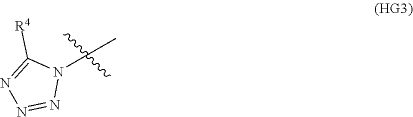

wherein:

R⁴ is selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG3), R⁴ is hydrogen. In certain cases, R⁴ is alkyl or substituted alkyl. In certain cases, R⁴ is aryl or substituted aryl. In certain cases, R⁴ is amino or substituted amino. In certain cases, R⁴ is carboxyl or substituted carboxyl. In some cases, R⁴ is acyl or substituted acyl. In some cases, R⁴ is carboxamide or substituted carboxamide. In certain cases, R⁴ is thiol or substituted thiol. In some cases, R⁴ is alkoxy or substituted alkoxy. In certain cases, R⁴ is halogen.

In some cases, the compound of formula (HG3) is described by the formula (HG3a):

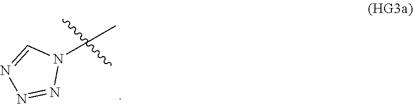 [SMILES: *n1cnnn1]

In certain embodiments of formula (I), the head group is described by the formula (HG4):

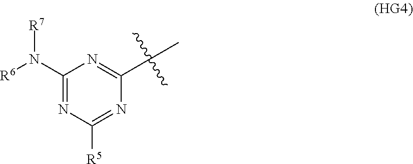

wherein:

R⁵-R⁷ are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG4), R⁵ is hydrogen. In certain cases, R⁵ is alkyl or substituted alkyl. In certain cases, R⁵ is aryl or substituted aryl. In certain cases, R⁵ is amino or substituted amino. In certain cases, R⁵ is carboxyl or substituted carboxyl. In some cases, R⁵ is acyl or substituted acyl. In some cases, R⁵ is carboxamide or substituted carboxamide. In certain cases, R⁵ is thiol or substituted thiol. In some cases, R⁵ is alkoxy or substituted alkoxy. In certain cases, R⁵ is halogen.

In certain embodiments of formula (HG4), R⁶ is hydrogen. In certain cases, R⁶ is alkyl or substituted alkyl. In certain cases, R⁶ is aryl or substituted aryl. In certain cases, R⁶ is amino or substituted amino. In certain cases, R⁶ is carboxyl or substituted carboxyl. In some cases, R⁶ is acyl or substituted acyl. In some cases, R⁶ is carboxamide or substituted carboxamide. In certain cases, R⁶ is thiol or substituted thiol. In some cases, R⁶ is alkoxy or substituted alkoxy. In certain cases, R⁶ is halogen.

In certain embodiments of formula (HG4), R⁷ is hydrogen. In certain cases, R⁷ is alkyl or substituted alkyl. In certain cases, R⁷ is aryl or substituted aryl. In certain cases, R⁷ is amino or substituted amino. In certain cases, R⁷ is carboxyl or substituted carboxyl. In some cases, R⁷ is acyl or substituted acyl. In some cases, R⁷ is carboxamide or substituted carboxamide. In certain cases, R⁷ is thiol or substituted thiol. In some cases, R⁷ is alkoxy or substituted alkoxy. In certain cases, R⁷ is halogen.

In certain embodiments of formula (HG4), R⁵ is hydrogen and R⁵-R⁶ are alkyl. In certain cases, R⁵-R⁶ are methyl. Accordingly, in some cases the formula (HG4) is described by the formula (HG4a):

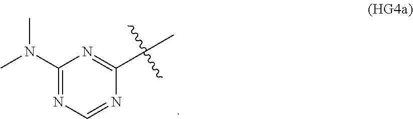 [SMILES: *c1ncnc(N(C)C)n1]

In certain embodiments of formula (I), the head group is described by the formula (HG5):

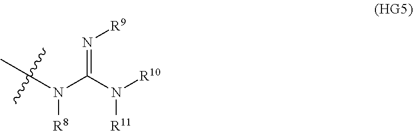

wherein:

R⁸-R¹¹ are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG5), R⁸ is hydrogen. In certain cases, R⁸ is alkyl or substituted alkyl. In certain cases, R⁸ is aryl or substituted aryl. In certain cases, R⁸ is amino or substituted amino. In certain cases, R⁸ is carboxyl or substituted carboxyl. In some cases, R⁸ is acyl or substituted acyl. In some cases, R⁸ is carboxamide or substituted carboxamide. In certain cases, R⁸ is thiol or substituted thiol. In some cases, R⁸ is alkoxy or substituted alkoxy. In certain cases, R⁸ is halogen.

In certain embodiments of formula (HG5), R⁹ is hydrogen. In certain cases, R⁹ is alkyl or substituted alkyl. In certain cases, R⁹ is aryl or substituted aryl. In certain cases, R⁹ is amino or substituted amino. In certain cases, R⁹ is carboxyl or substituted carboxyl. In some cases, R⁹ is acyl or substituted acyl. In some cases, R⁹ is carboxamide or substituted carboxamide. In certain cases, R⁹ is thiol or substituted thiol. In some cases, R⁹ is alkoxy or substituted alkoxy. In certain cases, R⁹ is halogen.

In certain embodiments of formula (HG5), R¹⁰ is hydrogen. In certain cases, R¹⁰ is alkyl or substituted alkyl. In certain cases, R¹⁰ is aryl or substituted aryl. In certain cases, R¹⁰ is amino or substituted amino. In certain cases, R¹⁰ is carboxyl or substituted carboxyl. In some cases, R¹⁰ is acyl or substituted acyl. In some cases, R¹⁰ is carboxamide or substituted carboxamide. In certain cases, R¹⁰ is thiol or substituted thiol. In some cases, R¹⁰ is alkoxy or substituted alkoxy. In certain cases, R¹⁰ is halogen.

In certain embodiments of formula (HG5), R¹¹ is hydrogen. In certain cases, R¹¹ is alkyl or substituted alkyl. In certain cases, R¹¹ is aryl or substituted aryl. In certain cases, R¹¹ is amino or substituted amino. In certain cases, R¹¹ is carboxyl or substituted carboxyl. In some cases, R¹¹ is acyl or substituted acyl. In some cases, R¹¹ is carboxamide or substituted carboxamide. In certain cases, R¹¹ is thiol or substituted thiol. In some cases, R¹¹ is alkoxy or substituted alkoxy. In certain cases, R¹¹ is halogen.

In certain embodiments of formula (HG5), R⁸-R¹¹ are each hydrogen. Accordingly, in some cases the formula (HG5) is described by the formula (HG5a):

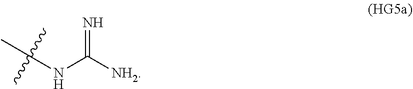 [SMILES: *NC(=N)N]

In certain embodiments of formula (I), the head group is described by the formula (HG6):

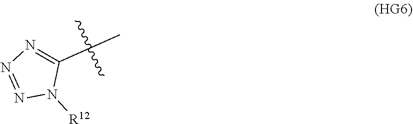

wherein:

R¹² is selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG6), R¹² is hydrogen. In certain cases, R¹² is alkyl or substituted alkyl. In certain cases, R¹² is aryl or substituted aryl. In certain cases, R¹² is amino or substituted amino. In certain cases, R¹² is carboxyl or substituted carboxyl. In some cases, R¹² is acyl or substituted acyl. In some cases, R¹² is carboxamide or substituted carboxamide. In certain cases, R¹² is thiol or substituted thiol. In some cases, R¹² is alkoxy or substituted alkoxy. In certain cases, R¹² is halogen.

In certain cases, the formula (HG6) can be described by formula (HG6a):

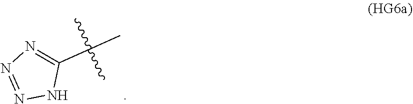 [SMILES: *c1nnn[nH]1]

In certain embodiments of formula (I), the head group is described by the formula (HG7):

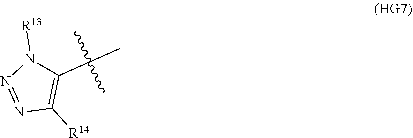

wherein:

R¹³ and R¹⁴ are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG7), R¹³ is hydrogen. In certain cases, R¹³ is alkyl or substituted alkyl. In certain cases, R¹³ is aryl or substituted aryl. In certain cases, R¹³ is amino or substituted amino. In certain cases, R¹³ is carboxyl or substituted carboxyl. In some cases, R¹³ is acyl or substituted acyl. In some cases, R¹³ is carboxamide or substituted carboxamide. In certain cases, R¹³ is thiol or substituted thiol. In some cases, R¹³ is alkoxy or substituted alkoxy. In certain cases, R¹³ is halogen.

In certain embodiments of formula (HG7), R¹⁴ is hydrogen. In certain cases, R¹⁴ is alkyl or substituted alkyl. In certain cases, R¹⁴ is aryl or substituted aryl. In certain cases, R¹⁴ is amino or substituted amino. In certain cases, R¹⁴ is carboxyl or substituted carboxyl. In some cases, R¹⁴ is acyl or substituted acyl. In some cases, R¹⁴ is carboxamide or substituted carboxamide. In certain cases, R¹⁴ is thiol or substituted thiol. In some cases, R¹⁴ is alkoxy or substituted alkoxy. In certain cases, R¹⁴ is halogen.

In certain embodiments of formula (HG7), R¹³ and R¹⁴ are hydrogen. Accordingly, in some cases the formula (HG7) is described by the formula (HG7a):

In certain embodiments of formula (I), the head group is described by the formula (HG8):

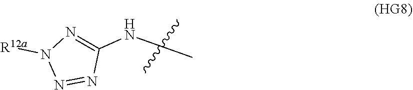

wherein:

R^{12a} is selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG8), R^{12a} is hydrogen. In certain cases, R^{12a} is alkyl or substituted alkyl. In certain cases, R^{12a} is aryl or substituted aryl. In certain cases, R^{12a} is amino or substituted amino. In certain cases, R¹² is carboxyl or substituted carboxyl. In some cases, R^{12a} is acyl or substituted acyl. In some cases, R^{12a} is carboxamide or substituted carboxamide. In certain cases, R^{12a} is thiol or substituted thiol. In some cases, R^{12a} is alkoxy or substituted alkoxy. In certain cases, R^{12a} is halogen.

In certain cases, the formula (HG8) can be described by formula (HG8a):

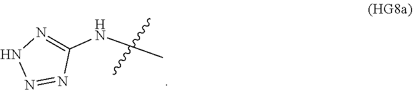 [SMILES: *Nc1nn[nH]n1]

In certain embodiments of formula (I), the head group is described by the formula (HG9):

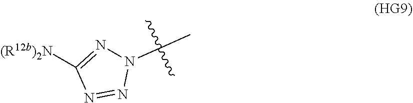

wherein:

each R^{12b} is independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

In certain embodiments of formula (HG9), each R^{12b} is hydrogen. In certain cases, each R^{12b} is alkyl or substituted alkyl.

In certain cases, the formula (HG9) can be described by formula (HG8a):

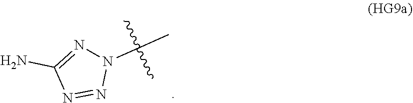 [SMILES: *n1nnc(N)n1]

For any of formulae (HG1)-(HG9a) described herein, [symbol] indicates the bond that is joined to the linking moiety L.

The HG and X moieties of formula (I) are joined together through linking moiety L, where L may be any convenient linking group. The linking groups are chosen to provide for covalent attachment of the HG and X moieties through the linking group, as well as the desired structural relationship of the mitochondrial modulator compound with respect to its intended pharmacokinetic modulating protein. Linking groups of interest may vary widely depending on the nature of the HG and X moieties. The linking group, may be biologically inert. The linking group may be adapted to modulate stability of the subject compound. In certain embodiments, the linker may be designed such that the subject compound is metabolically stable (e.g., remain substantially intact in vivo during the half-life of the compound). In certain embodiments, the linker may be designed such that the subject linker is cleaved in vivo (e.g., via hydrolysis by esterases or peptidases). As used herein, the term “cleavable linker” or “cleavably linked” refers to a linker or a linkage that is selectively breakable using a stimulus (e.g., a physical, chemical or enzymatic stimulus) that leaves the moieties to which the linkages joins intact. Several cleavable linkages have been described in the literature (e.g., Brown (1997) Contemporary Organic Synthesis 4(3); 216-237). And Guillier et al (Chem. Rev. 2000 1000:2091-2157). A disulfide bond (which can be broken by DDT) and a photo-cleavable linker are examples of cleavable linkages.

In certain embodiments, the linker is designed to be cleavably linked in vivo by hydrolysis. In certain cases, the rate of hydrolysis of the subject linker in vivo (e.g., t_{1/2} hydrolysis in vivo) is of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

A variety of linking groups are known to those of skill in the art and find use in the subject compounds. Linkers of interest may include a spacer group terminated at either end with a reactive functionality capable of covalently bonding to the HG or X moieties. Spacer groups of interest include aliphatic and unsaturated hydrocarbon chains, spacers containing heteroatoms such as oxygen (esters, and ethers such as polyethylene glycol) or nitrogen (amides, and polyamines), sulfur (thioesters, and dithioesters), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, esters, amides, thioesters, dithoesters, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the subject bifunctional molecules include heterofunctional compounds, such as azidobenzoyl hydrazide, N-[4-(p-azidosalicylamino)butyl]-3′-[2′-pyridyldithio]propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl 4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like.

Any convenient linker may find use in formula (I), e.g., as described herein. Suitable linkers include, but are not limited to, a moiety comprising a carboxylic acid, an alkyl ester, an aryl ester, a substituted aryl ester, an aldehyde, an amide, an aryl amide, an alkyl halide, a thioester, a dithioester, a sulfonyl ester, an alkyl ketone, an aryl ketone, a substituted aryl ketone, a halosulfonyl, a nitrile, a nitro, a PEG, and a peptide linker.

In certain embodiments of formula (I), the linker is selected from an alkyl ester, an alkyl thioester, an alkyl dithioester, or an alkyl amide. In certain cases, the alkyl ester, alkyl thioester, alkyl dithioester, or alkyl amide is substituted with one or more substituents (e.g., as described herein). In certain instances, the alkyl ester, alkyl thioester or alkyl amide is substituted at the alpha carbon. In certain cases, the linker is an alkyl ester, alkyl thioester or alkyl amide that further includes a PEG moiety.

In certain embodiments of formula (I), L comprises a straight or branched alkyl. In certain cases, L comprises a lower alkyl group, e.g., methyl, ethyl, propyl, butyl, pentyl, or hexyl. In certain cases, L comprises a substituted alkyl group. In certain cases, L comprises a substituted lower alkyl group. In certain cases, L comprises a polyethylene glycol (PEG) or substituted PEG. In certain other cases, L is a peptide. In certain cases, L is a linear linker of 1-12 atoms in length, such as 1-10, 1-8 or 1-6 atoms in length, e.g., 1, 2, 3, 4, 5 or 6 atoms in length. The linker L can be a (C1-12)alkyl linker or a substituted (C1-12)alkyl linker, optionally substituted with a heteroatom or linking functional group, such as an ester (—CO₂—), amido (CONH), carbamate (OCONH), ether (—O—), thioether (—S—), thioester (—C(S)O—, or —C(O)S), dithioester (—CS₂—) and/or amino group (—NR— where R is H or alkyl). In certain cases, the linker L can include a keto (C═O) group. In certain cases, the keto group together with an amino, thiol or ether group in the linker chain can provide an amido, an ester or thioester group linkage. In certain cases, the linker L can include a thiocarbonyl (C═S) group. In certain cases, the thiocarbonyl group together with an amino, thiol or ether group in the linker chain can provide a thioamide, or a thioester group linkage.

In certain embodiments of a compound of formula (I), the linker is described by the formula (L1):

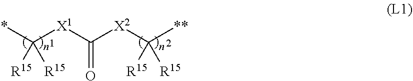

wherein:

* represents the point of connection to HG;

** represents the point of connection to X;

X¹ and X² are each independently selected from C(R¹⁵)₂, C(R¹⁵)₂(OCH₂CH₂O)ₙ₃, O, S and NR¹⁶;

each R¹⁵ is independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino and hydroxyl;

n¹ an integer from 0 to 10;

n² is an integer from 0 to 10; and

n³ is an integer from 1 to 20.

In some embodiments of formula (L1), X¹ is oxygen. In some cases, X¹ is sulfur. In some cases, X¹ is NR¹⁶, wherein R¹⁶ is selected from H, alkyl, and substituted alkyl. In certain cases, R¹⁶ is hydrogen. In some cases, R¹⁶ is alkyl or substituted alkyl. In certain embodiments X¹ is C(R¹⁵)₂, wherein R¹⁵ is selected from H, alkyl, and substituted alkyl. In certain cases, each R¹⁵ is hydrogen. In certain cases, at least one R¹⁵ group is a substituent other than hydrogen.

In some embodiments of formula (L1), X² is oxygen. In some cases, X² is sulfur. In some cases, X² is NR¹⁶, wherein R¹⁶ is selected from H, alkyl, and substituted alkyl. In certain cases, R¹⁶ is hydrogen. In some cases, R¹⁶ is alkyl or substituted alkyl. In certain embodiments X² is C(R¹⁵)₂, wherein each R¹⁵ is independently selected from H, alkyl, and substituted alkyl. In certain cases, each R¹⁵ is hydrogen. In certain cases, at least one R¹⁵ group is a substituent other than hydrogen.

In some embodiments of formula (L1), X¹ is oxygen, and X² is CH₂. In some cases, X¹ is sulfur, and X² is CH₂. In some cases, X¹ is NR¹⁶, wherein R¹⁶ is selected from H, alkyl, and substituted alkyl, and X² is CH₂. In certain cases, R¹⁶ is hydrogen. In some cases, R¹⁶ is alkyl or substituted alkyl.

In some embodiments of formula (L1), X² is oxygen, and X¹ is CH₂. In some cases, X² is sulfur, and X¹ is CH₂. In some cases, X² is NR¹⁶, wherein R¹⁶ is selected from H, alkyl, and substituted alkyl, and X¹ is CH₂. In certain cases, R¹⁶ is hydrogen. In some cases, R¹⁶ is alkyl or substituted alkyl.

In certain embodiments of formula (L1), the linker is described by a structure selected from any one of (L2)-(L5):

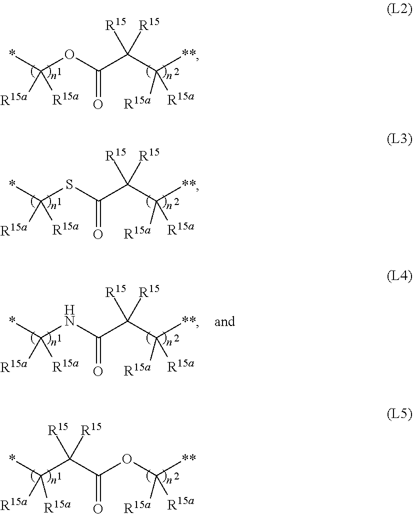

wherein:

* represents the point of connection to HG;

** represents the point of connection to X;

R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

n¹ an integer from 0 to 10; and

n² is an integer from 0 to 10.

In certain embodiments of any one of formulae (L1)-(L5), n¹ is 0. In certain cases, n² is 0. In certain cases, n¹ and n² are independently selected from an integer from 0 to 10, such as 0-8, 0-6, or 0 to 2. In some cases, the sum of n¹ and n² is less than 10, such as less than 8, less than 6, or even less. In some cases, n¹ is an integer from 1-6, and n² is 0. In some cases, n¹ is an integer from 1-6, and n² is 1. In some cases, n² is an integer from 1-6, and n¹ is 0. In some cases, n² is an integer from 1-6, and n¹ is 1.

In certain cases of any one of formulae (L2)-(L5), each R¹⁵ and R^{15a} group is hydrogen. In certain cases of any one of formulae (L2)-(L5), each R^{15a} group is hydrogen and at least one R¹⁵ group is a substituent other than hydrogen. In some cases of any one of formulae (L2)-(L5), both R¹⁵ groups are substituents other than hydrogen.

In certain embodiments of any one of formulae (L1)-(L5), the carbonyl group (i.e., C═O) is a thiocarbonyl group (i.e., C═S).

In certain embodiments, of formula (L1), the linker is described by a structure selected from any one of (B1)-(B11):

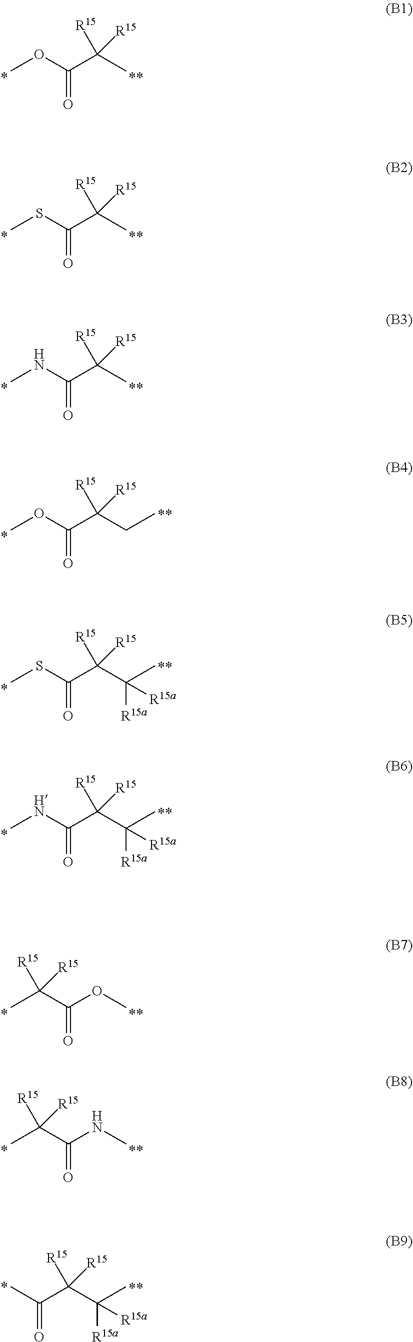

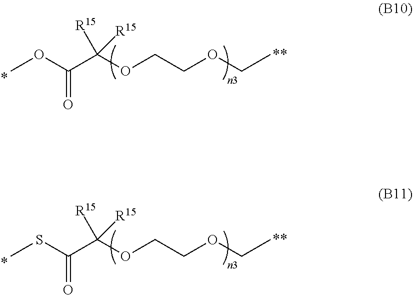

wherein:

* represents the point of connection to HG;

** represents the point of connection to X;

R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

n¹ an integer from 0 to 10;

n² is an integer from 0 to 10; and

n³ is an integer from 1 to 20.

In certain cases of any one of formulae (B1)-(B11), each R¹⁵ and R^{15a} group is hydrogen. In certain cases of any one of formulae (B1-(B11), at least one R¹⁵ group is a substituent other than hydrogen. In some cases of any one of formulae (B1)-(B11), both R¹⁵ groups are substituents other than hydrogen.

In certain embodiments of any one of formulae (B1)-(B11), the carbonyl group (i.e., C═O) is a thiocarbonyl group (i.e., C═S).

In certain embodiments, the formula (I) is described by the formula (IA) or (IB):

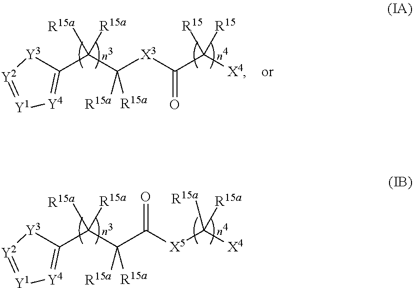

wherein:

Y¹, Y² and Y⁴ are each independently selected from N and CR¹⁵; Y³ is selected from S, O, NR¹⁶, and C(R¹⁵)₂;

X³ and X⁵ are each independently selected from C(R¹⁵)₂, O, S and NR¹⁶;

each R¹⁵ and R^{15a} are independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;
each R¹⁶ is independently selected from hydrogen, alkyl, substituted alkyl, aryl, substituted aryl, carboxyl, acyl, substituted acyl, amino, substituted amino, and hydroxyl;

X⁴ is a charged group;

n³ an integer from 0 to 10; and

n⁴ is an integer from 1 to 10.

In certain embodiments of formula (IA) or (IB), Y¹ is N, Y² is CR¹⁵, Y³ is S and Y⁴ is CR¹⁵, wherein each R¹⁵ is independently selected from hydrogen, alkyl, and substituted alkyl. In certain cases, Y¹ is CR¹⁵, Y² is N, Y³ is 0 and Y⁴ is N, wherein R¹⁵ is selected from hydrogen, alkyl, and substituted alkyl. In certain cases, Y¹ is N, Y² is N, Y³ is NR¹⁶ and Y⁴ is N, wherein R¹⁶ is selected from hydrogen, and amino. In certain cases, Y′ is N, Y² is N, Y³ is NR¹⁶ and Y⁴ is CR¹⁵, wherein R¹⁶ is selected from hydrogen and amino.

In some embodiments of formula (IA) or (IB), X³ is oxygen. In some cases, X³ is sulfur. In some cases, X³ is NR¹⁶, wherein R¹⁶ is selected from H, alkyl, and substituted alkyl. In certain cases, R¹⁶ is hydrogen. In some cases, R¹⁶ is alkyl or substituted alkyl. In certain embodiments X³ is C(R¹⁵), wherein each R¹⁵ is selected from H, alkyl, and substituted alkyl. In certain cases, each R¹⁵ is hydrogen. In certain cases, at least one R¹⁵ group is a substituent other than hydrogen.

In some embodiments of formula (IA) or (IB), X⁵ is oxygen. In some cases, X⁵ is sulfur. In some cases, X⁵ is NR¹⁶, wherein R¹⁶ is selected from H, alkyl, and substituted alkyl. In certain cases, R¹⁶ is hydrogen. In some cases, R¹⁶ is alkyl or substituted alkyl. In certain embodiments X⁵ is C(R¹⁵)₂, wherein each R¹⁵ is independently selected from H, alkyl, and substituted alkyl. In certain cases, each R¹⁵ is hydrogen. In certain cases, at least one R¹⁵ group is a substituent other than hydrogen.

In certain embodiments of any one of formula (IA) or (IB), n³ is 0. In certain cases, n³ is 1. In certain cases, n⁴ is 1. In certain cases, n³ and n⁴ are independently selected from an integer from 1 to 10, such as 1-8, 1-6, or 1 to 2. In some cases, the sum of n³ and n⁴ is less than 10, such as less than 8, less than 6, or even less. In some cases, n³ is an integer from 1-6, and n⁴ is 1. In some cases, n³ is an integer from 1-3, and n⁴ is 1. In some cases, n⁴ is an integer from 1-6, and n³ is 0. In some cases, n⁴ is an integer from 1-6, and n³ is 1. In some cases, n⁴ is an integer from 1-3, and n³ is 1.

In certain cases of any one of formulae (IA)-(IB), each R¹⁵ and R^{15a} group is hydrogen. In certain cases of any one of formulae (IA)-(IB), each R^{15a} group is hydrogen and at least one R¹⁵ group is a substituent other than hydrogen. In some cases of any one of formulae (IA)-(IB), both R¹⁵ groups are substituents other than hydrogen.

In certain embodiments, the compound is described by the formula (IC) or (ID):

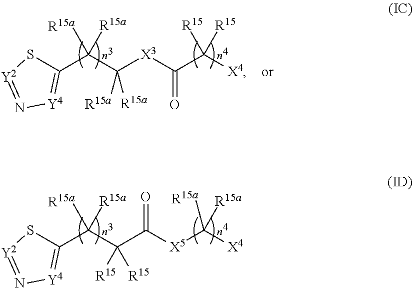

wherein:

Y² and Y⁴ are each CR¹⁵;

X³ and X⁵ are each independently selected from C(R¹⁵)₂, O, S and NR¹⁶;

each R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino, and hydroxyl;

X⁴ is a charged group;

n³ an integer from 0 to 10; and

n⁴ is an integer from 1 to 10.

In some embodiments of formula (IC) or (ID), X³ is oxygen. In some cases, X³ is sulfur. In some cases, X³ is NR¹⁶, wherein R¹⁶ is selected from H, alkyl, and substituted alkyl. In certain cases, R¹⁶ is hydrogen. In some cases, R¹⁶ is alkyl or substituted alkyl. In certain embodiments X³ is C(R¹⁵), wherein each R¹⁵ is independently selected from H, alkyl, and substituted alkyl. In certain cases, each R¹⁵ is hydrogen. In certain cases, at least one R¹⁵ group is a substituent other than hydrogen.

In some embodiments of formula (IC) or (ID), X⁵ is oxygen. In some cases, X⁵ is sulfur. In some cases, X⁵ is NR¹⁶, wherein R¹⁶ is selected from H, alkyl, and substituted alkyl. In certain cases, R¹⁶ is hydrogen. In some cases, R¹⁶ is alkyl or substituted alkyl. In certain embodiments X⁵ is C(R¹⁵)₂, wherein each R¹⁵ is independently selected from H, alkyl, and substituted alkyl. In certain cases, each R¹⁵ is hydrogen. In certain cases, at least one R¹⁵ group is a substituent other than hydrogen.

In certain embodiments of any one of formula (IC) or (ID), n³ is 0. In certain cases, n³ is 1. In certain cases, n⁴ is 1. In certain cases, n³ and n⁴ are independently selected from an integer from 1 to 10, such as 1-8, 1-6, or 1 to 2. In some cases, the sum of n³ and n⁴ is less than 10, such as less than 8, less than 6, or even less. In some cases, n³ is an integer from 1-6, and n⁴ is 1. In some cases, n³ is an integer from 1-3, and n⁴ is 1. In some cases, n⁴ is an integer from 1-6, and n³ is 0. In some cases, n⁴ is an integer from 1-6, and n³ is 1. In some cases, n⁴ is an integer from 1-3, and n³ is 1.

In certain embodiments of formula (IC) or (ID), Y⁴ is CH. In certain cases, Y⁴ is CR¹⁵ and R¹⁵ is alkyl or substituted alkyl. In certain cases, Y⁴ is CR¹⁵ and R¹⁵ is aryl or substituted aryl. In certain cases, Y⁴ is CR¹⁵ and R¹⁵ is amino or substituted amino. In certain cases, Y⁴ is CR¹⁵ and R¹⁵ is carboxyl or substituted carboxyl. In some cases, Y⁴ is CR¹⁵ and R¹⁵ is acyl or substituted acyl. In some cases, Y⁴ is CR¹⁵ and R¹⁵ is carboxamide or substituted carboxamide. In certain cases, Y⁴ is CR¹⁵ and R¹⁵ is thiol or substituted thiol. In some cases, Y⁴ is CR¹⁵ and R¹⁵ is alkoxy or substituted alkoxy. In certain cases, Y⁴ is CR¹⁵ and R¹⁵ is halogen.

In certain embodiments of formula (IC) or (ID), Y² is CR¹⁵ and R¹⁵ is hydrogen. In certain cases, Y² is CR¹⁵ and R¹⁵ is alkyl or substituted alkyl. In certain cases, Y² is CR¹⁵ and R¹⁵ is aryl or substituted aryl. In certain cases, Y² is CR¹⁵ and R¹⁵ is amino or substituted amino. In certain cases, Y² is CR¹⁵ and R¹⁵ is carboxyl or substituted carboxyl. In some cases, Y² is CR¹⁵ and R¹⁵ is acyl or substituted acyl. In some cases, Y² is CR¹⁵ and R¹⁵ is carboxamide or substituted carboxamide. In certain cases, Y² is CR¹⁵ and R¹⁵ is thiol or substituted thiol. In some cases, Y² is CR¹⁵ and R¹⁵ is alkoxy or substituted alkoxy. In certain cases, Y² is CR¹⁵ and R¹⁵ is halogen.

In certain embodiments of formula (IC) or (ID), Y⁴ is CR¹⁵ and R¹⁵ is alkyl; and Y² is CR¹⁵ and R¹⁵ is hydrogen.

In certain embodiments, the compound is described by the compound (IE):

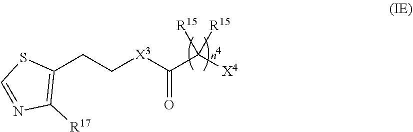

wherein:

X³ is selected from C(R¹⁵)₂, O, S and NR¹⁶;

each R¹⁵, and R¹⁷ are independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino, and hydroxyl;

X⁴ is a charged group; and

n⁴ is an integer from 1 to 10.

In some embodiments of formula (IE), X³ is oxygen. In some cases, X³ is sulfur. In some cases, X³ is NR¹⁶, wherein R¹⁶ is selected from H, alkyl, and substituted alkyl. In certain cases, R¹⁶ is hydrogen. In some cases, R¹⁶ is alkyl or substituted alkyl. In certain embodiments X³ is C(R¹⁵)₂, wherein each R¹⁵ is independently selected from H, alkyl, and substituted alkyl. In certain cases, each R¹⁵ is hydrogen. In certain cases, at least one R¹⁵ group is a substituent other than hydrogen.

In certain embodiments of any one of formula (IE), n⁴ is 1. In certain cases, n⁴ is selected from an integer from 1 to 10, such as 1-8, 1-6, or 1 to 2. In some cases, n⁴ is less than 10, such as less than 8, less than 6, or even less. In some cases, n⁴ is an integer from 1-6. In some cases, n⁴ is an integer from 1-3.

In certain embodiments of formula (IE), R¹⁷ is hydrogen. In certain cases, R¹⁷ is alkyl or substituted alkyl. In certain cases, R¹⁷ is aryl or substituted aryl. In certain cases, R¹⁷ is amino or substituted amino. In certain cases, R¹⁷ is carboxyl or substituted carboxyl. In some cases, R¹⁷ is acyl or substituted acyl. In some cases, R¹⁷ is carboxamide or substituted carboxamide. In certain cases, R¹⁷ is thiol or substituted thiol. In some cases, R¹⁷ is alkoxy or substituted alkoxy. In certain cases, R¹⁷ is halogen.

In certain cases of any of formulae (I)-(IE), the charged group is selected from a phosphonium cation, an ammonium cation, a quaternary ammonium cation, a pyridinium cation, an imidazolium cation and a guanidine moiety.

In certain embodiments of any of formulae (I)-(IE), the charged group is a triphenylphosphonium cation. In certain cases, the charged group is represented by the formula (X1):

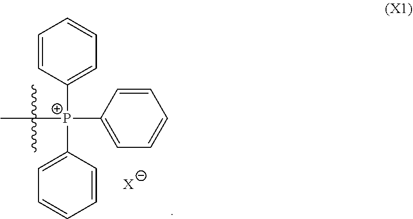

In certain embodiments of formula (X1), the counterion is a halide. In certain cases, the counter ion (X⁻) is bromide.

In certain embodiments of any one of any of formula (I)-(IE), the charged group is a triethylammonium ion. In certain cases, the charged group is represented by the formula (X2):

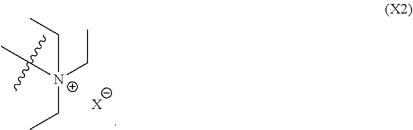

In certain embodiments of formula (X2), the counterion is a halide. In certain cases, the counter ion (X⁻) is bromide.

In certain embodiments of any one of any of formula (I)-(IE), the charged group is an ammonium ion represented by the formula (X3):

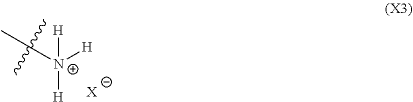

In certain embodiments of formula (X3), the counterion is a halide. In certain cases, the counter ion (X⁻) is bromide.

In certain embodiments of any of formulae (I)-(IE), the charged group is a a pyridinium cation. In certain cases, the charged group is represented by the formula (X4) or (X6):

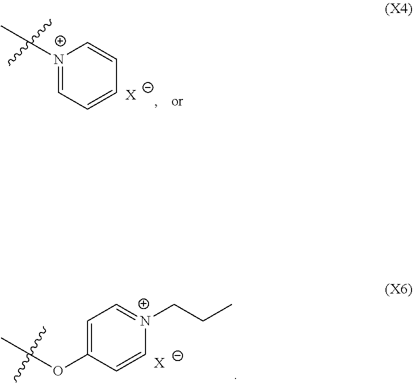

In certain embodiments of formula (X4) or (X6), the counterion is a halide. In certain cases, the counter ion (X⁻) is bromide.

In certain embodiments of any of formulae (I)-(IE), the charged group is an imidazolium cation. In certain cases, the charged group is represented by the formula (X5):

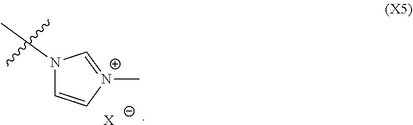

In certain embodiments of formula (X5), the counterion is a halide. In certain cases, the counter ion (X⁻) is bromide.

In certain embodiments of any one of any of formula (I)-(IE), the charged group is a guanidinium cation represented by the formula (X7):

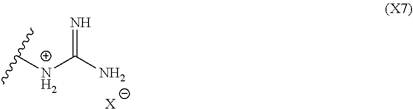

In certain embodiments of formula (X7), the counterion is a halide. In certain cases, the counter ion (X⁻) is bromide.

In certain embodiments of any one of any of formula (I)-(IE), the charged group is a arginine cation represented by the formula (X8) or (X9):

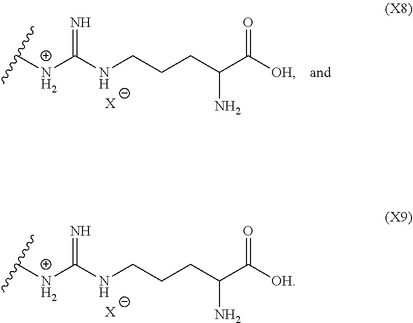

In certain embodiments of formula (X8) or (X9), the counterion is a halide. In certain cases, the counter ion (X⁻) is bromide.

In certain embodiments the compound is described by a structure in any one of Table 1 to Table 8:

TABLE 1
Compounds including the headgroup HG1a.

| Compound | Structure |
|---|---|
| HG1a-1 | 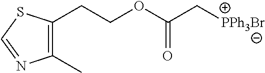 |
| HG1a-2 | 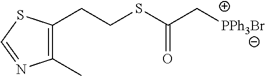 |
| HG1a-3 | 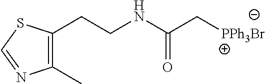 |
| HG1a-4 | 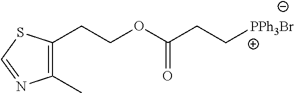 |
| HG1a-5 | 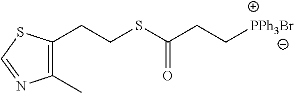 |
| HG1a-6 | 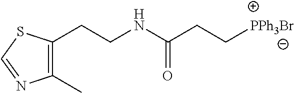 |
| HG1a-7 | 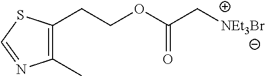 |
| HG1a-8 | 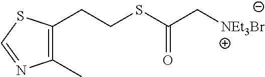 |
| HG1a-9 | 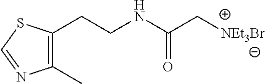 |
| HG1a-10 | 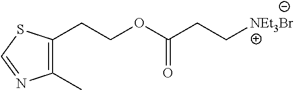 |
| HG1a-11 | 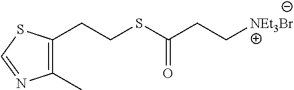 |
| HG1a-12 | 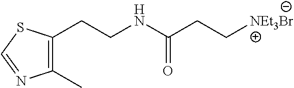 |
| HG1a-13 | 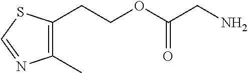 |
| HG1a-14 | 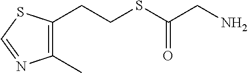 |
| HG1a-15 | 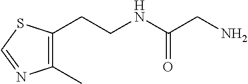 |
| HG1a-16 | 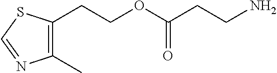 |
| HG1a-17 | 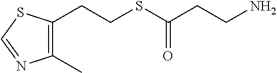 |
| HG1a-18 | 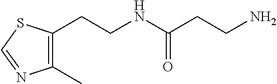 |
| HG1a-19 | 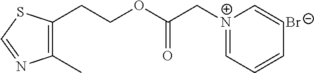 [SMILES: Cc1ncsc1CCOC(=O)C[n+]1ccccc1 ; [Br-]] |
| HG1a-20 | 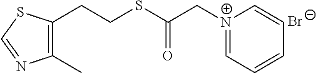 [SMILES: Cc1ncsc1CCSC(=O)C[n+]1ccccc1 ; [Br-]] |
| HG1a-21 | 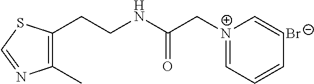 [SMILES: Cc1ncsc1CCNC(=O)C[n+]1ccccc1 ; [Br-]] |
| HG1a-22 | 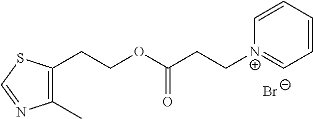 [SMILES: Cc1ncsc1CCOC(=O)CC[n+]1ccccc1 ; [Br-]] |
| HG1a-23 | 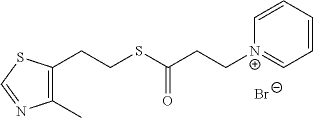 [SMILES: Cc1ncsc1CCSC(=O)CC[n+]1ccccc1 ; [Br-]] |
| HG1a-24 | 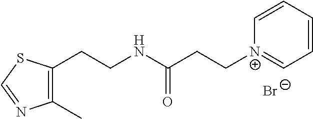 [SMILES: Cc1ncsc1CCNC(=O)CC[n+]1ccccc1 ; [Br-]] |
| HG1a-25 | 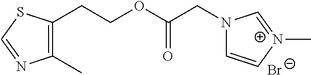 [SMILES: Cc1ncsc1CCOC(=O)Cn1cc[n+](C)c1 ; [Br-]] |
| HG1a-26 | 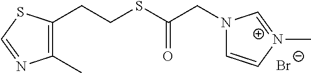 [SMILES: Cc1ncsc1CCSC(=O)Cn1cc[n+](C)c1 ; [Br-]] |
| HG1a-27 | 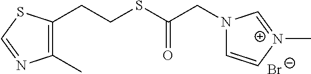 [SMILES: Cc1ncsc1CCSC(=O)Cn1cc[n+](C)c1 ; [Br-]] |
| HG1a-28 | 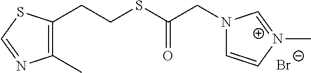 [SMILES: Cc1ncsc1CCSC(=O)Cn1cc[n+](C)c1 ; [Br-]] |
| HG1a-29 | 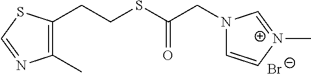 [SMILES: Cc1ncsc1CCSC(=O)Cn1cc[n+](C)c1 ; [Br-]] |
| HG1a-30 | 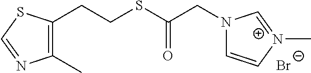 [SMILES: Cc1ncsc1CCSC(=O)Cn1cc[n+](C)c1 ; [Br-]] |
| HG1a-31 | 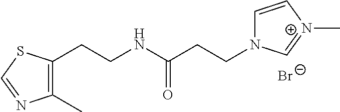 [SMILES: Cc1ncsc1CCNC(=O)CCn1cc[n+](C)c1 ; [Br-]] |
| HG1a-32 | 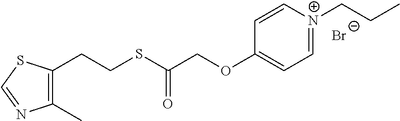 [SMILES: CCC[n+]1ccc(OCC(=O)SCCc2scnc2C)cc1 ; [Br-]] |
| HG1a-33 | 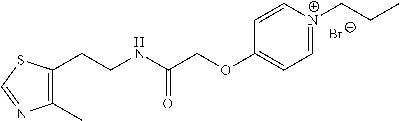 [SMILES: CCC[n+]1ccc(OCC(=O)NCCc2scnc2C)cc1 ; [Br-]] |
| HG1a-34 | 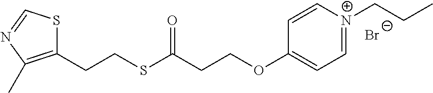 [SMILES: CCC[n+]1ccc(OCCC(=O)SCCc2scnc2C)cc1 ; [Br-]] |
| HG1a-35 | 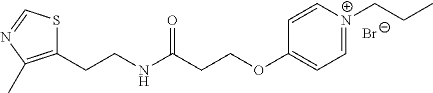 [SMILES: CCC[n+]1ccc(OCCC(=O)NCCc2scnc2C)cc1 ; [Br-]] |
| HG1a-36 | 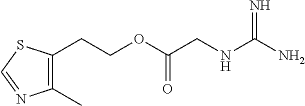 [SMILES: Cc1ncsc1CCOC(=O)CNC(=N)N] |
| HG1a-37 | 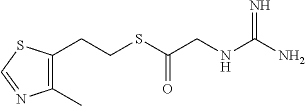 [SMILES: Cc1ncsc1CCSC(=O)CNC(=N)N] |
| HG1a-38 | 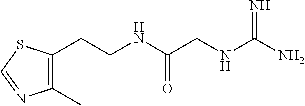 [SMILES: Cc1ncsc1CCNC(=O)CNC(=N)N] |
| HG1a-39 | 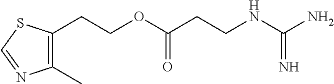 [SMILES: Cc1ncsc1CCOC(=O)CCNC(=N)N] |
| HG1a-40 | 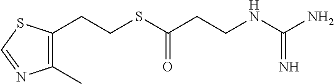 [SMILES: Cc1ncsc1CCSC(=O)CCNC(=N)N] |
| HG1a-41 | 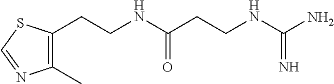 [SMILES: Cc1ncsc1CCNC(=O)CCNC(=N)N] |
| HG1a-42 | 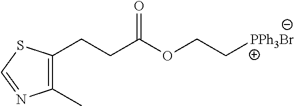 |
| HG1a-43 | 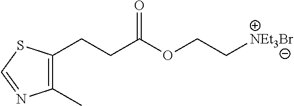 |
| HG1a-44 | 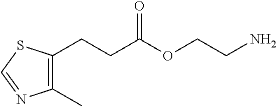 |
| HG1a-45 | 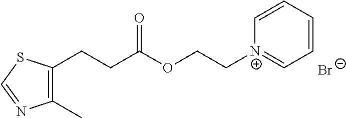 [SMILES: Cc1ncsc1CCC(=O)OCC[n+]1ccccc1 ; [Br-]] |
| HG1a-46 | 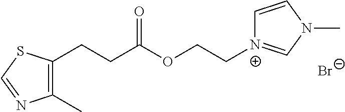 [SMILES: Cc1ncsc1CCC(=O)OCC[n+]1ccn(C)c1 ; [Br-]] |
| HG1a-47 | 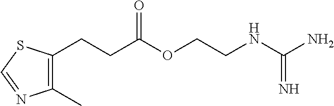 [SMILES: Cc1ncsc1CCC(=O)OCCNC(=N)N] |
| HG1a-48 | 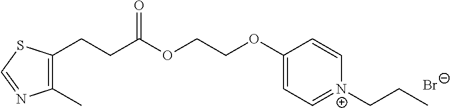 [SMILES: CCC[n+]1ccc(OCCOC(=O)CCc2scnc2C)cc1 ; [Br-]] |
| HG1a-49 | 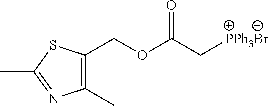 |
| HG1a-50 | 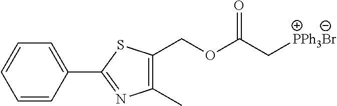 |
| HG1a-51 | 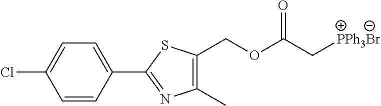 |
| HG1a-52 | 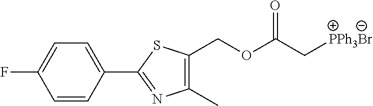 |
| HG1a-53 | 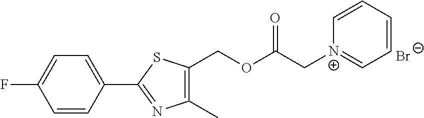 [SMILES: Cc1nc(-c2ccc(F)cc2)sc1COC(=O)C[n+]1ccccc1 ; [Br-]] |
| HG1a-54 | 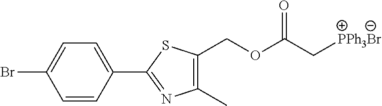 |
| HG1a-55 | 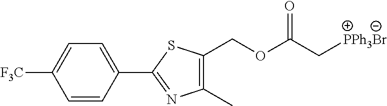 |
| HG1a-56 | 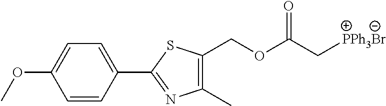 |
| HG1a-57 | 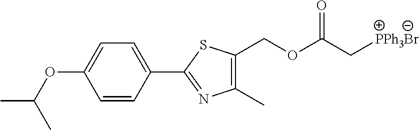 |
| HG1a-58 | 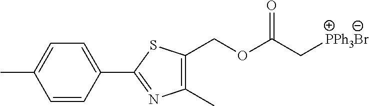 |
| HG1a-59 | 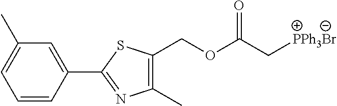 |
| HG1a-60 | 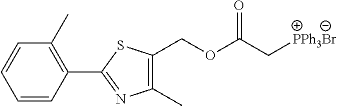 |
| HG1a-61 | 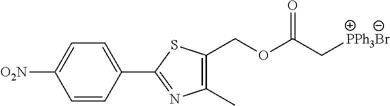 |
| HG1a-62 | 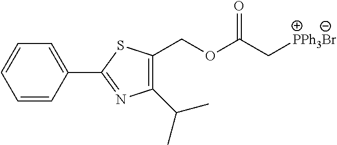 |
| HG1a-63 | 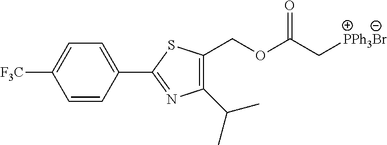 |
| HG1a-64 | 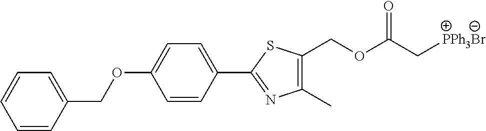 |
| HG1a-65 | 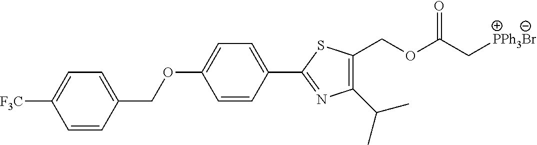 |
| HG1a-66 | 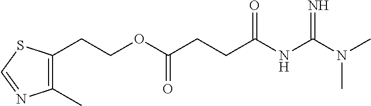 [SMILES: Cc1ncsc1CCOC(=O)CCC(=O)NC(=N)N(C)C] |

TABLE 2
Compounds including the headgroup HG1b.

| Compound | Structure |
|---|---|
| HG1b-1 | 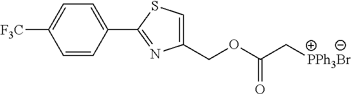 |
| HG1b-2 | 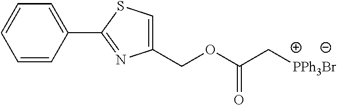 |
| HG1b-3 | 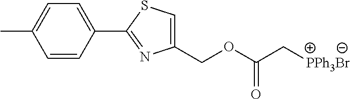 |
| HG1b-4 | 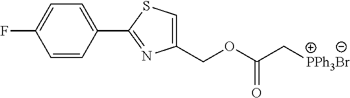 |
| HG1b-5 | 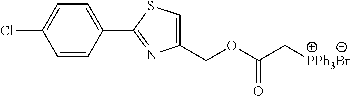 |
| HG1b-6 | 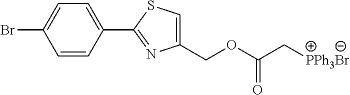 |

TABLE 3
Compounds including the headgroup HG2a.

| Compound | Structure |
|---|---|
| HG2a-1 | 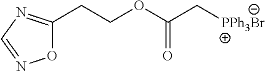 |
| HG2a-2 | 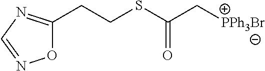 |
| HG2a-3 | 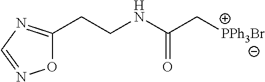 |
| HG2a-4 | 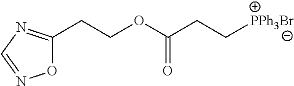 |
| HG2a-5 | 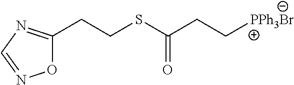 |
| HG2a-6 | 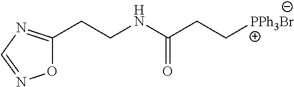 |
| HG2a-7 | 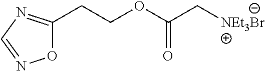 |
| HG2a-8 | 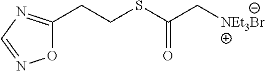 |
| HG2a-9 | 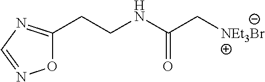 |
| HG2a-10 | 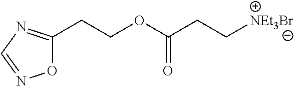 |
| HG2a-11 | 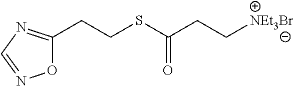 |
| HG2a-12 | 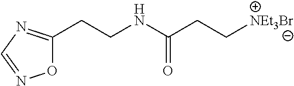 |
| HG2a-13 | 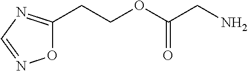 [SMILES: NCC(=O)OCCc1ncno1] |
| HG2a-14 | 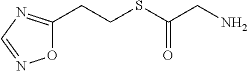 [SMILES: NCC(=O)SCCc1ncno1] |
| HG2a-15 | 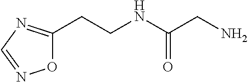 [SMILES: NCC(=O)NCCc1ncno1] |
| HG2a-16 | 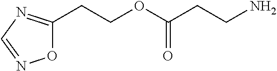 [SMILES: NCCC(=O)OCCc1ncno1] |
| HG2a-17 | 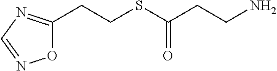 [SMILES: NCCC(=O)SCCc1ncno1] |
| HG2a-18 | 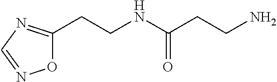 [SMILES: NCCC(=O)NCCc1ncno1] |
| HG2a-19 | 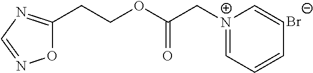 [SMILES: O=C(C[n+]1ccccc1)OCCc1ncno1 ; [Br-]] |
| HG2a-20 | 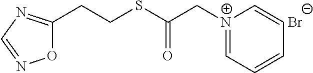 [SMILES: O=C(C[n+]1ccccc1)SCCc1ncno1 ; [Br-]] |
| HG2a-21 | 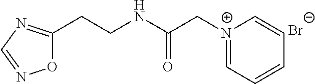 [SMILES: O=C(C[n+]1ccccc1)NCCc1ncno1 ; [Br-]] |
| HG2a-22 | 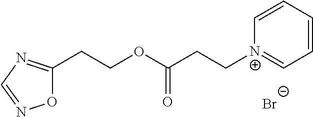 [SMILES: O=C(CC[n+]1ccccc1)OCCc1ncno1 ; [Br-]] |
| HG2a-23 | 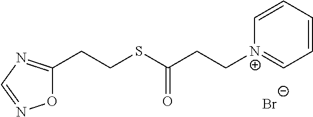 [SMILES: O=C(CC[n+]1ccccc1)SCCc1ncno1 ; [Br-]] |
| HG2a-24 | 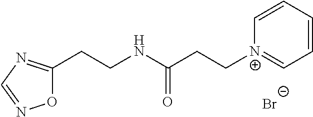 [SMILES: O=C(CC[n+]1ccccc1)NCCc1ncno1 ; [Br-]] |
| HG2a-25 | 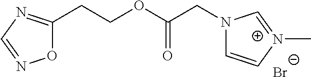 [SMILES: C[n+]1ccn(CC(=O)OCCc2ncno2)c1 ; [Br-]] |
| HG2a-26 | 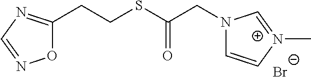 [SMILES: C[n+]1ccn(CC(=O)SCCc2ncno2)c1 ; [Br-]] |
| HG2a-27 | 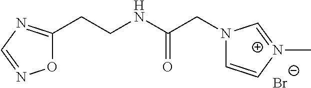 [SMILES: C[n+]1ccn(CC(=O)NCCc2ncno2)c1 ; [Br-]] |
| HG2a-28 | 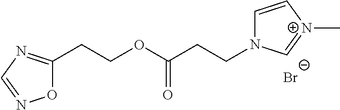 [SMILES: C[n+]1ccn(CCC(=O)OCCc2ncno2)c1 ; [Br-]] |
| HG2a-29 | 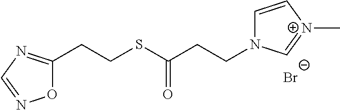 [SMILES: C[n+]1ccn(CCC(=O)SCCc2ncno2)c1 ; [Br-]] |
| HG2a-30 | 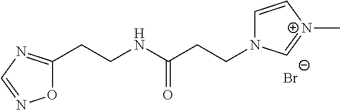 [SMILES: C[n+]1ccn(CCC(=O)NCCc2ncno2)c1 ; [Br-]] |
| HG2a-31 | 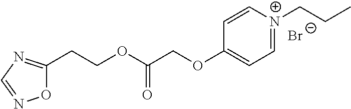 [SMILES: CCC[n+]1ccc(OCC(=O)OCCc2ncno2)cc1 ; [Br-]] |
| HG2a-32 | 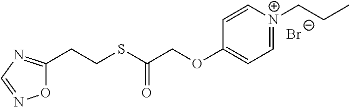 [SMILES: CCC[n+]1ccc(OCC(=O)SCCc2ncno2)cc1 ; [Br-]] |
| HG2a-33 | 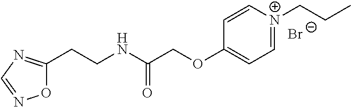 [SMILES: CCC[n+]1ccc(OCC(=O)NCCc2ncno2)cc1 ; [Br-]] |
| HG2a-34 | 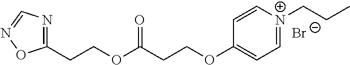 [SMILES: CCC[n+]1ccc(OCCC(=O)OCCc2ncno2)cc1 ; [Br-]] |
| HG2a-35 | 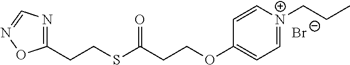 [SMILES: CCC[n+]1ccc(OCCC(=O)SCCc2ncno2)cc1 ; [Br-]] |
| HG2a-36 | 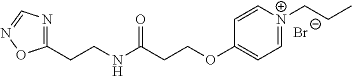 [SMILES: CCC[n+]1ccc(OCCC(=O)NCCc2ncno2)cc1 ; [Br-]] |
| HG2a-37 | 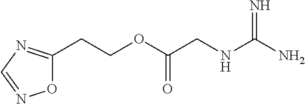 [SMILES: N=C(N)NCC(=O)OCCc1ncno1] |
| HG2a-38 | 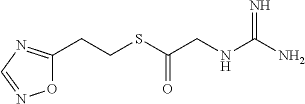 [SMILES: N=C(N)NCC(=O)SCCc1ncno1] |
| HG2a-39 | 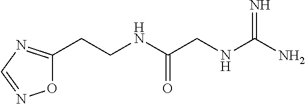 [SMILES: N=C(N)NCC(=O)NCCc1ncno1] |
| HG2a-40 | 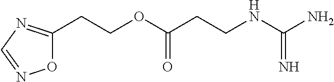 [SMILES: N=C(N)NCCC(=O)OCCc1ncno1] |
| HG2a-41 | 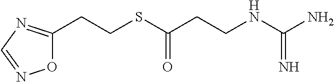 [SMILES: N=C(N)NCCC(=O)SCCc1ncno1] |
| HG2a-42 | 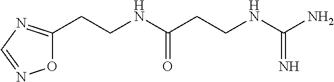 [SMILES: N=C(N)NCCC(=O)NCCc1ncno1] |

TABLE 4
Compounds including the headgroup HG3a.

| Compound | Structure |
|---|---|
| HG3a-1 | 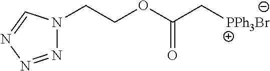 |
| HG3a-2 | 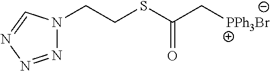 |
| HG3a-3 | 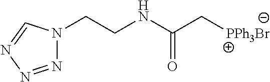 |
| HG3a-4 | 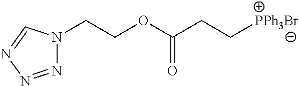 |
| HG3a-5 | 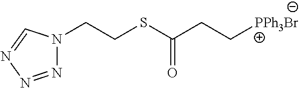 |
| HG3a-6 | 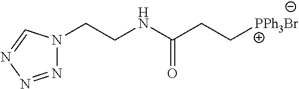 |
| HG3a-7 | 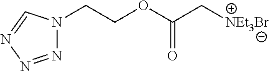 |
| HG3a-8 | 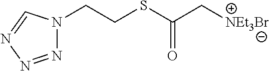 |
| HG3a-9 | 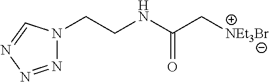 |
| HG3a-10 | 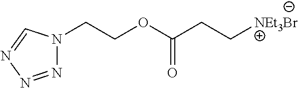 |
| HG3a-11 | 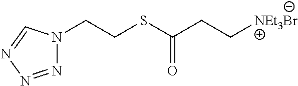 |
| HG3a-12 | 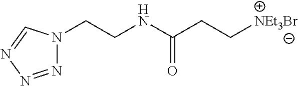 |
| HG3a-13 | 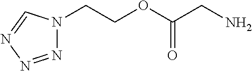 [SMILES: NCC(=O)OCCn1cnnn1] |
| HG3a-14 | 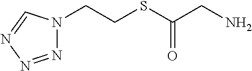 [SMILES: NCC(=O)SCCn1cnnn1] |
| HG3a-15 | 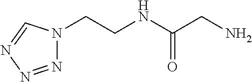 [SMILES: NCC(=O)NCCn1cnnn1] |
| HG3a-16 | 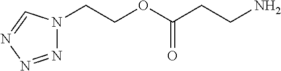 [SMILES: NCCC(=O)OCCn1cnnn1] |
| HG3a-17 | 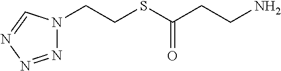 [SMILES: NCCC(=O)SCCn1cnnn1] |
| HG3a-18 | 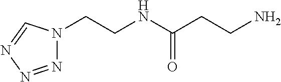 [SMILES: NCCC(=O)NCCn1cnnn1] |
| HG3a-19 | 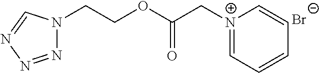 [SMILES: O=C(C[n+]1ccccc1)OCCn1cnnn1 ; [Br-]] |
| HG3a-20 | 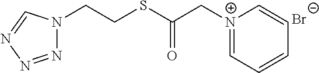 [SMILES: O=C(C[n+]1ccccc1)SCCn1cnnn1 ; [Br-]] |
| HG3a-21 | 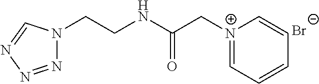 [SMILES: O=C(C[n+]1ccccc1)NCCn1cnnn1 ; [Br-]] |
| HG3a-22 | 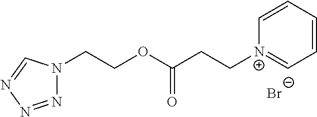 [SMILES: O=C(CC[n+]1ccccc1)OCCn1cnnn1 ; [Br-]] |
| HG3a-23 | 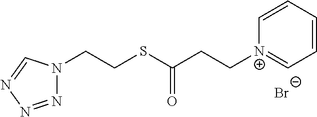 [SMILES: O=C(CC[n+]1ccccc1)SCCn1cnnn1 ; [Br-]] |
| HG3a-24 | 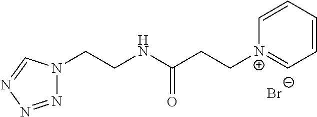 [SMILES: O=C(CC[n+]1ccccc1)NCCn1cnnn1 ; [Br-]] |
| HG3a-25 | 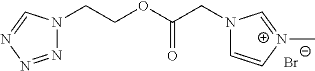 [SMILES: C[n+]1ccn(CC(=O)OCCn2cnnn2)c1 ; [Br-]] |
| HG3a-26 | 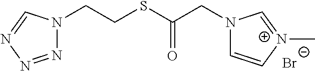 [SMILES: C[n+]1ccn(CC(=O)SCCn2cnnn2)c1 ; [Br-]] |
| HG3a-27 | 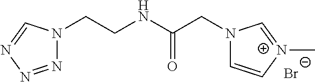 [SMILES: C[n+]1ccn(CC(=O)NCCn2cnnn2)c1 ; [Br-]] |
| HG3a-28 | 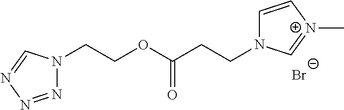 [SMILES: C[n+]1ccn(CCC(=O)OCCn2cnnn2)c1 ; [Br-]] |
| HG3a-29 | 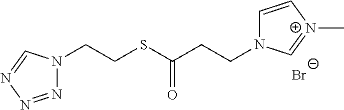 [SMILES: C[n+]1ccn(CCC(=O)SCCn2cnnn2)c1 ; [Br-]] |
| HG3a-30 | 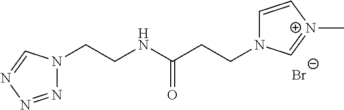 [SMILES: C[n+]1ccn(CCC(=O)NCCn2cnnn2)c1 ; [Br-]] |
| HG3a-31 | 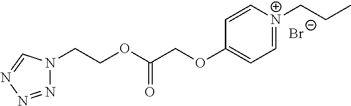 [SMILES: CCC[n+]1ccc(OCC(=O)OCCn2cnnn2)cc1 ; [Br-]] |
| HG3a-32 | 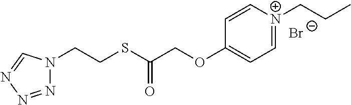 [SMILES: CCC[n+]1ccc(OCC(=O)SCCn2cnnn2)cc1 ; [Br-]] |
| HG3a-33 | 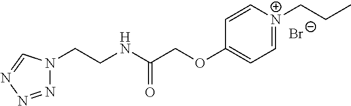 [SMILES: CCC[n+]1ccc(OCC(=O)NCCn2cnnn2)cc1 ; [Br-]] |
| HG3a-34 | 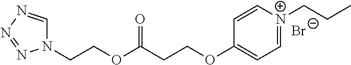 [SMILES: CCC[n+]1ccc(OCCC(=O)OCCn2cnnn2)cc1 ; [Br-]] |
| HG3a-35 | 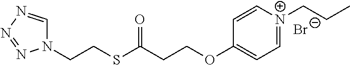 [SMILES: CCC[n+]1ccc(OCCC(=O)SCCn2cnnn2)cc1 ; [Br-]] |
| HG3a-36 | 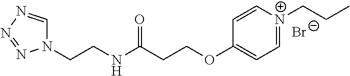 [SMILES: CCC[n+]1ccc(OCCC(=O)NCCn2cnnn2)cc1 ; [Br-]] |
| HG3a-37 | 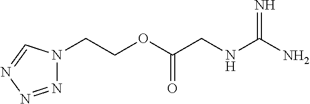 [SMILES: N=C(N)NCC(=O)OCCn1cnnn1] |
| HG3a-38 | 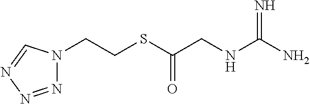 [SMILES: N=C(N)NCC(=O)SCCn1cnnn1] |
| HG3a-39 | 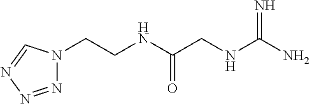 [SMILES: N=C(N)NCC(=O)NCCn1cnnn1] |
| HG3a-40 | 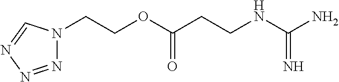 [SMILES: N=C(N)NCCC(=O)OCCn1cnnn1] |
| HG3a-41 | 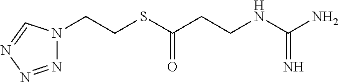 [SMILES: N=C(N)NCCC(=O)SCCn1cnnn1] |
| HG3a-42 | 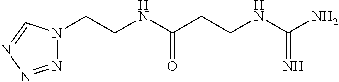 [SMILES: N=C(N)NCCC(=O)NCCn1cnnn1] |

TABLE 5
Compounds including the headgroup HG4a.

| Compound | Structure |
|---|---|
| HG4a-1 | 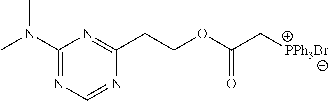 |
| HG4a-2 | 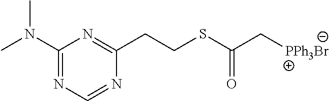 |
| HG4a-3 | 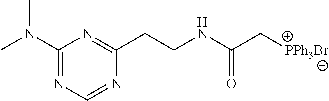 |
| HG4a-4 | 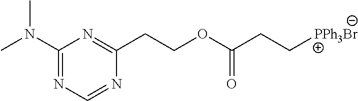 |
| HG4a-5 | 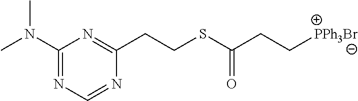 |
| HG4a-6 | 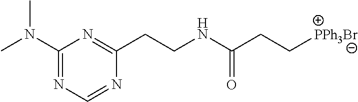 |
| HG4a-7 | 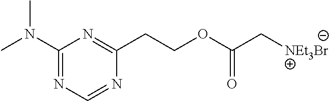 |
| HG4a-8 | 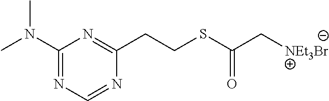 |
| HG4a-9 | 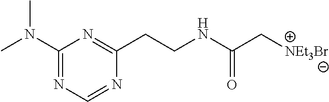 |
| HG4a-10 | 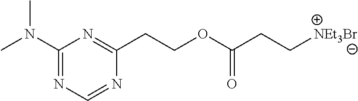 |
| HG4a-11 | 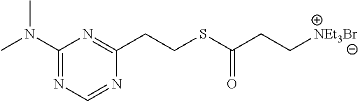 |
| HG4a-12 | 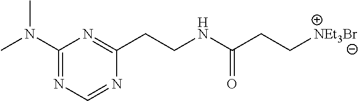 |
| HG4a-13 | 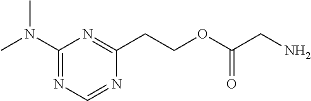 [SMILES: CN(C)c1ncnc(CCOC(=O)CN)n1] |
| HG4a-14 | 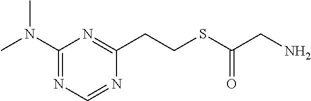 [SMILES: CN(C)c1ncnc(CCSC(=O)CN)n1] |
| HG4a-15 | 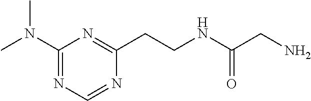 [SMILES: CN(C)c1ncnc(CCNC(=O)CN)n1] |
| HG4a-16 | 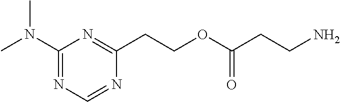 [SMILES: CN(C)c1ncnc(CCOC(=O)CCN)n1] |
| HG4a-17 | 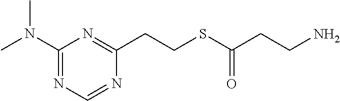 [SMILES: CN(C)c1ncnc(CCSC(=O)CCN)n1] |
| HG4a-18 | 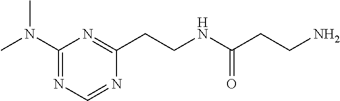 [SMILES: CN(C)c1ncnc(CCNC(=O)CCN)n1] |
| HG4a-19 | 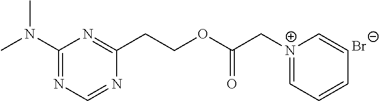 [SMILES: CN(C)c1ncnc(CCOC(=O)C[n+]2ccccc2)n1 ; [Br-]] |
| HG4a-20 | 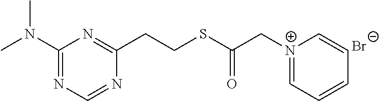 [SMILES: CN(C)c1ncnc(CCSC(=O)C[n+]2ccccc2)n1 ; [Br-]] |
| HG4a-21 | 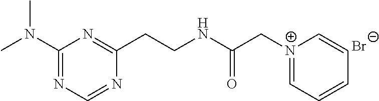 [SMILES: CN(C)c1ncnc(CCNC(=O)C[n+]2ccccc2)n1 ; [Br-]] |
| HG4a-22 | 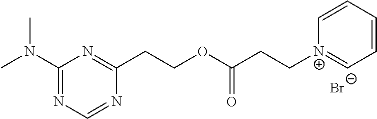 [SMILES: CN(C)c1ncnc(CCOC(=O)CC[n+]2ccccc2)n1 ; [Br-]] |
| HG4a-23 | 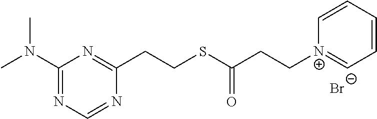 [SMILES: CN(C)c1ncnc(CCSC(=O)CC[n+]2ccccc2)n1 ; [Br-]] |
| HG4a-24 | 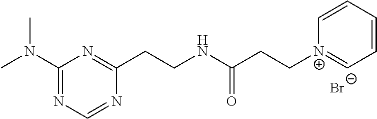 [SMILES: CN(C)c1ncnc(CCNC(=O)CC[n+]2ccccc2)n1 ; [Br-]] |
| HG4a-25 | 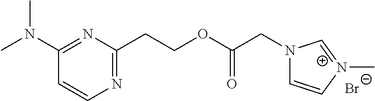 |
| HG4a-26 | 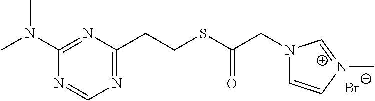 |
| HG4a-27 | 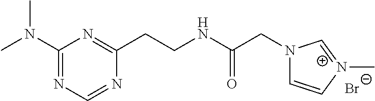 |
| HG4a-28 | 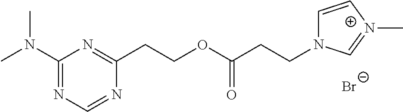 |
| HG4a-29 | 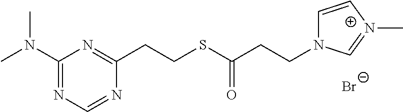 |
| HG5a-30 | 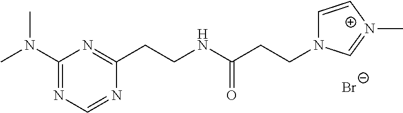 |
| HG4a-31 | 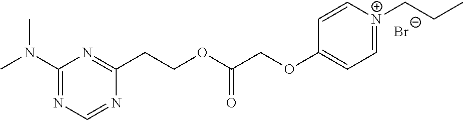 [SMILES: CCC[n+]1ccc(OCC(=O)OCCc2ncnc(N(C)C)n2)cc1 ; [Br-]] |
| HG4a-32 | 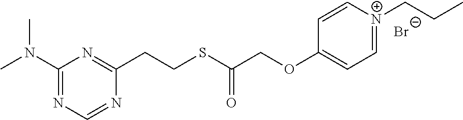 [SMILES: CCC[n+]1ccc(OCC(=O)SCCc2ncnc(N(C)C)n2)cc1 ; [Br-]] |
| HG4a-33 | 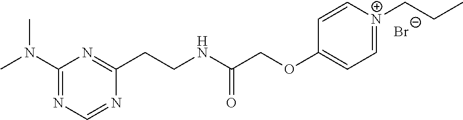 [SMILES: CCC[n+]1ccc(OCC(=O)NCCc2ncnc(N(C)C)n2)cc1 ; [Br-]] |
| HG4a-34 | 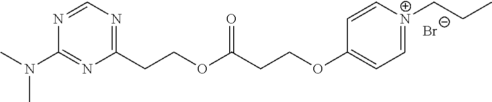 [SMILES: CCC[n+]1ccc(OCCC(=O)OCCc2ncnc(N(C)C)n2)cc1 ; [Br-]] |
| HG4a-35 | 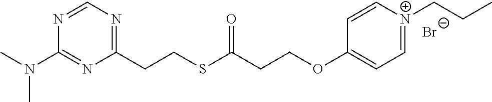 [SMILES: CCC[n+]1ccc(OCCC(=O)SCCc2ncnc(N(C)C)n2)cc1 ; [Br-]] |
| HG4a-36 | 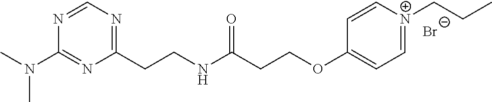 [SMILES: CCC[n+]1ccc(OCCC(=O)NCCc2ncnc(N(C)C)n2)cc1 ; [Br-]] |
| HG4a-37 | 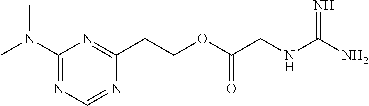 [SMILES: CN(C)c1ncnc(CCOC(=O)CNC(=N)N)n1] |
| HG4a-38 | 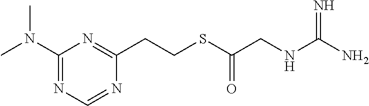 [SMILES: CN(C)c1ncnc(CCSC(=O)CNC(=N)N)n1] |
| HG4a-39 | 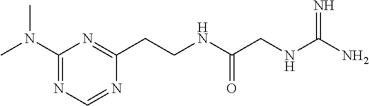 [SMILES: CN(C)c1ncnc(CCNC(=O)CNC(=N)N)n1] |
| HG4a-40 | 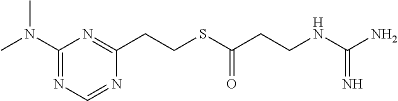 [SMILES: CN(C)c1ncnc(CCSC(=O)CCNC(=N)N)n1] |
| HG4a-41 | 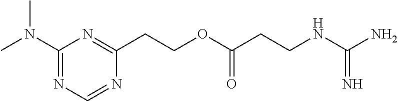 [SMILES: CN(C)c1ncnc(CCOC(=O)CCNC(=N)N)n1] |
| HG4a-42 | 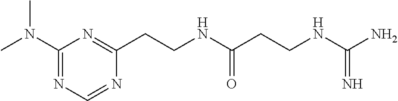 [SMILES: CN(C)c1ncnc(CCNC(=O)CCNC(=N)N)n1] |

TABLE 6
Compounds including the headgroup HG5a.

| Compound | Structure |
|---|---|
| HG5a-1 | 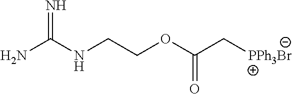 |
| HG5a-2 | 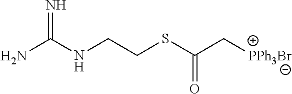 |
| HG5a-3 | 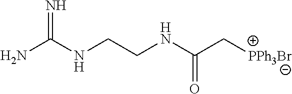 |
| HG5a-4 | 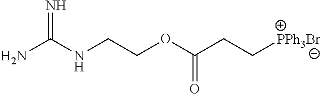 |
| HG5a-5 | 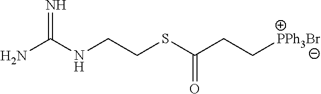 |
| HG5a-6 | 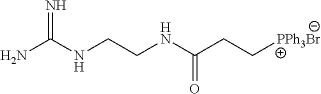 |
| HG5a-7 | 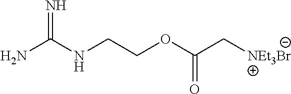 |
| HG5a-8 | 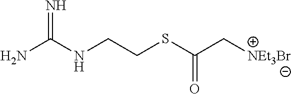 |
| HG5a-9 | 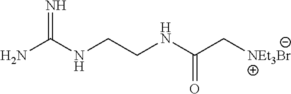 |
| HG5a-10 | 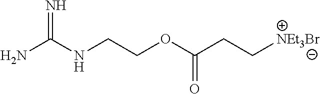 |
| HG5a-11 | 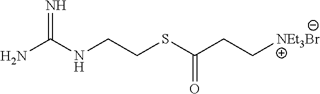 |
| HG5a-12 | 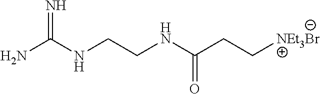 |
| HG5a-13 | 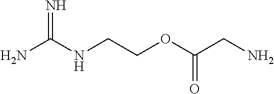 [SMILES: N=C(N)NCCOC(=O)CN] |
| HG5a-14 | 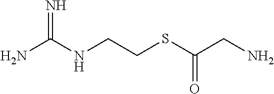 [SMILES: N=C(N)NCCSC(=O)CN] |
| HG5a-15 | 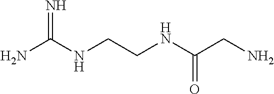 [SMILES: N=C(N)NCCNC(=O)CN] |
| HG5a-16 | 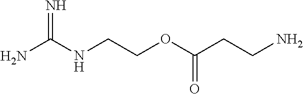 [SMILES: N=C(N)NCCOC(=O)CCN] |
| HG5a-17 | 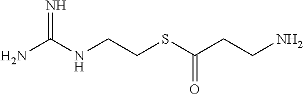 [SMILES: N=C(N)NCCSC(=O)CCN] |
| HG5a-18 | 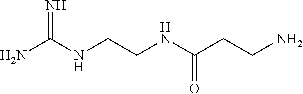 [SMILES: N=C(N)NCCNC(=O)CCN] |
| HG5a-19 | 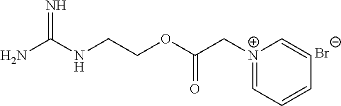 [SMILES: N=C(N)NCCOC(=O)C[n+]1ccccc1 ; [Br-]] |
| HG5a-20 | 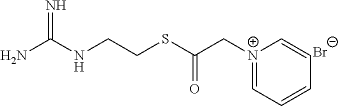 [SMILES: N=C(N)NCCSC(=O)C[n+]1ccccc1 ; [Br-]] |
| HG5a-21 | 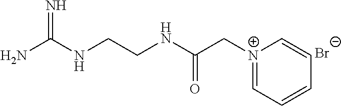 [SMILES: N=C(N)NCCNC(=O)C[n+]1ccccc1 ; [Br-]] |
| HG5a-22 | 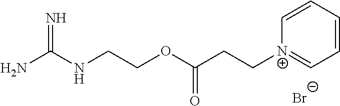 [SMILES: N=C(N)NCCOC(=O)CC[n+]1ccccc1 ; [Br-]] |
| HG5a-23 | 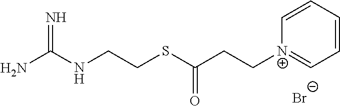 [SMILES: N=C(N)NCCSC(=O)CC[n+]1ccccc1 ; [Br-]] |
| HG5a-24 | 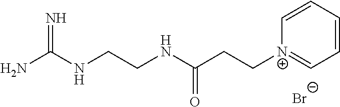 [SMILES: N=C(N)NCCNC(=O)CC[n+]1ccccc1 ; [Br-]] |
| HG5a-25 | 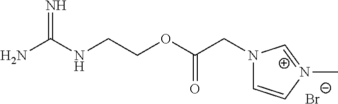 [SMILES: C[n+]1ccn(CC(=O)OCCNC(=N)N)c1 ; [Br-]] |
| HG5a-26 | 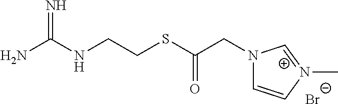 [SMILES: C[n+]1ccn(CC(=O)SCCNC(=N)N)c1 ; [Br-]] |
| HG5a-27 | 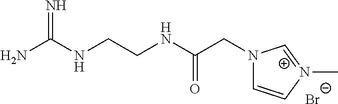 [SMILES: C[n+]1ccn(CC(=O)NCCNC(=N)N)c1 ; [Br-]] |
| HG5a-28 | 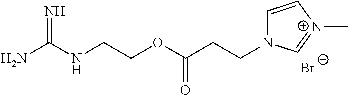 [SMILES: C[n+]1ccn(CCC(=O)OCCNC(=N)N)c1 ; [Br-]] |
| HG5a-29 | 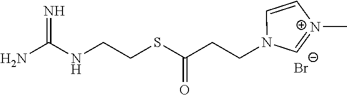 [SMILES: C[n+]1ccn(CCC(=O)SCCNC(=N)N)c1 ; [Br-]] |
| HG5a-30 | 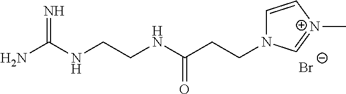 [SMILES: C[n+]1ccn(CCC(=O)NCCNC(=N)N)c1 ; [Br-]] |
| HG5a-31 | 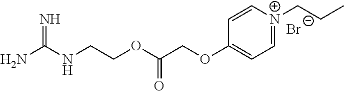 [SMILES: CCC[n+]1ccc(OCC(=O)OCCNC(=N)N)cc1 ; [Br-]] |
| HG5a-32 | 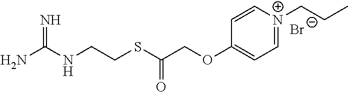 [SMILES: CCC[n+]1ccc(OCC(=O)SCCNC(=N)N)cc1 ; [Br-]] |
| HG5a-33 | 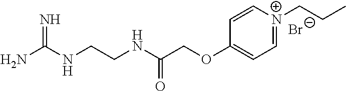 [SMILES: CCC[n+]1ccc(OCC(=O)NCCNC(=N)N)cc1 ; [Br-]] |
| HG5a-34 | 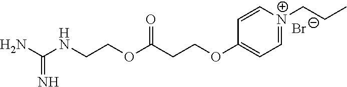 [SMILES: CCC[n+]1ccc(OCCC(=O)OCCNC(=N)N)cc1 ; [Br-]] |
| HG5a-35 | 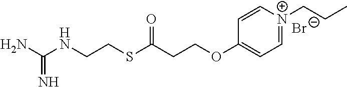 [SMILES: CCC[n+]1ccc(OCCC(=O)SCCNC(=N)N)cc1 ; [Br-]] |
| HG5a-36 | 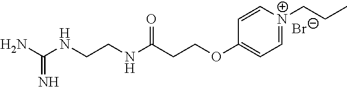 [SMILES: CCC[n+]1ccc(OCCC(=O)NCCNC(=N)N)cc1 ; [Br-]] |
| HG5a-37 | 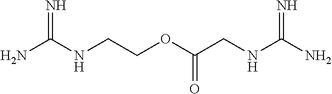 [SMILES: N=C(N)NCCOC(=O)CNC(=N)N] |
| HG5a-38 | 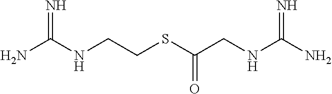 [SMILES: N=C(N)NCCSC(=O)CNC(=N)N] |
| HG5a-39 | 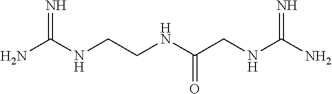 [SMILES: N=C(N)NCCNC(=O)CNC(=N)N] |
| HG5a-40 | 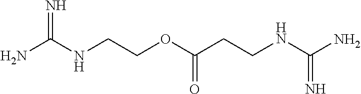 [SMILES: N=C(N)NCCOC(=O)CCNC(=N)N] |
| HG5a-41 | 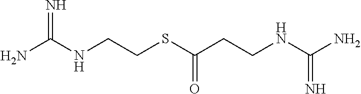 [SMILES: N=C(N)NCCSC(=O)CCNC(=N)N] |
| HG5a-42 | 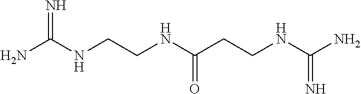 [SMILES: N=C(N)NCCNC(=O)CCNC(=N)N] |

TABLE 7
Compounds including the headgroup HG6a.

| Compound | Structure |
|---|---|
| HG6a-1 | 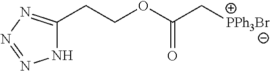 |
| HG6a-2 | 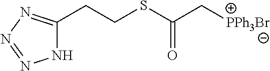 |
| HG6a-3 | 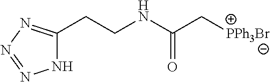 |
| HG6a-4 | 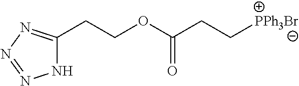 |
| HG6a-5 | 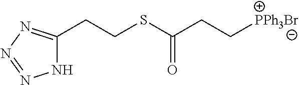 |
| HG6a-6 | 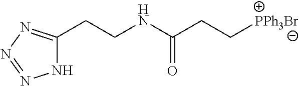 |
| HG6a-7 | 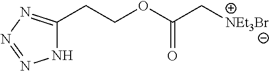 |
| HG6a-8 | 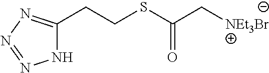 |
| HG6a-9 | 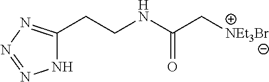 |
| HG6a-10 | 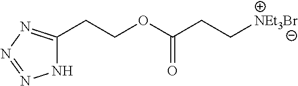 |
| HG6a-11 | 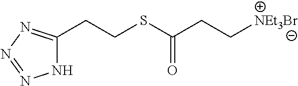 |
| HG6a-12 | 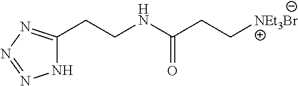 |
| HG6a-13 | 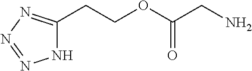 [SMILES: NCC(=O)OCCc1nnn[nH]1] |
| HG6a-14 | 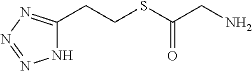 [SMILES: NCC(=O)SCCc1nnn[nH]1] |
| HG6a-15 | 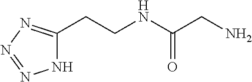 [SMILES: NCC(=O)NCCc1nnn[nH]1] |
| HG6a-16 | 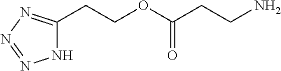 [SMILES: NCCC(=O)OCCc1nnn[nH]1] |
| HG6a-17 | 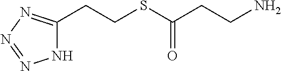 [SMILES: NCCC(=O)SCCc1nnn[nH]1] |
| HG6a-18 | 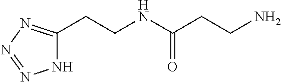 [SMILES: NCCC(=O)NCCc1nnn[nH]1] |
| HG6a-19 | 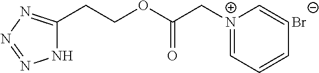 [SMILES: O=C(C[n+]1ccccc1)OCCc1nnn[nH]1 ; [Br-]] |
| HG6a-20 | 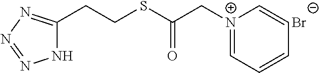 [SMILES: O=C(C[n+]1ccccc1)SCCc1nnn[nH]1 ; [Br-]] |
| HG6a-21 | 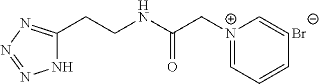 [SMILES: O=C(C[n+]1ccccc1)NCCc1nnn[nH]1 ; [Br-]] |
| HG6a-22 | 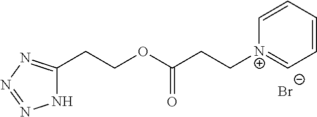 [SMILES: O=C(CC[n+]1ccccc1)OCCc1nnn[nH]1 ; [Br-]] |
| HG6a-23 | 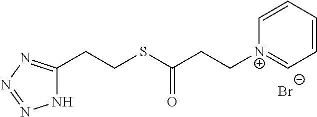 [SMILES: O=C(CC[n+]1ccccc1)SCCc1nnn[nH]1 ; [Br-]] |
| HG6a-24 | 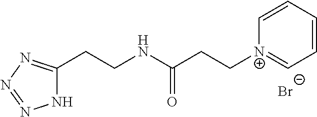 [SMILES: O=C(CC[n+]1ccccc1)NCCc1nnn[nH]1 ; [Br-]] |
| HG6a-25 | 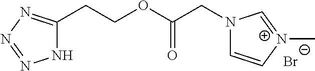 [SMILES: C[n+]1ccn(CC(=O)OCCc2nnn[nH]2)c1 ; [Br-]] |
| HG6a-26 | 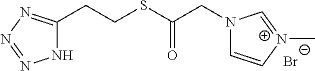 [SMILES: C[n+]1ccn(CC(=O)SCCc2nnn[nH]2)c1 ; [Br-]] |
| HG6a-27 | 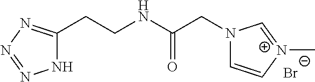 [SMILES: C[n+]1ccn(CC(=O)NCCc2nnn[nH]2)c1 ; [Br-]] |
| HG6a-28 | 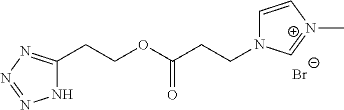 [SMILES: C[n+]1ccn(CCC(=O)OCCc2nnn[nH]2)c1 ; [Br-]] |
| HG6a-29 | 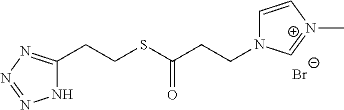 [SMILES: C[n+]1ccn(CCC(=O)SCCc2nnn[nH]2)c1 ; [Br-]] |
| HG6a-30 | 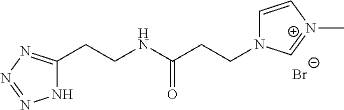 [SMILES: C[n+]1ccn(CCC(=O)NCCc2nnn[nH]2)c1 ; [Br-]] |
| HG6a-31 | 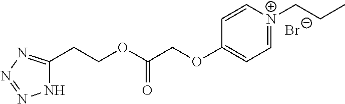 [SMILES: CCC[n+]1ccc(OCC(=O)OCCc2nnn[nH]2)cc1 ; [Br-]] |
| HG6a-32 | 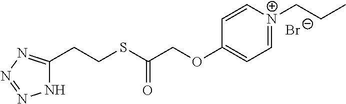 [SMILES: CCC[n+]1ccc(OCC(=O)SCCc2nnn[nH]2)cc1 ; [Br-]] |
| HG6a-33 | 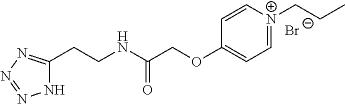 [SMILES: CCC[n+]1ccc(OCC(=O)NCCc2nnn[nH]2)cc1 ; [Br-]] |
| HG6a-34 | 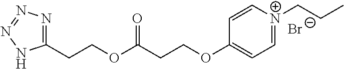 [SMILES: CCC[n+]1ccc(OCCC(=O)OCCc2nnn[nH]2)cc1 ; [Br-]] |
| HG6a-35 | 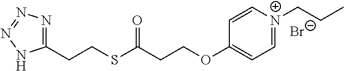 [SMILES: CCC[n+]1ccc(OCCC(=O)SCCc2nnn[nH]2)cc1 ; [Br-]] |
| HG6a-36 | 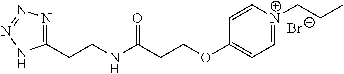 [SMILES: CCC[n+]1ccc(OCCC(=O)NCCc2nnn[nH]2)cc1 ; [Br-]] |
| HG6a-37 | 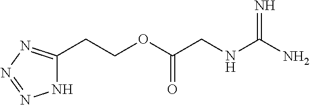 [SMILES: N=C(N)NCC(=O)OCCc1nnn[nH]1] |
| HG6a-38 | 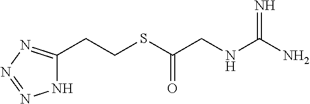 [SMILES: N=C(N)NCC(=O)SCCc1nnn[nH]1] |
| HG6a-39 | 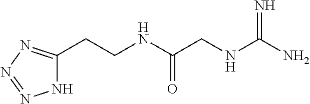 [SMILES: N=C(N)NCC(=O)NCCc1nnn[nH]1] |
| HG6a-40 | 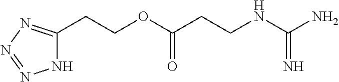 [SMILES: N=C(N)NCCC(=O)OCCc1nnn[nH]1] |
| HG6a-41 | 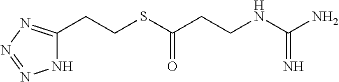 [SMILES: N=C(N)NCCC(=O)SCCc1nnn[nH]1] |
| HG6a-42 | 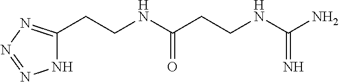 [SMILES: N=C(N)NCCC(=O)NCCc1nnn[nH]1] |

TABLE 8
Compounds including the headgroup HG7a.

| Compound | Structure |
|---|---|
| HG7a-1 | 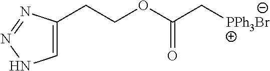 |
| HG7a-2 | 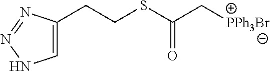 |
| HG7a-3 | 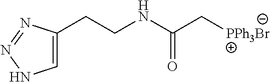 |
| HG7a-4 | 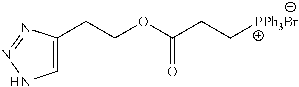 |
| HG7a-5 | 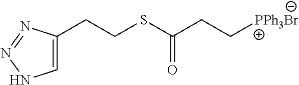 |
| HG7a-6 | 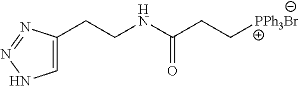 |
| HG7a-7 | 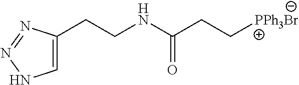 |
| HG7a-8 | 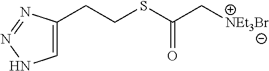 |
| HG7a-9 | 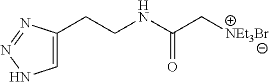 |
| HG7a-10 | 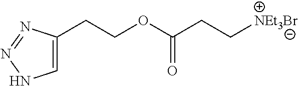 |
| HG7a-11 | 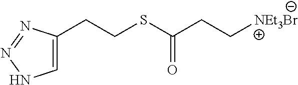 |
| HG7a-12 | 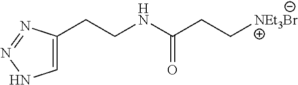 |
| HG7a-13 | 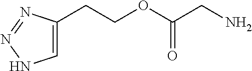 [SMILES: NCC(=O)OCCc1c[nH]nn1] |
| HG7a-14 | 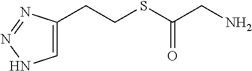 [SMILES: NCC(=O)SCCc1c[nH]nn1] |
| HG7a-15 | 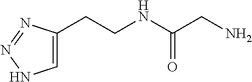 [SMILES: NCC(=O)NCCc1c[nH]nn1] |
| HG7a-16 | 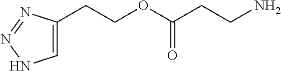 [SMILES: NCCC(=O)OCCc1c[nH]nn1] |
| HG7a-17 | 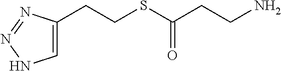 [SMILES: NCCC(=O)SCCc1c[nH]nn1] |
| HG7a-18 | 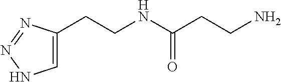 [SMILES: NCCC(=O)NCCc1c[nH]nn1] |
| HG7a-19 | 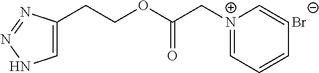 [SMILES: O=C(C[n+]1ccccc1)OCCc1c[nH]nn1 ; [Br-]] |
| HG7a-20 | 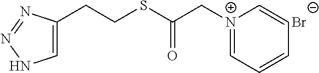 [SMILES: O=C(C[n+]1ccccc1)SCCc1c[nH]nn1 ; [Br-]] |
| HG7a-21 | 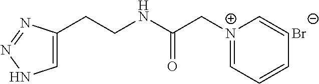 [SMILES: O=C(C[n+]1ccccc1)NCCc1c[nH]nn1 ; [Br-]] |
| HG7a-22 | 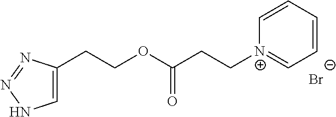 [SMILES: O=C(CC[n+]1ccccc1)OCCc1c[nH]nn1 ; [Br-]] |
| HG7a-23 | 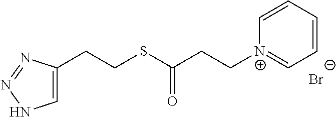 [SMILES: O=C(CC[n+]1ccccc1)SCCc1c[nH]nn1 ; [Br-]] |
| HG7a-24 | 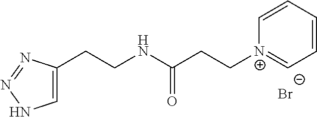 [SMILES: O=C(CC[n+]1ccccc1)NCCc1c[nH]nn1 ; [Br-]] |
| HG7a-25 | 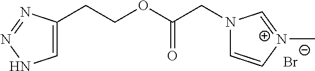 [SMILES: C[n+]1ccn(CC(=O)OCCc2c[nH]nn2)c1 ; [Br-]] |
| HG7a-26 | 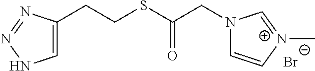 [SMILES: C[n+]1ccn(CC(=O)SCCc2c[nH]nn2)c1 ; [Br-]] |
| HG7a-27 | 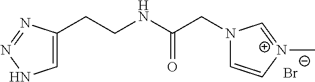 [SMILES: C[n+]1ccn(CC(=O)NCCc2c[nH]nn2)c1 ; [Br-]] |
| HG7a-28 | 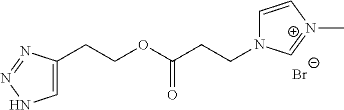 [SMILES: C[n+]1ccn(CCC(=O)OCCc2c[nH]nn2)c1 ; [Br-]] |
| HG7a-29 | 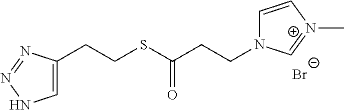 [SMILES: C[n+]1ccn(CCC(=O)SCCc2c[nH]nn2)c1 ; [Br-]] |
| HG7a-30 | 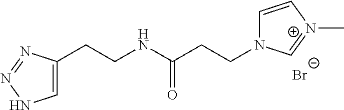 [SMILES: C[n+]1ccn(CCC(=O)NCCc2c[nH]nn2)c1 ; [Br-]] |
| HG7a-31 | 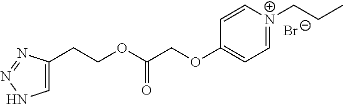 [SMILES: CCC[n+]1ccc(OCC(=O)OCCc2c[nH]nn2)cc1 ; [Br-]] |
| HG7a-32 | 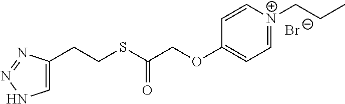 [SMILES: CCC[n+]1ccc(OCC(=O)SCCc2c[nH]nn2)cc1 ; [Br-]] |
| HG7a-33 | 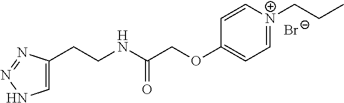 [SMILES: CCC[n+]1ccc(OCC(=O)NCCc2c[nH]nn2)cc1 ; [Br-]] |
| HG7a-34 | 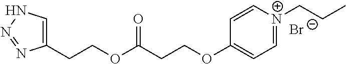 [SMILES: CCC[n+]1ccc(OCCC(=O)OCCc2c[nH]nn2)cc1 ; [Br-]] |
| HG7a-35 | 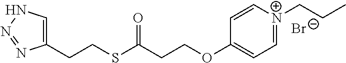 [SMILES: CCC[n+]1ccc(OCCC(=O)SCCc2c[nH]nn2)cc1 ; [Br-]] |
| HG7a-36 | 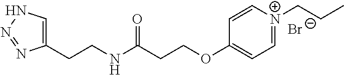 [SMILES: CCC[n+]1ccc(OCCC(=O)NCCc2c[nH]nn2)cc1 ; [Br-]] |
| HG7a-37 | 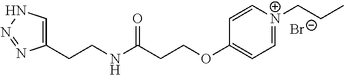 [SMILES: CCC[n+]1ccc(OCCC(=O)NCCc2c[nH]nn2)cc1 ; [Br-]] |
| HG7a-38 | 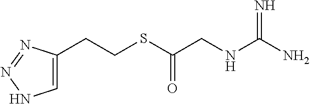 [SMILES: N=C(N)NCC(=O)SCCc1c[nH]nn1] |
| HG7a-39 | 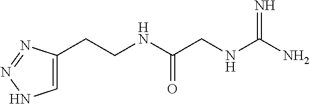 [SMILES: N=C(N)NCC(=O)NCCc1c[nH]nn1] |
| HG7a-40 | 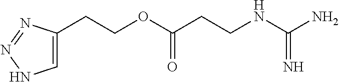 [SMILES: N=C(N)NCCC(=O)OCCc1c[nH]nn1] |
| HG7a-41 | 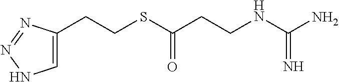 [SMILES: N=C(N)NCCC(=O)SCCc1c[nH]nn1] |
| HG7a-42 | 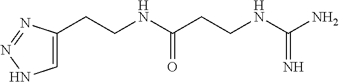 [SMILES: N=C(N)NCCC(=O)NCCc1c[nH]nn1] |

In certain embodiments, the compound is selected from any of compounds (A1)-(A15):

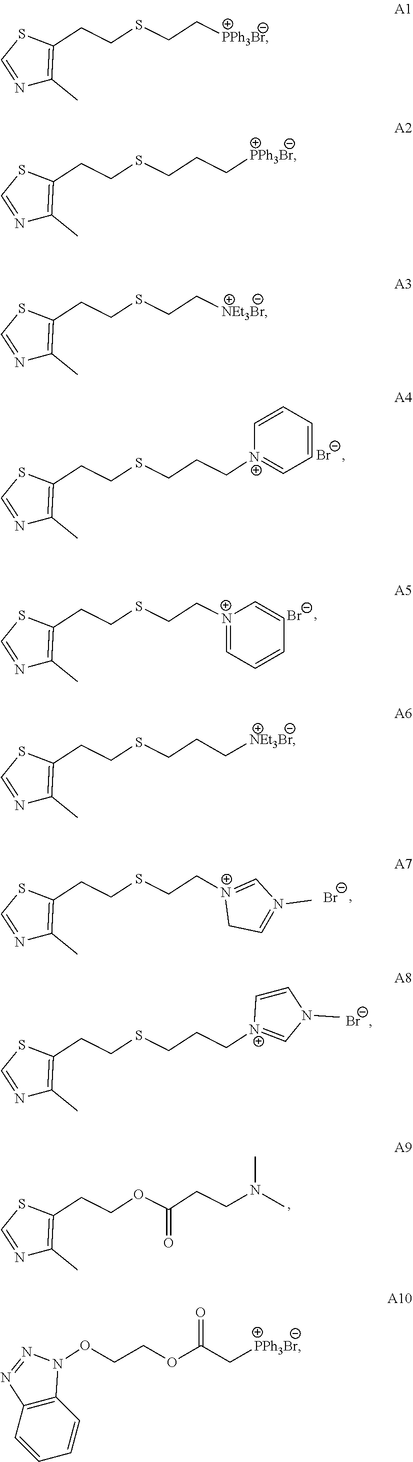

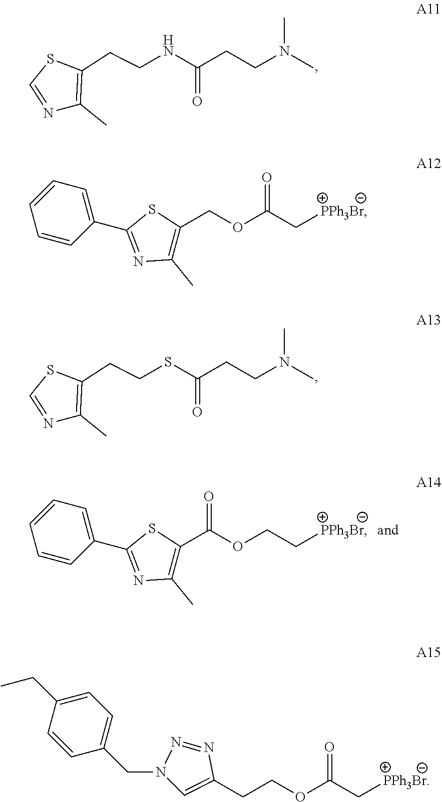

In certain embodiments, the compound is selected from any of compounds (HG1a-49)-(A32):

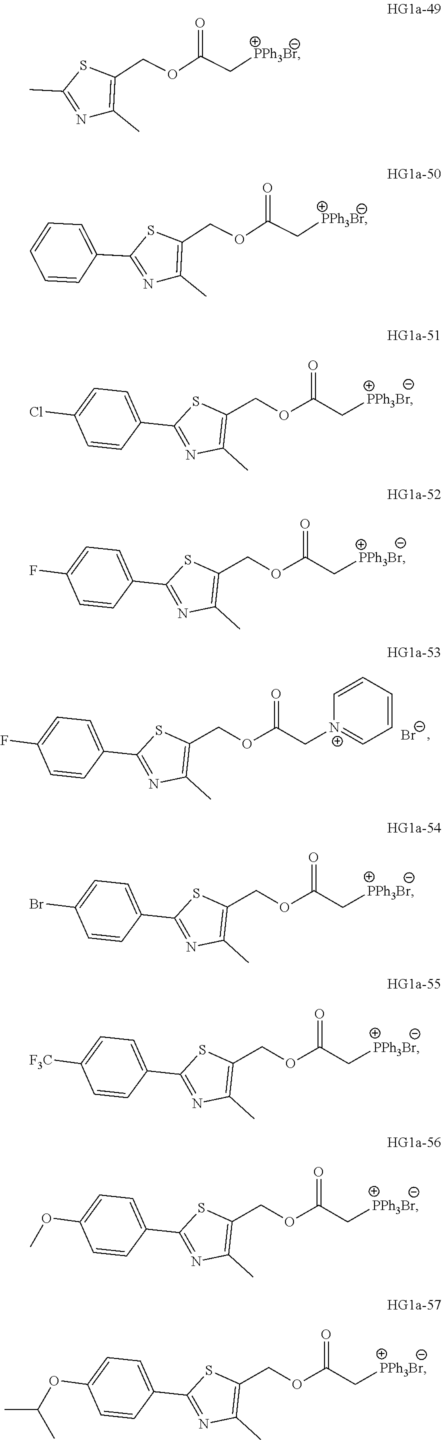

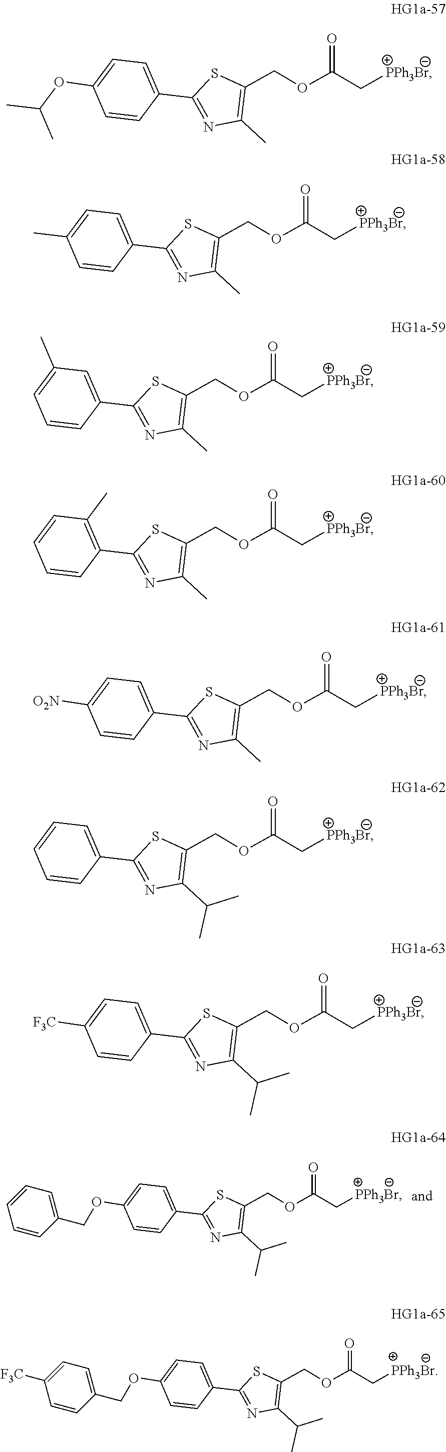

In certain embodiments, the compound is HG1a-66:

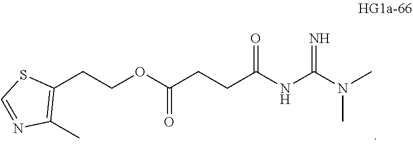 [SMILES: Cc1ncsc1CCOC(=O)CCC(=O)NC(=N)N(C)C]

In certain embodiments, the compound is selected from any of compounds (HG1b-1)-(HG1b-6):

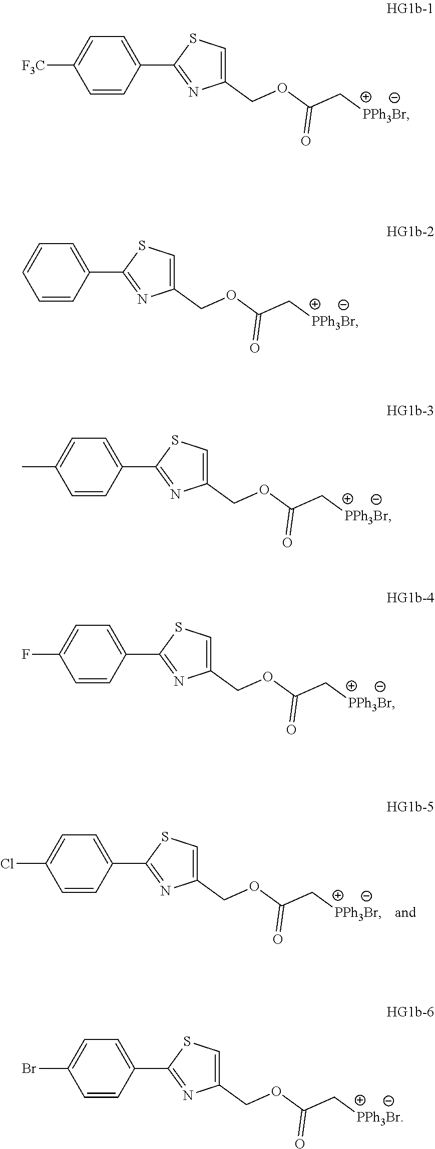

Aspects of the present disclosure include the subject compounds, salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject compounds, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-I,4-dioate, hexyne-I,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). In some cases, the promoiety is attached to a hydroxy or carboxylic acid group of the subject compounds. In certain cases, the promoiety is an acyl or substituted acyl group. In certain cases, the promoiety is an alkyl or substituted alkyl group, e.g., that forms an ester functional group when attached to a carboxylic acid group of the subject compounds.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the subject compounds are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability.

In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

Methods

As summarized above, aspects of the disclosure include mitochondrial modulator compounds, and methods of treatment using the same. Mitochondrial moderation or inhibition can improve markers mitochondrial health, which leads to improved phenotypes in metabolic syndrome-related diseases (e.g., as described herein).

Aspects of the subject methods include a method of modulating mitochondria (e.g., moderating or inhibiting mitochondria). Aspects of the subject methods include treating a subject having a metabolic syndrome-related disease or a symptom thereof by administering to the subject a therapeutically effective amount of a subject compound. In certain cases, the disease is selected from hyperlipidemia, type 2 diabetes, fatty liver disease, obesity, cardiovascular disease and stroke. In certain cases, the symptom is selected from abdominal obesity, insulin resistance, hyperinsulinemia, high levels of blood fats, increased blood pressure, and elevated serum lipids.

Mitochondrial Modulation

Aspects of the invention include mitochondrial modulator compounds that can moderate or inhibit mitochondria. In some cases, the moderation or inhibition is reversible. In some cases, the subject compound can modulate cytochrome c oxidase complex IV. The cytochrome c oxidase complex IV spans the inner mitochondrial membrane. It is the terminal oxidase of the respiratory chain in the transfer of electrons from cytochrome c to oxygen. Cytochrome c is not an integral part of complex IV, but is stoichiometrically associated with it and is believed to be spatially associated with subunit II of cytochrome oxidase. Cytochrome c is a water-soluble electron carrier and exists between the internal and external mitochondrial membranes. It can diffuse freely in this space, thus acting as a mobile shuttle carrying electrons between cytochrome c1 of complex III and cytochrome a of complex IV. CcO is a highly regulated enzyme which is believed to be the pace setter for mitochondrial oxidative metabolism and ATP synthesis.

By inhibiting mitochondria it is meant that the activity of a mitochondrial enzyme is decreased by 10% or more, such as 20% or more, 30% or more, 40% or more, 50% or more, 60% or more, 70% or more, 80% or more, 90% or more, 95% or more (e.g., relative to a control in any convenient in vitro inhibition assay). In some cases, inhibiting mitochondria means decreasing the activity of a mitochondrial enzyme by a factor of 2 or more, such as 3 or more, 5 or more, 10 or more, 100 or more, or 1000 or more, relative to its normal activity (e.g., relative to a control as measured by any convenient assay).

In some cases, the method is a method of mitochondrial modulation in a sample. The term “sample” as used herein relates to a material or mixture of materials, typically, although not necessarily, in fluid form, containing one or more components of interest.

In some embodiments, there is provided a method of modulating mitochondria, the method comprising contacting a sample with a subject compound to modulate the activity of a mitochondria enzyme. In some cases, the sample is a cellular sample.

In certain embodiments the subject compound is a compound as defined herein. In some embodiments, the compound is a compound according to any one of formulas (I)-(IE). In some cases, the subject compound is any one of compounds described in Tables 1-7, or compounds A1-A15.

In some embodiments the subject compound is cell permeable. In some embodiments, there is provided a method of moderating mitochondria, the method comprising contacting a sample with a cell permeable compound. CcO.activity may be modulated by the compound.

In some embodiments, the subject compounds have a mitochondrial inhibition profile that reflects activity against additional enzymes. In some embodiments, the subject compounds can specifically inhibit a mitochondrial enzyme without undesired inhibition of one or more other enzymes.

In some embodiments, the subject compounds have a mitochondrial inhibition profile that reflects activity against additional enzymes. In some embodiments, the subject compounds may specifically inhibit an unknown target that reduces the activity of CcO in some cases without undesired inhibition of one or more other enzymes.

In some embodiments, the subject compounds inhibit mitochondria, as determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting mitochondria. A subject compound (e.g., as described herein) may inhibit at activity of mitochondria in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10⁻⁶ M or less (e.g., 1×10⁻⁶ M or less, 1×10⁻⁷ M or less, 1×10⁻⁸ M or less, 1×10⁻⁹ M or less, 1×10⁻¹⁰ M or less, or 1×10⁻¹¹ M or less).

The protocols that may be employed in determining mitochondrial activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition related to the target pathogen).

In some embodiments, the subject method is an in vitro method that includes contacting a sample with a subject compound that specifically modulates mitochondria.

In certain embodiments, the sample is suspected of containing a mitochondrial enzyme and the subject method further comprises evaluating whether the compound modulates the mitochondrial enzyme.

In certain embodiments, the subject compound is a modified compound that includes a label, e.g., a fluorescent label, and the subject method further includes detecting the label, if present, in the sample, e.g., using optical detection.

In certain embodiments, the compound is modified with a support or with affinity groups that bind to a support (e.g. biotin), such that any sample that does not bind to the compound may be removed (e.g., by washing). The specifically bound mitochondrial enzyme, if present, may then be detected using any convenient means, such as, using the binding of a labeled target specific probe, or using a fluorescent protein reactive reagent.

In another embodiment of the subject method, the sample is known to contain a mitochondrial enzyme.

In some embodiments, the method is a method of treating a metabolic syndrome-related disease, where the method includes contacting the cell with an effective amount of a subject compound (e.g., as described herein) to treat the metabolic syndrome-related disease, or a symptom thereof. In certain cases, the subject compounds can act intracellularly. The method can be performed in combination with a second therapeutic agent (e.g., as described herein). The target cells can be in vitro or in vivo.

Methods of Treating a Metabolic Syndrome-Related Disease

The present disclosure provides methods for treating or preventing metabolic syndrome-related diseases, such as, hyperlipidemia, type 2 diabetes, fatty liver diseases, cardiovascular disease, stroke, obesity and other body weight disorders, hyperglycemia, hyperinsulinemia, glucose intolerance, and glucose metabolism disorders, by the administration of the subject compounds, or compositions thereof, as described herein. Such methods may also have an advantageous effect on one or more symptoms associated with a metabolic syndrome related-disease, disorder or condition by, for example, decreasing the severity or the frequency of a symptom. In certain embodiments, the symptom is selected from abdominal obesity, insulin resistance, hyperinsulinemia, high levels of blood fats, increased blood pressure, and elevated serum lipids. In specific embodiments, the present disclosure provides methods for treating a glucose metabolism or body weight disorder by the administration of the subject compounds or compositions thereof. In particular embodiment, the present disclosure methods for decreasing body weight by the administration of the subject compounds or compositions thereof. The present disclosure further provides a use of any of the subject compounds or compositions thereof in the manufacture of a medicament for use in treating a metabolic syndrome-related disease selected from hyperlipidemia, type 2 diabetes, fatty liver diseases, cardiovascular disease, stroke, obesity and other body weight disorders, hyperglycemia, hyperinsulinemia, glucose intolerance, and glucose metabolism disorders. The present disclosure further provides a use of any of the subject compounds or compositions thereof in the manufacture of a medicament for use in treating a glucose metabolism or body weight disorder. The present disclosure further provides a use of the subject compounds or compositions thereof in the manufacture of a medicament for use in decreasing body weight. In certain embodiments, the subject methods offer a convenient alternative to calorie reduction.

In order to determine whether a subject may be a candidate for the treatment or prevention of a body weight disorder (e.g., obesity) by the methods provided herein, parameters such as, but not limited to, the etiology and the extent of the subject's condition (e.g., how overweight the subject is compared to reference healthy individual) should be evaluated. For example, an adult having a BMI between ˜25 and ˜29.9 kg/m2 may be considered overweight (pre-obese), while an adult having a BMI of ˜30 kg/m2 or higher may be considered obese.

In order to determine whether a subject may be a candidate for the treatment or prevention of hyperglycemia, hyperinsulinemia, glucose intolerance, and/or glucose disorders by the methods provided herein, various diagnostic methods known in the art may be utilized. Such methods include those described elsewhere herein (e.g., fasting plasma glucose (FPG) evaluation and the oral glucose tolerance test (oGTT)).

The compounds and compositions thereof provided herein when administered to a subject for treating or preventing a metabolic syndrome-related disease, such as, hyperlipidemia, type 2 diabetes, fatty liver diseases, cardiovascular disease, stroke, obesity and other body weight disorders, hyperglycemia, hyperinsulinemia, glucose intolerance, and glucose metabolism disorder, may lead to a reduction in blood glucose level, a reduction in body weight, a reduction in markers of DNA damage, a reduction in markers of inflammation, and a reduction of reactive oxygen species.

In certain embodiments, the subject compound or composition contemplated herein may decrease one or more of blood glucose level, body weight, markers of DNA damage, markers of inflammation, reactive oxygen species, by at least 5% compared to that in the absence of administration of a subject compound. For example, compounds and compositions contemplated herein may decrease one or more of blood glucose level, body weight, markers of DNA damage, markers of inflammation, reactive oxygen species, by at least 10%, 20%, 30%, 50%, 60%, 70%, 80%, or 90% as compared to that prior to the start of the treatment or prevention.

In some cases, the method is a method of modulating mitochondrial activity in a sample. As such, aspects of the method include contacting a sample with a subject compound (e.g., as described above) under conditions by which the compound modulates mitochondrial activity in the sample. Any convenient protocol for contacting the compound with the sample may be employed. The particular protocol that is employed may vary, e.g., depending on whether the sample is in vitro or in vivo. For in vitro protocols, contact of the sample with the compound may be achieved using any convenient protocol. In some instances, the sample includes cells that are maintained in a suitable culture medium, and the complex is introduced into the culture medium. For in vivo protocols, any convenient administration protocol may be employed. Depending upon the potency of the compound, the cells of interest, the manner of administration, the number of cells present, various protocols may be employed.

The term “sample” as used herein relates to a material or mixture of materials, typically, although not necessarily, in fluid form, containing one or more components of interest.

In some embodiments, the subject method is a method of treating a subject for a metabolic syndrome-related disease or disorder (e.g., as described herein). In some embodiments, the subject method includes administering to the subject an effective amount of a subject compound (e.g., as described herein) or a pharmaceutically acceptable salt thereof. The subject compound may be administered as part of a pharmaceutical composition (e.g., as described herein). In certain instances of the method, the compound that is administered is a compound of one of formulae (I)-(IE). In certain instances of the method, the compound that is administered is described by one of the compounds of Tables 1-7 or any one of compounds A1-A15.

In some embodiments, an effective amount of a subject compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a subject compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg or about 3000 mg

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw), or once pert week (qw) over a period of time. For example, a compound is administered qid, qd, qod, qw, tiw, or biw over a period of from one day to about 2 years or more. For example, a compound is administered at any of the aforementioned frequencies for one week, two weeks, one month, two months, six months, one year, or two years, or more, depending on various factors.

Any of a variety of methods can be used to determine whether a treatment method is effective. For example, a biological sample obtained from an individual who has been treated with a subject method can be assayed.

In some embodiments, the subject is a mammal. In some cases, the subject is a human. The subject may be in need of treatment for a metabolic syndrome-related disease, or may be at risk of a metabolic syndrome-related disease or disorder. In some instances, the subject methods include diagnosing a metabolic syndrome-related disease or disorder, including any one of the diseases or disorders described herein. In some embodiments, the compound is administered as a pharmaceutical preparation.

In some embodiments, the subject method is a method of modulating mitochondrial activity, the method including contacting cells with an effective dose of a subject compound (e.g., as described above). CcO activity may be modulated in this method. In some embodiments, the method further includes contacting the cells with a second active agent (e.g., as described herein).

In certain embodiments, the subject compound is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p 977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabeled and a secondary labeling agent is used for imaging.

### Combination Therapies

The subject compounds disclosed herein can be administered to a subject alone or in combination with an additional, i.e., second, active agent. Combination therapeutic methods where the subject compounds may be used in combination with a second active agent or an additional therapy, e.g., radiation therapy. The terms “agent,” “compound,” and “drug” are used interchangeably herein. For example, the subject compounds can be administered alone or in conjunction with one or more other drugs, such as drugs employed in the treatment of diseases of interest, including but not limited to, Metabolic-syndrome-related diseases. In some embodiments, the subject method further includes co-administering concomitantly or in sequence a second agent, e.g., a small molecule, a chemotherapeutic, an antibody, an antibody fragment, an antibody-drug conjugate, an aptamer, a protein, or a checkpoint inhibitor. In some embodiments, the method further includes performing radiation therapy on the subject.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug or additional therapy with a pharmaceutical composition of the present disclosure means administration of the compound and second agent or additional therapy at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs or therapies and compounds of the present disclosure.

In some embodiments, the compounds (e.g., a subject compound and the at least one additional compound or therapy) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

The compounds of the present disclosure can be used in combination with other agents useful in the treatment, prevention, suppression or amelioration of the diseases, disorders or conditions set forth herein, including but not limited to, those that are normally administered to subjects suffering from obesity, eating disorder, hyperglycemia, hyperinsulinemia, glucose intolerance, other glucose metabolism disorders, fatty liver disease, cardiovascular disease and or stroke.

The present disclosure contemplates combination therapy with numerous agents (and classes thereof), including 1) insulin, insulin mimetics and agents that entail stimulation of insulin secretion, including sulfonylureas (e.g., chlorpropamide, tolazamide, acetohexamide, tolbutamide, glyburide, glimepiride, glipizide) and meglitinides (e.g., mitiglinide, repaglinide (PRANDIN) and nateglinide (STARLIX)); 2) biguanides (e.g., metformin (GLUCOPHAGE), and its pharmaceutically acceptable salts, in particular, metformin hydrochloride, and extended-release formulations thereof, such as Glumetza™, Fortamet™, and GlucophageXR™) and other agents that act by promoting glucose utilization, reducing hepatic glucose production and/or diminishing intestinal glucose output; 3) alpha-glucosidase inhibitors (e.g., acarbose, voglibose and miglitol) and other agents that slow down carbohydrate digestion and consequently absorption from the gut and reduce postprandial hyperglycemia; 4) thiazolidinediones (e.g., rosiglitazone (AVANDIA), troglitazone (REZULIN), pioglitazone (ACTOS), glipizide, balaglitazone, rivoglitazone, netoglitazone, AMG 131, MBX2044, mitoglitazone, lobeglitazone, IDR-105, troglitazone, englitazone, ciglitazone, adaglitazone, darglitazone that enhance insulin action (e.g., by insulin sensitization) including insulin, and insulin mimetics (e.g., insulin degludec, insulin glargine, insulin lispro, insulin detemir, insulin glulisine and inhalable formulations of each), thus promoting glucose utilization in peripheral tissues; 5) glucagon-like-peptides including DPP-IV inhibitors (e.g., alogliptin, omarigliptin, linagliptin, vildagliptin (GALVUS) and sitagliptin (JANUVIA)) and Glucagon-Like Peptide-1 (GLP-1) and GLP-1 agonists and analogs (e.g., exenatide (BYETTA and ITCA 650 (an osmotic pump inserted subcutaneously that delivers an exenatide analog over a 12-month period; Intarcia, Boston, Mass.)) and GLP-1 receptor agonists (e.g., dulaglutide, semaglutide, albiglutide, exenatide, liraglutide, lixisenatide, taspoglutide, CJC-1131, and BIM-51077, including intranasal, transdermal, and once-weekly formulations thereof); 6) and DPP-IV-resistant analogues (incretin mimetics), PPAR gamma agonists, PPAR alpha agonists such as fenofibric acid derivatives (e.g., gemfibrozil, clofibrate, ciprofibrate, fenofibrate, bezafibrate), dual-acting PPAR agonists (e.g., ZYH2, ZYH1, GFT505, chiglitazar, muraglitazar, aleglitazar, sodelglitazar, and naveglitazar), pan-acting PPAR agonists, PTP1B inhibitors (e.g., ISIS-113715 and TTP814), SGLT inhibitors (e.g., ASP1941, SGLT-3, empagliflozin, dapagliflozin, canagliflozin, BI-10773, PF-04971729, remogloflozin, TS-071, tofogliflozin, ipragliflozin, and LX-4211), insulin secretagogues, angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan i.e., COZAAR®, valsartan, candesartan, olmesartan, telmesartan and any of these drugs used in combination with hydrochlorothiazide such as HYZAAR®) or other anti-hypertensive drugs such as LCZ 696, RXR agonists, glycogen synthase kinase-3 inhibitors, immune modulators, sympatholitics, beta-adrenergic blocking drugs (e.g., propranolol, atenolol, bisoprolol, carvedilol, metoprolol, or metoprolol tartate), alpha adrenergic blocking drugs (e.g., doxazocin, prazocin or alpha methyldopa) central alpha adrenergic agonists, peripheral vasodilators (e.g. hydralazine); beta-3 adrenergic receptor agonists, 11beta-HSD1 inhibitors, neutral endopeptidase inhibitors (e.g., thiorphan and phosphoramidon), aldosterone antagonists, aldosterone synthase inhibitors, renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924), N-morpholino derivatives (U.S. Pat. No. 5,055,466), N-heterocyclic alcohols (U.S. Pat. No. 4,885,292) and pyrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643), enalkrein, RO 42-5892, A 65317, CP 80794, ES 1005, ES 8891, SQ 34017, aliskiren (2(S),4(S),5(S),7(S)—N-(2-carbamoyl-2-methylpropyl)-5-amino-4-hydroxy-2,7-diisopropyl-8-[4-methoxy-3-(3-methoxypropoxy)-phenyl]-octanamid hemifumarate) SPP600, SPP630 and SPP635), endothelin receptor antagonists, phosphodiesterase-5 inhibitors (e.g. sildenafil, tadalfil and vardenafil), vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), lipid lowering agents e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), cerivastatin, and fluvastatin (particularly the sodium salt sold in LESCOL®); a cholesterol absorption inhibitor such as ezetimibe (ZETIA®) and ezetimibe in combination with any other lipid lowering agents such as the HMG-CoA reductase inhibitors noted above and particularly with simvastatin (VYTORIN®) or with atorvastatin calcium; HDL-raising drugs, (e.g., niacin and nicotinic acid receptor agonists, and extended- or controlled-release versions thereof, and/or with an HMG-CoA reductase inhibitor; niacin receptor agonists such as acipimox and acifran, as well as niacin receptor partial agonists; glucagon receptor antagonists (e.g., MK-3577, MK-0893, LY-2409021 and KT6-971); bile acid sequestering agents (e.g., colestilan, colestimide, colesevalam hydrochloride, colestipol, cholestyramine, and dialkylaminoalkyl derivatives of a cross-linked dextran), acyl CoA:cholesterol acyltransferase inhibitors, (e.g., avasimibe); agents intended for use in inflammatory conditions, such as aspirin, non-steroidal anti-inflammatory drugs or NSAIDs, glucocorticoids, and selective cyclooxygenase-2 or COX-2 inhibitors; glucokinase activators (GKAs) (e.g., AZD6370); inhibitors of 11β-hydroxysteroid dehydrogenase type 1, (e.g., such as those disclosed in U.S. Pat. No. 6,730,690, and LY-2523199); CETP inhibitors (e.g., anacetrapib, evacetrapib, and torcetrapib); inhibitors of fructose 1,6-bisphosphatase, (e.g., such as those disclosed in U.S. Pat. Nos. 6,054,587; 6,110,903; 6,284,748; 6,399,782; and 6,489,476); inhibitors of acetyl CoA carboxylase-1 or 2 (ACC1 or ACC2); PCSK9 inhibitors; GPR-40 partial agonists; SCD modulators; inhibitors of fatty acid synthase; amylin and amylin analogues (e.g., pramlintide); including pharmaceutically acceptable salt forms of the above active agents where chemically possible.

Furthermore, the present disclosure contemplates combination therapy with agents and methods for promoting weight loss, such as agents that stimulate metabolism or decrease appetite, and modified diets and/or exercise regimens to promote weight loss.

The compounds of the present disclosure may be used in combination with one or more other agent in any manner appropriate under the circumstances. In one embodiment, treatment with the at least one active agent and at least one compound of the present disclosure is maintained over a period of time. In another embodiment, treatment with the at least one active agent is reduced or discontinued (e.g., when the subject is stable), while treatment with the subject compound(s) of the present disclosure is maintained at a constant dosing regimen. In a further embodiment, treatment with the at least one active agent is reduced or discontinued (e.g., when the subject is stable), while treatment with the subject compound(s) of the present disclosure is reduced (e.g., lower dose, less frequent dosing or shorter treatment regimen). In yet another embodiment, treatment with the at least one active agent is reduced or discontinued (e.g., when the subject is stable), and treatment with the subject compound(s) of the present disclosure is increased (e.g., higher dose, more frequent dosing or longer treatment regimen). In yet another embodiment, treatment with the at least one active agent is maintained and treatment with the subject compound d(s) of the present disclosure is reduced or discontinued (e.g., lower dose, less frequent dosing or shorter treatment regimen). In yet another embodiment, treatment with the at least one active agent and treatment with the subject compound(s) of the present disclosure are reduced or discontinued (e.g., lower dose, less frequent dosing or shorter treatment regimen).

In certain instances, the combination provides an enhanced effect relative to either component alone; in some cases, the combination provides a supra-additive or synergistic effect relative to the combined or additive effects of the components. A variety of combinations of the subject compounds and the additional agent or therapy may be employed, used either sequentially or simultaneously. For multiple dosages, the two agents may directly alternate, or two or more doses of one agent may be alternated with a single dose of the other agent, for example. Simultaneous administration of both agents may also be alternated or otherwise interspersed with dosages of the individual agents. In some cases, the time between dosages may be for a period from about 1-6 hours, to about 6-12 hours, to about 12-24 hours, to about 1-2 days, to about 1-2 week or longer following the initiation of treatment.

Compositions

Aspects of the invention also include compositions, e.g., compositions including a subject compound (e.g., as described herein) formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release. In some embodiments, the subject compound is formulated for depot release.

In some embodiments of the present invention, a pharmaceutical composition is provided, comprising, or consisting essentially of, a compound of the present invention, or a pharmaceutically acceptable salt, isomer, tautomer or prodrug thereof, and further comprising one or more additional agent of interest (e.g., as described herein).

The subject compound and second agent, as well as any additional therapeutic agents for combination therapies, can be administered orally, subcutaneously, intramuscularly, intranasally, parenterally, or other route. The subject compound and second agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ. In certain cases, the therapeutic agents can be administered intranasally.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

Kits

Aspects of the invention further include kits for use in practicing the subject methods and compositions. The compounds of the invention can be included as reagents in kits for use in, for example, the methodologies described above.

A kit can include a compound (e.g., as described herein); and one or more components selected from the group consisting of an additional active agent, a buffer, a solvent, a standard and instructions for use.

The one or more components of the kit may be provided in separate containers (e.g., separate tubes, bottles, or wells in a multi-well strip or plate).

The compounds of the kits may be provided in a liquid composition, such as any suitable buffer. Alternatively, the compounds of the kits may be provided in a dry composition (e.g., may be lyophilized), and the kit may optionally include one or more buffers for reconstituting the dry compound. In certain aspects, the kit may include aliquots of the compound provided in separate containers (e.g., separate tubes, bottles, or wells in a multi-well strip or plate).

In addition, one or more components may be combined into a single container, e.g., a glass or plastic vial, tube or bottle. In certain instances, the kit may further include a container (e.g., such as a box, a bag, an insulated container, a bottle, tube, etc.) in which all of the components (and their separate containers) are present. The kit may further include packaging that is separate from or attached to the kit container and upon which is printed information about the kit, the components of the and/or instructions for use of the kit.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, DVD, portable flash drive, etc., on which the information has been recorded. Yet another means that may be present is a website address which may be used via the Internet to access the information at a removed site. Any convenient means may be present in the kits.

Utility

The compounds and methods of the invention, e.g., as described herein, find use in a variety of applications. Applications of interest include, but are not limited to: research applications and therapeutic applications. Methods of the invention find use in a variety of different applications including any convenient application where treatment of a metabolic syndrome-related disease, or symptom thereof, e.g., hyperlipidemia, type 2 diabetes, fatty liver disease, obesity, cardiovascular disease, stroke, etc., is desired.

The subject compounds and methods find use in a variety of research applications. The subject compounds and methods may be used in the optimization of the bioavailability and metabolic stability of compounds.

The subject compounds and methods find use in a variety of therapeutic applications. Therapeutic applications of interest include those applications in which metabolic disorder is the cause or a compounding factor in disease progression. As such, the subject compounds find use in the treatment of a variety of different conditions in which mitochondrial inhibition and/or treatment of metabolic syndrome-related disease in the host is desired. For example, the subject compounds may find use in treatment for obesity, insulin sensitivity, and diseases that derive from mitochondrial aging and loss of function. The subject compounds can find use as an alternative to calorie restriction.

As such, the subject compounds find use in the treatment of a variety of different conditions in which treatment of a metabolic syndrome-related disease in the host is desired (e.g., as described herein).

In additional embodiments, any of the compounds described herein may be administered to a patient for the treatment of cancer. For example, any of the compounds may be used to treat a cancer including but not limited to, e.g., Acute Lymphoblastic Leukemia (ALL), Acute Myeloid Leukemia (AML), Adrenocortical Carcinoma, AIDS-Related Cancers (e.g., Kaposi Sarcoma, Lymphoma, etc.), Anal Cancer, Appendix Cancer, Astrocytomas, Atypical Teratoid/Rhabdoid Tumor, Basal Cell Carcinoma, Bile Duct Cancer (Extrahepatic), Bladder Cancer, Bone Cancer (e.g., Ewing Sarcoma, Osteosarcoma and Malignant Fibrous Histiocytoma, etc.), Brain Stem Glioma, Brain Tumors (e.g., Astrocytomas, Central Nervous System Embryonal Tumors, Central Nervous System Germ Cell Tumors, Craniopharyngioma, Ependymoma, etc.), Breast Cancer (e.g., female breast cancer, male breast cancer, childhood breast cancer, etc.), Bronchial Tumors, Burkitt Lymphoma, Carcinoid Tumor (e.g., Childhood, Gastrointestinal, etc.), Carcinoma of Unknown Primary, Cardiac (Heart) Tumors, Central Nervous System (e.g., Atypical Teratoid/Rhabdoid Tumor, Embryonal Tumors, Germ Cell Tumor, Lymphoma, etc.), Cervical Cancer, Childhood Cancers, Chordoma, Chronic Lymphocytic Leukemia (CLL), Chronic Myelogenous Leukemia (CML), Chronic Myeloproliferative Neoplasms, Colon Cancer, Colorectal Cancer, Craniopharyngioma, Cutaneous T-Cell Lymphoma, Duct (e.g., Bile Duct, Extrahepatic, etc.), Ductal Carcinoma In Situ (DCIS), Embryonal Tumors, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer (e.g., Intraocular Melanoma, Retinoblastoma, etc.), Fibrous Histiocytoma of Bone (e.g., Malignant, Osteosarcoma, etc.), Gallbladder Cancer, Gastric (Stomach) Cancer, Gastrointestinal Carcinoid Tumor, Gastrointestinal Stromal Tumors (GIST), Germ Cell Tumor (e.g., Extracranial, Extragonadal, Ovarian, Testicular, etc.), Gestational Trophoblastic Disease, Glioma, Hairy Cell Leukemia, Head and Neck Cancer, Heart Cancer, Hepatocellular (Liver) Cancer, Histiocytosis (e.g., Langerhans Cell, etc.), Hodgkin Lymphoma, Hypopharyngeal Cancer, Intraocular Melanoma, Islet Cell Tumors (e.g., Pancreatic Neuroendocrine Tumors, etc.), Kaposi Sarcoma, Kidney Cancer (e.g., Renal Cell, Wilms Tumor, Childhood Kidney Tumors, etc.), Langerhans Cell Histiocytosis, Laryngeal Cancer, Leukemia (e.g., Acute Lymphoblastic (ALL), Acute Myeloid (AML), Chronic Lymphocytic (CLL), Chronic Myelogenous (CML), Hairy Cell, etc.), Lip and Oral Cavity Cancer, Liver Cancer (Primary), Lobular Carcinoma In Situ (LCIS), Lung Cancer (e.g., Non-Small Cell, Small Cell, etc.), Lymphoma (e.g., AIDS-Related, Burkitt, Cutaneous T-Cell, Hodgkin, Non-Hodgkin, Primary Central Nervous System (CNS), etc.), Macroglobulinemia (e.g., Waldenström, etc.), Male Breast Cancer, Malignant Fibrous Histiocytoma of Bone and Osteosarcoma, Melanoma, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia (e.g., Chronic (CML), etc.), Myeloid Leukemia (e.g., Acute (AML), etc.), Myeloproliferative Neoplasms (e.g., Chronic, etc.), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer (e.g., Lip, etc.), Oropharyngeal Cancer, Osteosarcoma and Malignant Fibrous Histiocytoma of Bone, Ovarian Cancer (e.g., Epithelial, Germ Cell Tumor, Low Malignant Potential Tumor, etc.), Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Pleuropulmonary Blastoma, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (e.g., Ewing, Kaposi, Osteosarcoma, Rhabdomyosarcoma, Soft Tissue, Uterine, etc.), Sezary Syndrome, Skin Cancer (e.g., Childhood, Melanoma, Merkel Cell Carcinoma, Nonmelanoma, etc.), Small Cell Lung Cancer, Small Intestine Cancer, Soft Tissue Sarcoma, Squamous Cell Carcinoma, Squamous Neck Cancer (e.g., with Occult Primary, Metastatic, etc.), Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Ureter and Renal Pelvis Cancer, Urethral Cancer, Uterine Cancer (e.g., Endometrial, etc.), Uterine Sarcoma, Vaginal Cancer, Vulvar Cancer, Waldenström Macroglobulinemia, Wilms Tumor, and the like.

As would be understood, the method may involve administering a compound to a cancer patient.

The following example(s) is/are offered by way of illustration and not by way of limitation.

### Additional Embodiments

Additional embodiments are set forth in the following clauses.

Clause 1. A compound of formula (I):

HG-L-X (I)

wherein:

HG is headgroup selected from a heterocyclic group, a heteroaryl group, and a guanidine, wherein the head group is optionally substituted;

L is a linker; and

X is a charged group,

Provided that the compound is not:

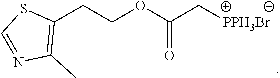

Clause 2. The compound of clause 1, wherein the headgroup is selected from a thiazole, a pyrazole, a thiophene, an oxazole, an oxadiazole, a tetrazole, a triazole, a pyridine, a pyrimidine, a pyrazine, a pyrazine, a triazine, a pyran, an oxazine, a thiazine a morpholine, a thiomorpholine, a piperidine and a piperazine.
Clause 3. The compound of clause 1 or 2, wherein the headgroup is selected from a thiazole, an oxadiazole, a tetrazole, a triazine, and a guanidine.
Clause 4. The compound of any one of clauses 1 to 3, wherein the headgroup is any one of formula (HG1)-(HG9):

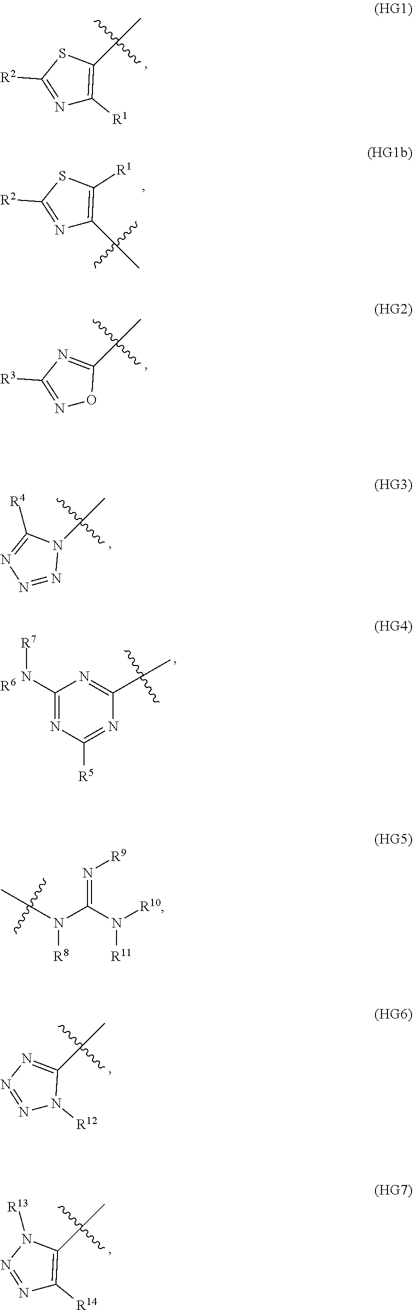

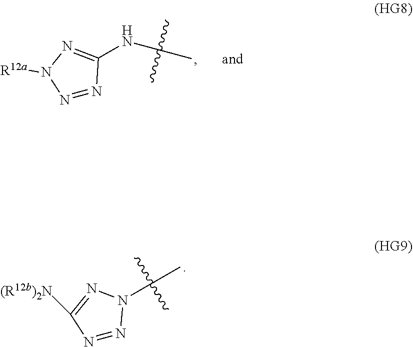

wherein:

R¹-R¹⁴ are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

Clause 5. The compound of clause 4, wherein the headgroup is of any one of formula (HG1a)-(HG9a):

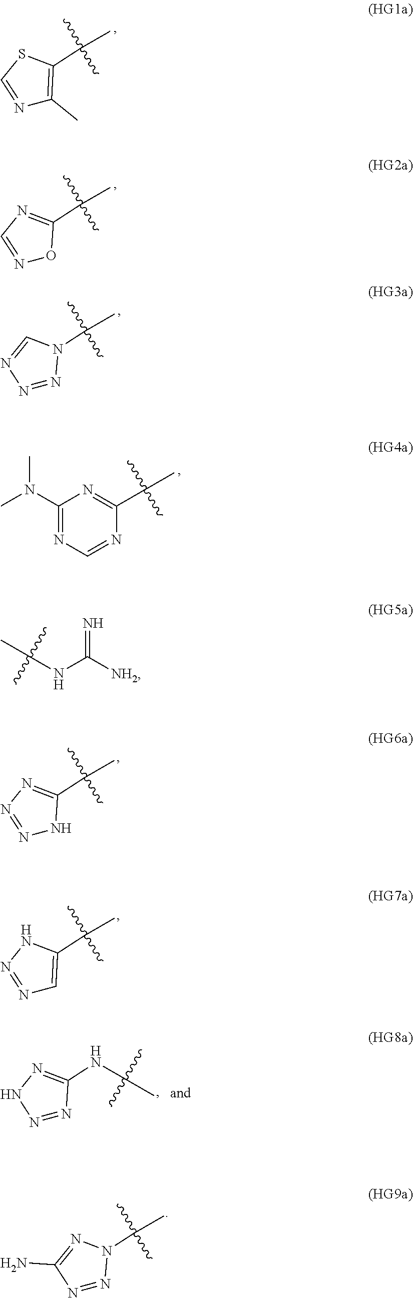

Clause 6. The compound of any one of clauses 1 to 5, wherein the linker is described by the formula (L1):

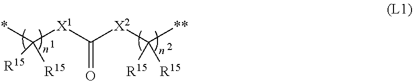

wherein:

* represents the point of connection to HG;

** represents the point of connection to X;

X¹ and X² are each independently selected from C(R¹⁵)₂, C(R¹⁵)₂(OCH₂CH₂O)ₙ₃, O, S and NR¹⁶;

each R¹⁵ is independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino and hydroxyl;

n¹ an integer from 0 to 10;

n² is an integer from 0 to 10; and

n³ is an integer from 1 to 20.

Clause 7. The compound of clause 6, wherein X¹ is selected from O, NH and S; and X² is C(R¹⁵)₂.
Clause 8. The compound of clause 6, wherein the linker is described by a structure selected from any one of (L2)-(L5):

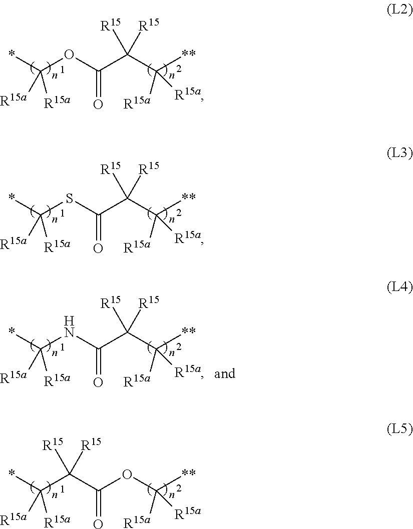

wherein:

* represents the point of connection to HG;

** represents the point of connection to X;

each R¹⁵ and R^{15a} are independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

n¹ an integer from 0 to 10;

n² is an integer from 0 to 10; and

n³ is an integer from 1 to 20.

Clause 9. The compound of clause 6, wherein the linker is described by a structure selected any one of (B1)-(B11):

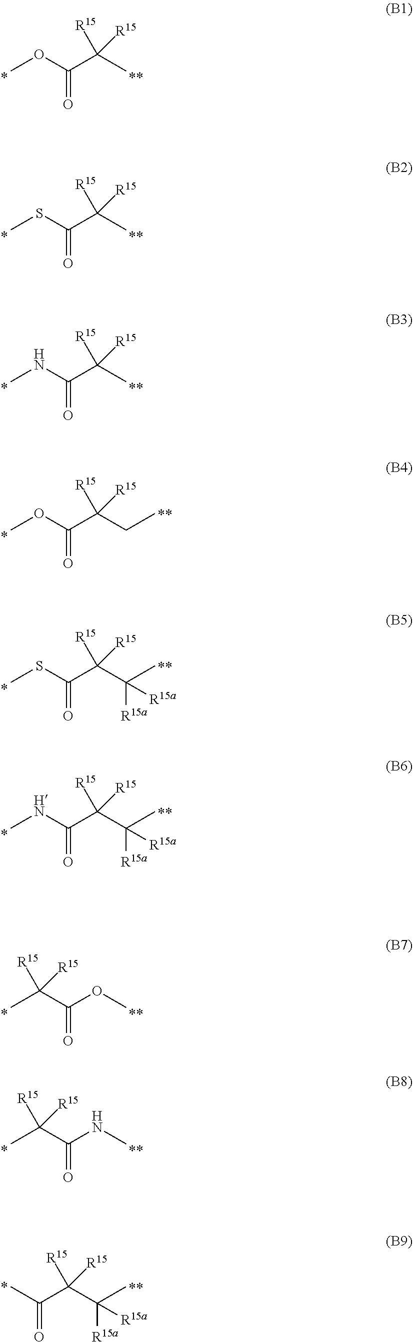

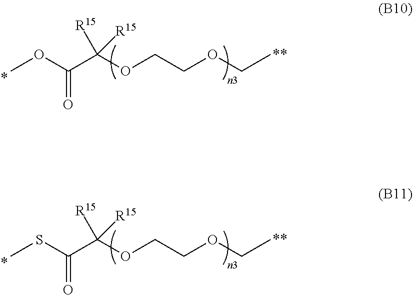

wherein:

* represents the point of connection to HG;

** represents the point of connection to X;

R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

n¹ an integer from 0 to 10;

n² is an integer from 0 to 10; and

n³ is an integer from 1 to 20.

Clause 10. The compound of any one of clauses 1 to 9 of the formula (IA) or (IB):

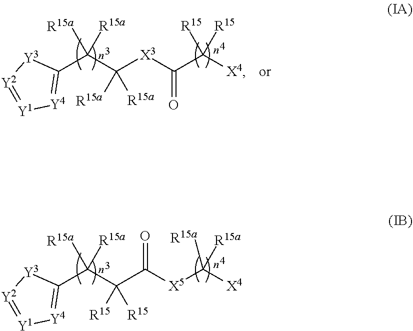

wherein:

Y¹, Y² and Y⁴ are each independently selected from N and CR¹⁵; Y³ is selected from S, O, NR¹⁶, and C(R¹⁵)₂;

X³ and X⁵ are each independently selected from C(R¹⁵)₂, O, S and NR¹⁶;

each R¹⁵ and R^{15a} are independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;

each R¹⁶ is independently selected from hydrogen, alkyl, substituted alkyl, aryl, substituted aryl, carboxyl, acyl, substituted acyl, amino, substituted amino, and hydroxyl;

X⁴ is a charged group;

n³ an integer from 0 to 10; and

n⁴ is an integer from 1 to 10.

Clause 11. The compound of clause 10 of the formula (IC) or (ID):

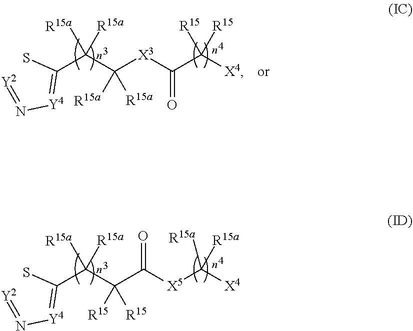

wherein:

Y² and Y⁴ are each CR¹⁵;

X³ and X⁵ are each independently selected from CR¹⁵, O, S and NR¹⁶;

each R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino, and hydroxyl;

X⁴ is a charged group;

n³ an integer from 0 to 10; and

n⁴ is an integer from 1 to 10.

Clause 12. The compound of clause 11 of the formula (IE):

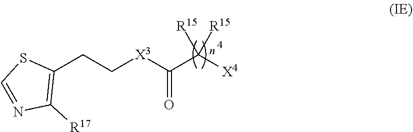

wherein:

X³ is selected from C(R¹⁵)₂, O, S and NR¹⁶;

each R¹⁵, and R¹⁷ are independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino, and hydroxyl;

X⁴ is a charged group; and

n⁴ is an integer from 1 to 10.

Clause 13. The compound of any one of clauses 1 to 12, wherein the charged group is selected from a phosphonium cation, an ammonium cation, a quaternary ammonium cation, a pyridinium cation, an imidazolium cation, a guanidine moiety and an arginine moiety.
Clause 14. The compound of clause 13, wherein the phosphonium cation is a triphenylphosphonium cation.
Clause 15. The compound of clause 13, wherein the quaternary ammonium cation is a triethylammonium ion.
Clause 16. The compound of any one of clauses 1 to 15, wherein the charged group is any one of formula (X1)-(X9):

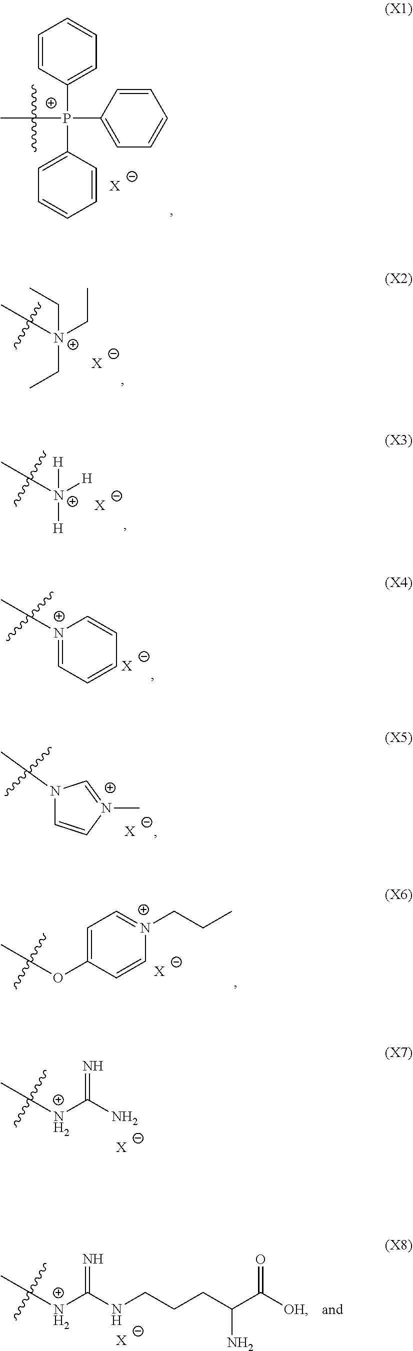

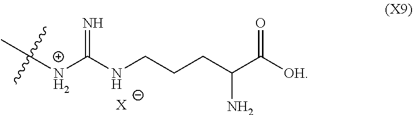

Clause 17. The compound of any one of clauses 1 to 16, wherein the charged group comprises a halide counterion.
Clause 18. The compound of clause 17, wherein the halide counterion is bromide.
Clause 19. The compound of any one of clauses 1 to 18, described by a structure in any one of Table 1 to Table 8.
Clause 20. A method of treating a subject having a metabolic syndrome-related disease or a symptom thereof, the method comprising:
administering to the subject a therapeutically effective amount of a compound of the formula:

HG-L-X (I)

wherein:

HG is headgroup selected from a heterocyclic group, a heteroaryl group, and a guanidine group, wherein the head group is optionally substituted;

L is a linker; and

X is a charged group.

Clause 21. The method of clause 20, wherein the disease is selected from hyperlipidemia, type 2 diabetes, fatty liver disease, obesity, cardiovascular disease and stroke.
Clause 22. The method of clause 20 or 21, wherein the symptom is selected from abdominal obesity, insulin resistance, hyperinsulinemia, high levels of blood fats, increased blood pressure, and elevated serum lipids.
Clause 23. The method of any one of clauses 20 to 22, wherein the headgroup is selected from a thiazole, a pyrazole, a thiophene, an oxazole, an oxadiazole, a tetrazole, a triazole, a pyridine, a pyrimidine, a pyrazine, a pyrazine, a triazine, a pyran, an oxazine, a thiazine a morpholine, a thiomorpholine, a piperidine and a piperazine.
Clause 24. The method of clause 23, wherein the headgroup is selected from a thiazole, an oxadiazole, a tetrazole, a triazine and a guanidine.
Clause 25. The method of any one of clauses 20 to 24, wherein the headgroup is any one of formula (HG1)-(HG9):

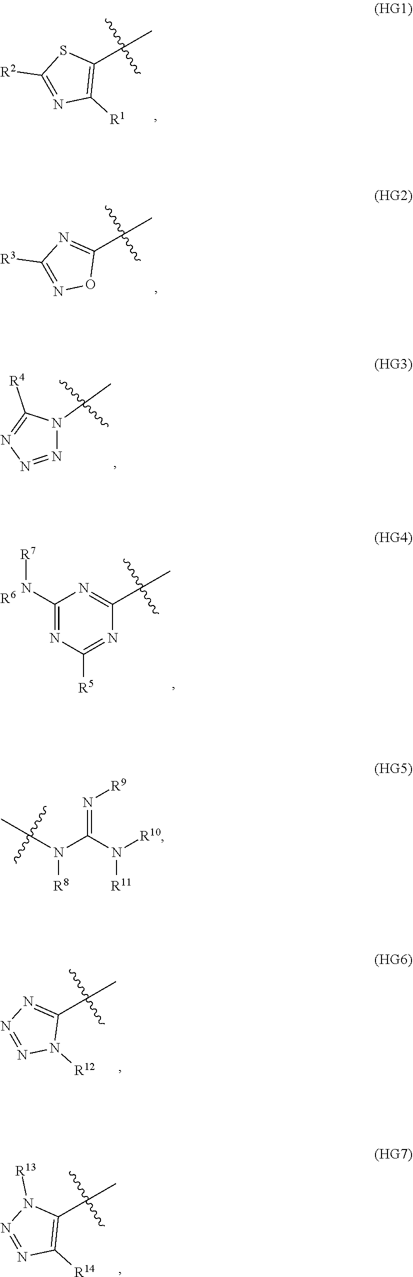

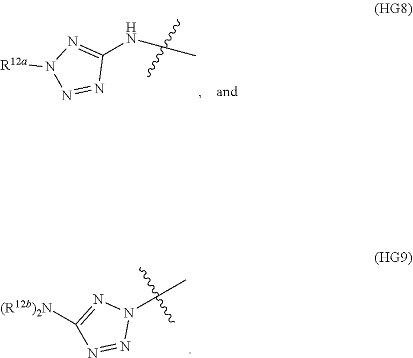

wherein:

R¹-R¹⁴ are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

Clause 26. The method of clause 25, wherein the headgroup is of any one of formula (HG1a)-(HG9a):

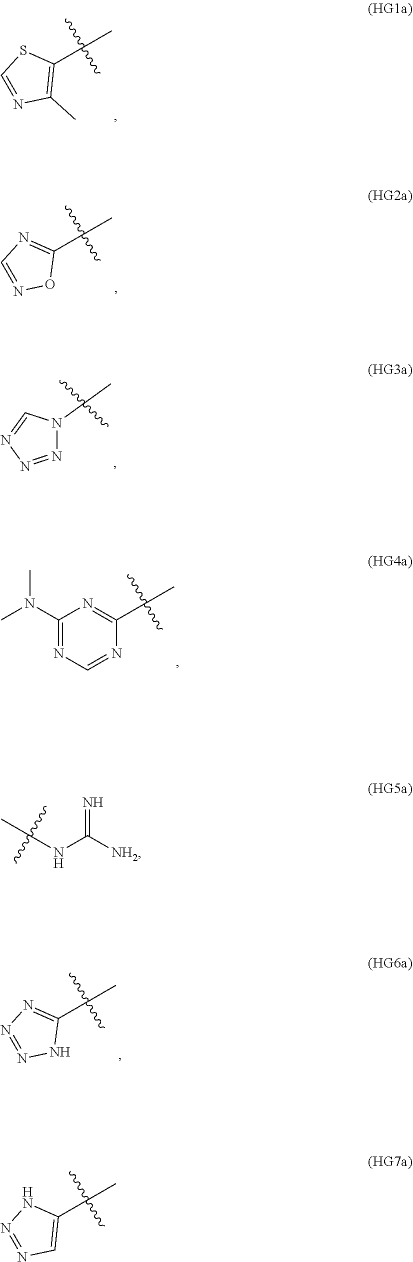

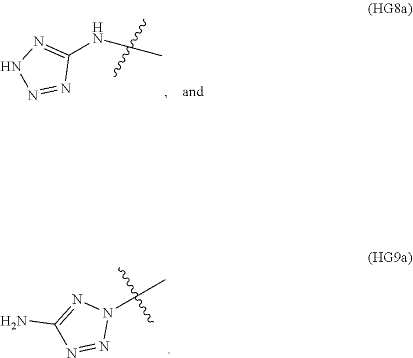 [SMILES: *Nc1nn[nH]n1 ; *n1nnc(N)n1]

Clause 27. The method of any one of clauses 20 to 26, wherein the linker is described by the formula (L1):

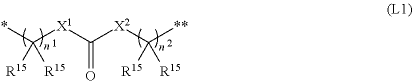

* represents the point of connection to HG;

** represents the point of connection to X;

X¹ and X² are each independently selected from C(R¹⁵)₂, C(R¹⁵)₂(OCH₂CH₂O)ₙ₃, O, S and NR¹⁶;

each R¹⁵ is independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino and hydroxyl;

n¹ an integer from 0 to 10;

n² is an integer from 0 to 10; and

n³ is an integer from 1 to 20.

Clause 28. The method of clause 27, wherein the linker is described by a structure selected from any one of (L2)-(L5):

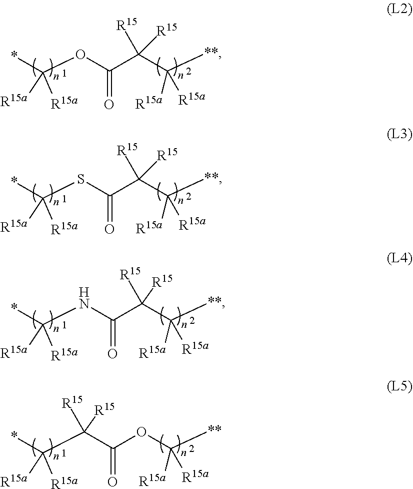

wherein:

* represents the point of connection to HG;

** represents the point of connection to X;

each R¹⁵ and R^{15a} are independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

n¹ an integer from 0 to 10;

n² is an integer from 0 to 10; and

n³ is an integer from 1 to 20.

Clause 29. The method of clause 27, wherein the linker is described by a structure selected from any one of (B1)-(B11):

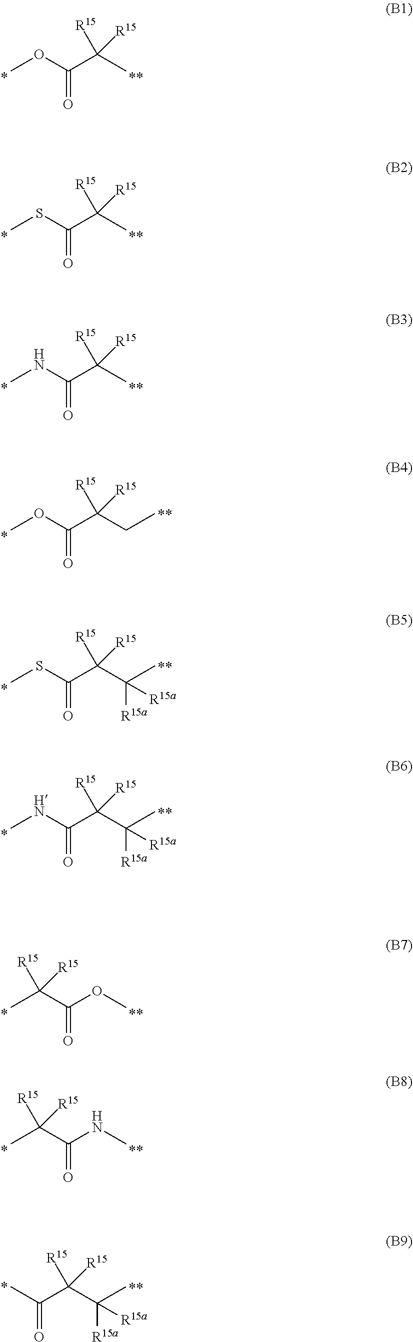

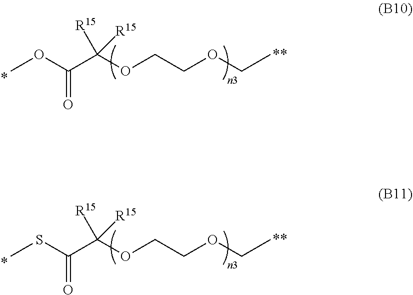

wherein:

* represents the point of connection to HG;

** represents the point of connection to X;

R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

n¹ an integer from 0 to 10;

n² is an integer from 0 to 10; and

n³ is an integer from 1 to 20.

Clause 30. The method of any one of clauses 20 to 29, wherein the compound is of the formula (IA) or (IB):

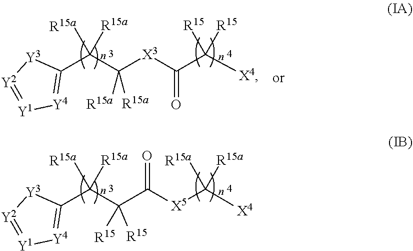

wherein:

Y¹, Y² and Y⁴ are each independently selected from N and CR¹⁵; Y³ is selected from S, O, NR¹⁶, and C(R¹⁵)₂;

X³ and X⁵ are each independently selected from C(R¹⁵)₂, O, S and NR¹⁶;

each R¹⁵ and R^{15a} are independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;

each R¹⁶ is independently selected from hydrogen, alkyl, substituted alkyl, aryl, substituted aryl, carboxyl, acyl, substituted acyl, amino, substituted amino, and hydroxyl;

X⁴ is a charged group;

n³ an integer from 0 to 10; and

n⁴ is an integer from 1 to 10.

Clause 31. The method of clause 30, wherein the compound is of the formula (IC) or (ID):

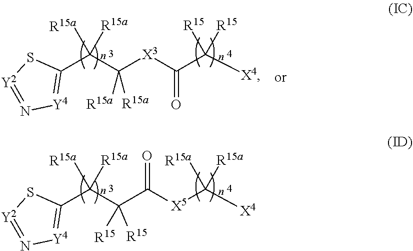

wherein:

Y² and Y⁴ are each CR¹⁵;

X³ and X⁵ are each independently selected from C(R¹⁵)₂, O, S and NR¹⁶;

each R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino, and hydroxyl;

X⁴ is a charged group;

n³ an integer from 0 to 10; and

n⁴ is an integer from 1 to 10.

Clause 32. The method of clause 31, wherein the compound is of the formula (IE):

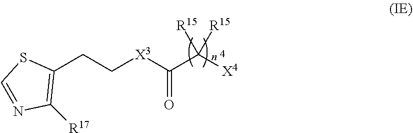

wherein:

X³ is selected from C(R¹⁵)₂, O, S and NR¹⁶;

each R¹⁵, and R¹⁷ are independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;

R¹⁶ is selected from hydrogen, alkyl, substituted alkyl, aryl, substituted aryl, carboxyl, acyl, substituted acyl, amino, substituted amino, and hydroxyl;

X⁴ is a charged group; and

n⁴ is an integer from 1 to 10.

Clause 33. The method of any one of clauses 20 to 32, wherein the charged group is selected from a phosphonium cation, an ammonium cation, a quaternary ammonium cation, a pyridinium cation, an imidazolium cation, a guanidine moiety, and an arginine moiety.
Clause 34. The method of clause 33, wherein the phosphonium cation is a triphenylphosphonium cation.
Clause 35. The method of clause 33, wherein the quaternary ammonium cation is a triethylammonium ion.
Clause 36. The method of any one of clauses 20 to 35, wherein the charged group is any one of formula (X1)-(X7):

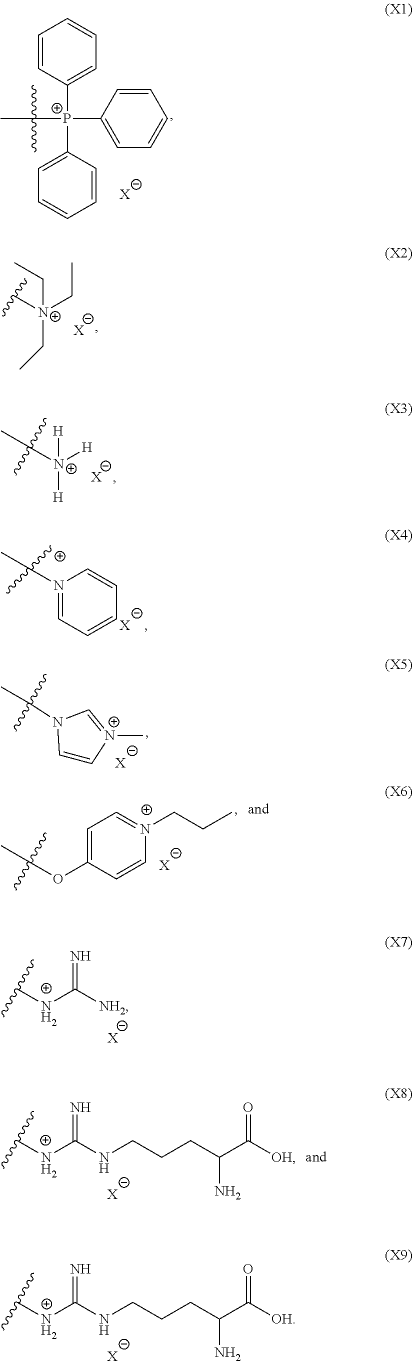

Clause 37. The method of any one of clauses 20 to 36, wherein the charged group comprises a halide counterion.
Clause 38. The method of clause 37, wherein the halide counterion is bromide.
Clause 39. The method of any one of clauses 20 to 38, described by a structure in any one of Table 1 to Table 8.
Clause 40. A method of treating cancer comprising administering any of the compounds of clauses 1-19 to a cancer patient.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Synthesis of Exemplary Compounds

Compounds may be prepared using any convenient method. Many general references providing commonly known chemical synthetic schemes and conditions useful for synthesizing the disclosed compounds are also available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978). Reactions may be monitored by thin layer chromatography (TLC), LC/MS and reaction products characterized by LC/MS and ¹H NMR. Intermediates and final products are purified by silica gel chromatography or by reverse phase HPLC.

For example, exemplary compounds may be prepared by similar methods to those described by Barile et al. “Inhibiting platelet-stimulated blood coagulation by inhibition of mitochondrial respiration.” Proc Natl Acad Sci U.S.A., (2012), 109(7): 2539-2543.

Exemplary synthetic scheme 1, which can be adapted for the synthesis of subject compounds, is shown below:

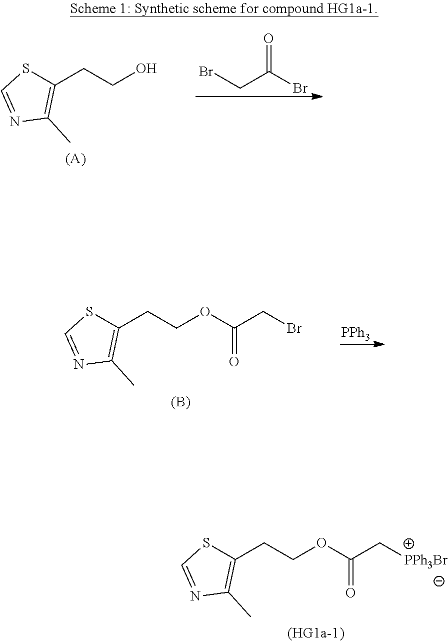

Preparation of 2-(4-Methylthiazol-5-yl)Ethyl 2-Bromoacetate (B). Under an atmosphere of N₂, 2-(4-methylthiazol-5-yl)ethanol (A) (970 mg) was dissolved in dry chloroform (4 mL). A total of 1.4 g of 2-bromoacetyl bromide was added drop-wise over the course of 30 min at 0° C. The reaction mixture was then stirred at room temperature for 2 h before saturated NaHCO₃ (20 mL) was added. The mixture was then extracted with chloroform (3×20 mL), the combined organic layers were dried with anhydrous MgSO₄, and the solvent was removed under reduced pressure. The crude product was purified using column chromatography with chloroform as the eluent to give the final product (850 mg, 48% yield): ¹H NMR (400 MHz, CDCl₃) δ 8.56 (s, 1H), 4.29 (t, 2H, 3 J=6.5 Hz), 3.80 (s, 2H), 3.10 (t, 2H, 3 J=6.5 Hz), 2.38 (s, 3H).

Preparation of (2-(2-(4-Methylthiazol-5-yl)Ethoxy)-2-Oxoethyl) Triphenylphosphonium Bromide (HG1a-1). Under an atmosphere of N₂, 2-(4-methylthiazol-5-yl)ethyl 2-bromoacetate (176 mg) and triphenylphosphine (175 mg) were dissolved in toluene (1.5 mL). The reaction mixture was stirred for 48 h at room temperature. The resulting white precipitate was filtered, triturated with toluene, and purified by recrystallization from ethanol to give the final product (110 mg, 31% yield): ¹H NMR (400 MHz, D₂O) δ 8.63 (s, 1H), 7.60-7.85 (m, 15H), 5.65 (d, 2H, 3 J=13.6 Hz), 4.22 (t, 2H, 3 J=5.7 Hz), 2.93 (t, 2H, 3 J=5.7 Hz), 2.13 (s, 3H).

The following exemplary compounds were prepared by using and adapting the synthetic procedures shown above:

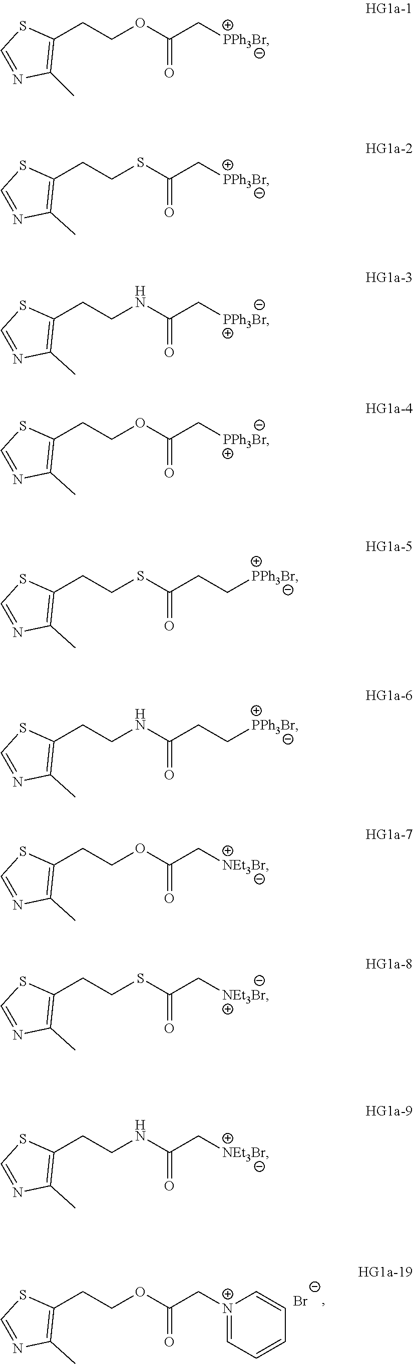

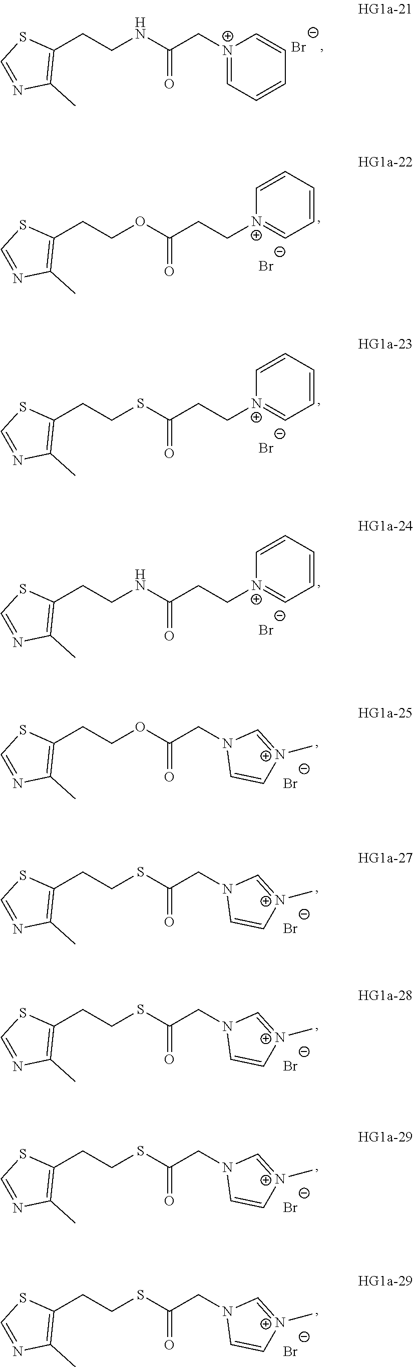

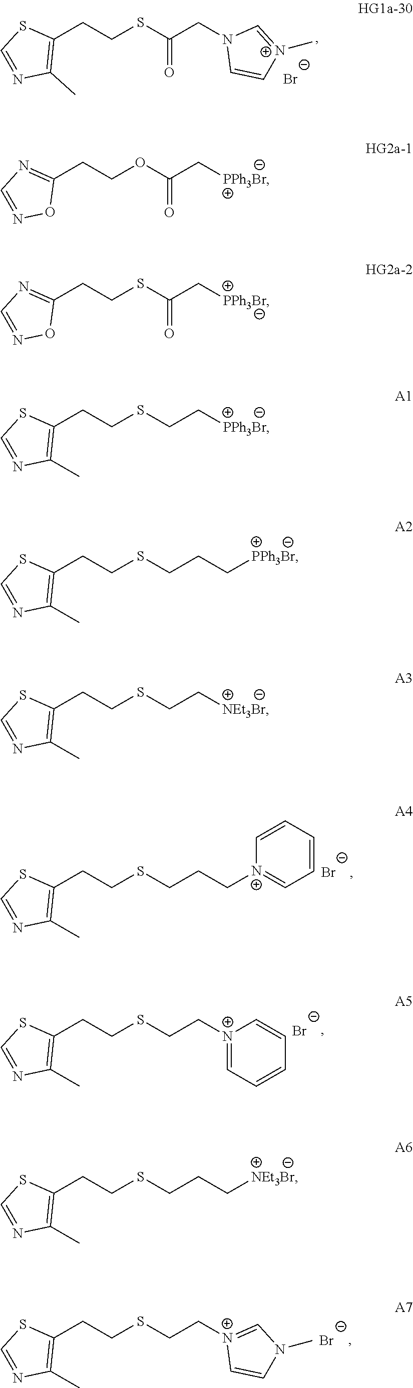

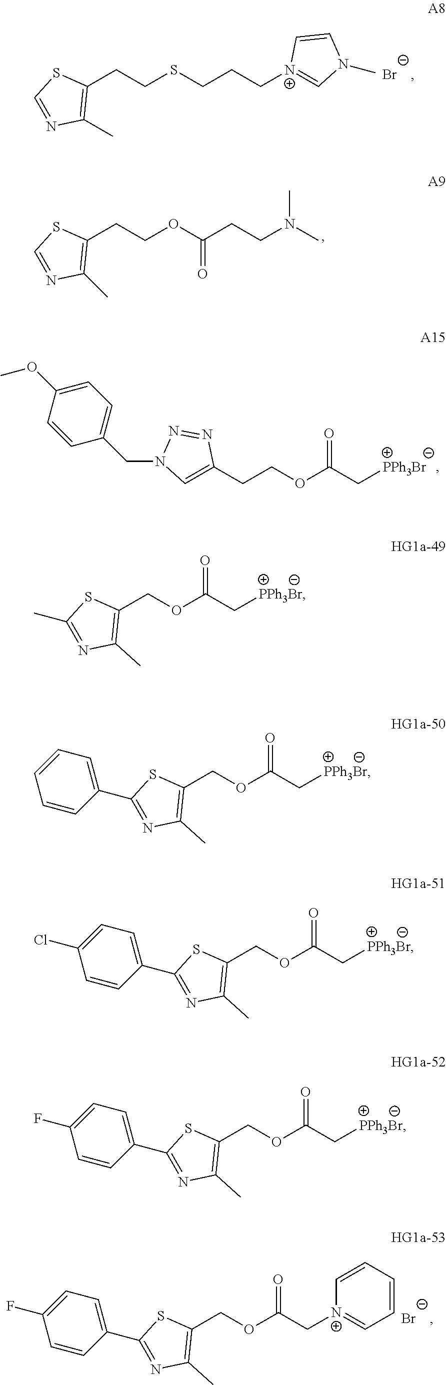

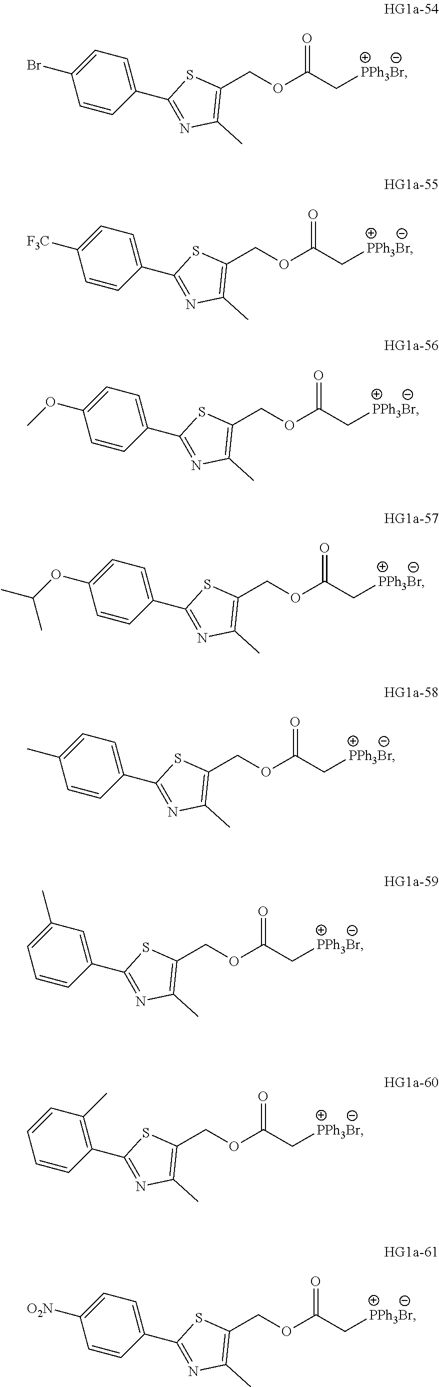

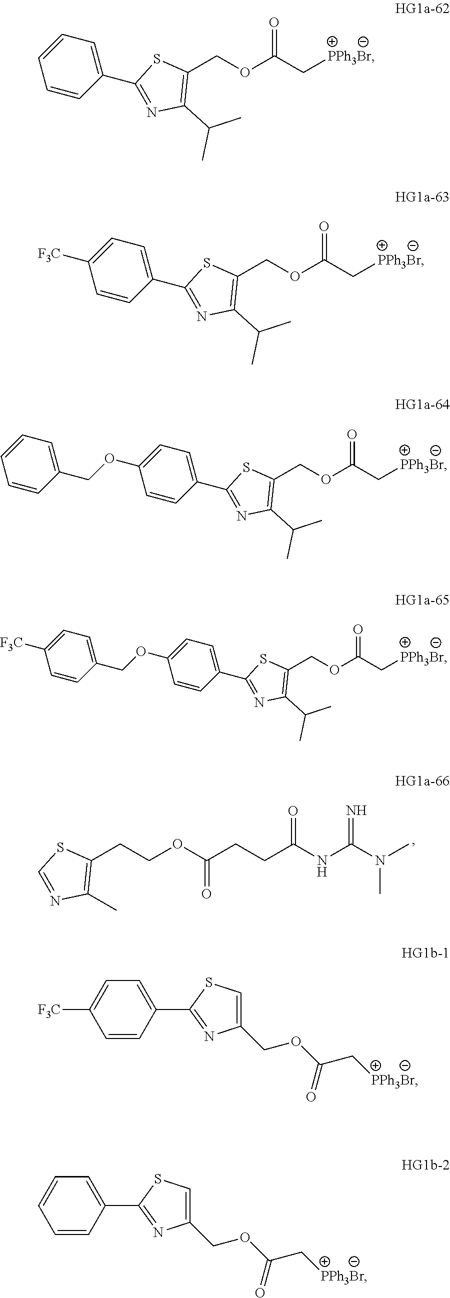

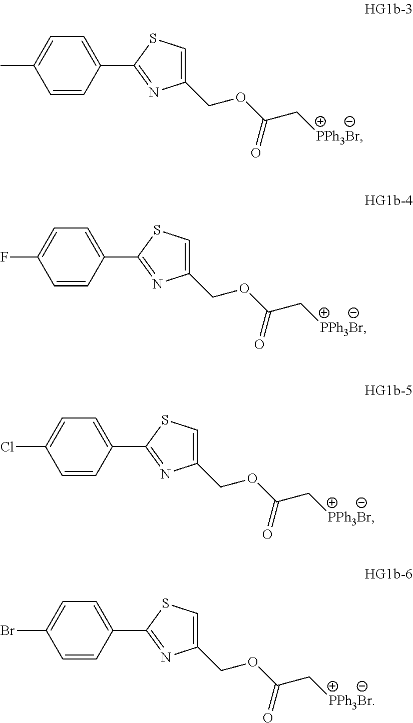

### Example 2: Biological Assays of Exemplary Compound HG1a-1

Introduction

Ageing is associated with decline in mitochondrial operation and the accumulation of abnormal mitochondria (Lopez-Otin et al., Cell (2013), 153, pp. 1194-1217) lead to metabolic disorders (Kumarjha et al., Biochimica et Biophysica Act (BBA)—Molecular Basis of Disease., (2017), 1863:5, pp. 1132-1146).

Mitochondria are organized inside cells to form an interconnected and dynamic network, regulated by mitochondrial dynamics. Alteration of mitochondrial dynamics in ageing could explain the accumulation of mitochondrial damage and be viewed as a mechanism linking a loss of mitochondrial fitness with a causative role in the pathogenesis of metabolic syndrome of ageing (Sebastian et al., Trends in Molecular Medicine. (2017), 23:3, p. 201-215).

Hindering the process of mitochondrial decay (mito-decay) and along with it, the course of ageing and metabolic syndrome has become a baffling conundrum for scientists.

Metabolic syndrome is a multi-systemic deterioration and consists mainly of insulin resistance, obesity/abdominal obesity, increased inflammatory peptides and risks for many age-related diseases (Rudin et al., Immunity & Ageing (2005), 2:1). Hence, increased adipose tissue is not simply a reservoir for excess nutrients, but rather an active and dynamic organ capable of expressing harmful factors and inflammatory agents which accelerate metabolic syndrome of ageing including obesity, type 2 diabetes mellitus (T2DM) and heart diseases.

Therefore, curing obesity, diabetes and pre-diabetic irregularities are the priorities to promote healthy ageing and metabolic syndrome alleviation. Although, calorie restriction and exercise are the first line of treatment, pharmacological treatment for spontaneous T2DM and obesity can be an effective method too, for example metformin. Metformin is a leading anti-diabetic drug which mimics the beneficial effects of calorie restriction by activating AMP-activated kinase (AMPK); the best documented method of slowing and reversing biomarkers of human ageing including obesity and insulin resistance (Choi et al., Mol. Cells., (2013), 36:4, pp. 279-287).

Having this perception and focusing on colossal impact of mitochondria quality on ageing, herein, we investigate the theory of health spanning by flexible inhibition of mitochondrial CcO complex IV. This application could better the mito-dynamics and thwart the metabolic syndrome of ageing. Mammalian CcO is the terminal complex (complex IV) of the electron transfer chain leads to ATP synthesis. Discoveries postulate that ageing occurs in process of mitochondria depreciation and decay during their constant operation of providing energy, then causes deregulated mito-dynamics, damaged DNA and escalated free radicals (ROS).

In this regard, we scrutinized the effects of the mitochondria operation setback on mitochondria fitness and healthy ageing. Moderate mitochondrial inhibition in mice could rectify the mitochondria quality, suppress age-related body fat mass storage specifically in visceral depot, enhance energy metabolism, promote physical activity, reduce free radicals generation, boost mitochondria biogenesis, increase metabolic shift to glycolysis, better the insulin sensitivity and glucose uptake by activating AMPK and keep healthy plasma lipid profile. Reflecting well accepted potential of flexible mitochondrial inhibition, herein we provide complementary dossier on age-linked mito-decay and metabolic syndrome of ageing.

Also disclosed herein is a new model for a calorie restriction (CR) mimetic profile. Moderate inhibition of mitochondrial cytochrome c oxidase complex IV in mice can resemble energy restriction virtues extensively. Cytochrome c oxidase is the component of the mitochondrial respiratory chain that catalyzes the reduction of oxygen to water to produce ATP. This approach can lead to improved mitochondria integrity and fitness through a balanced respiration maintaining lower ATP production associated with low ROS formation, reduced inflammatory markers and upgraded homeostatic metabolism. To evaluate this hypothesis, in certain experiments discussed herein, we utilized male C57BL/6 mice divided into three groups: a calorie restriction model group, an ad libitum (AL)-fed control group and a treated group (e.g., treated with a subject compound that can modulate mitochondrial activity for a period of 18-20 months consecutively). The results discussed herein indicate a major biocellular change in which a greater dynamic of more efficient mitochondria is demonstrated, highly decreased pro inflammatory, cellular damaging factors and metabolic complications associated with ageing. Accordingly, treatment with a subject compound can be a convenient alternative to CR.

Mitochondrial Fitness and Bioenergetic Efficiency

Previously, the inventors developed series of derivatives of the tetrazole, thiazole, and 1,2,3-triazole families that were thought to be reversible inhibitors of the cytochrome c oxidase (CcO) model and characterized as possible moderate inhibitors of mitochondrial respiration (Barile et al. (2012), Proceedings of the National Academy of Sciences of the United States of America, 109:7, pp. 2539-2543). In this research, a triphenyl phosphonium thiazole derivative (referred to herein as HG1a-1) was randomly selected. Overall, no mice died due to oral administration of compound during 18-20 months. To investigate long-term inhibitor administration, a lower dose was tested in drinking water, which could potentially be translatable to humans. We observed that 95%-99% of an exemplary subject compound was maintained intact in drinking water (pH=7-7.4) at room temperature for 20-30 days. Also, dramatic body weight changes were not observed and all the transformations were gradual and based on the compound mechanism of action on metabolism.

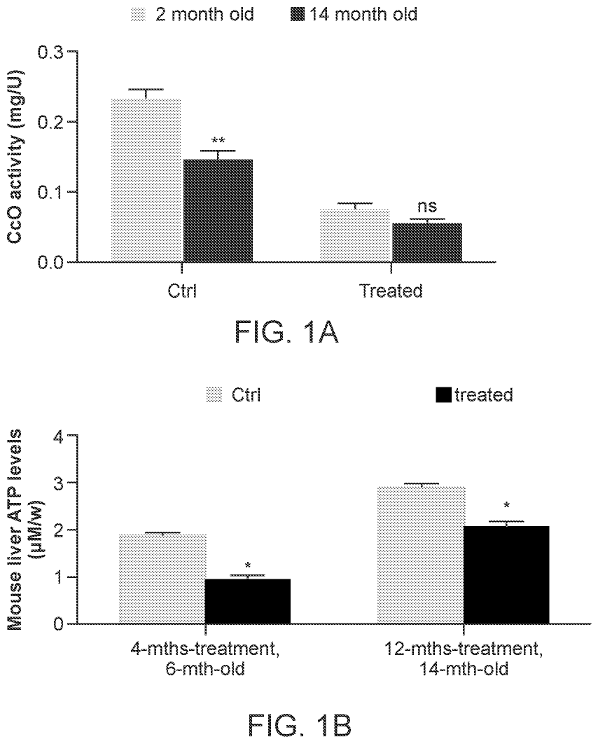

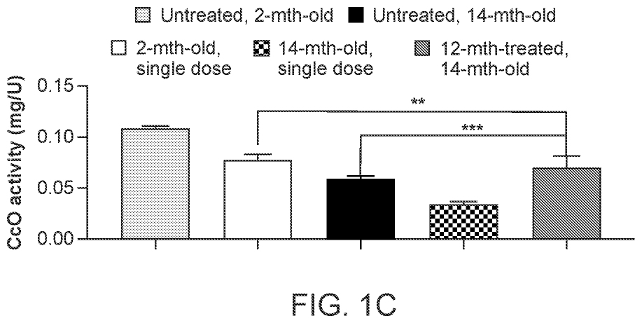

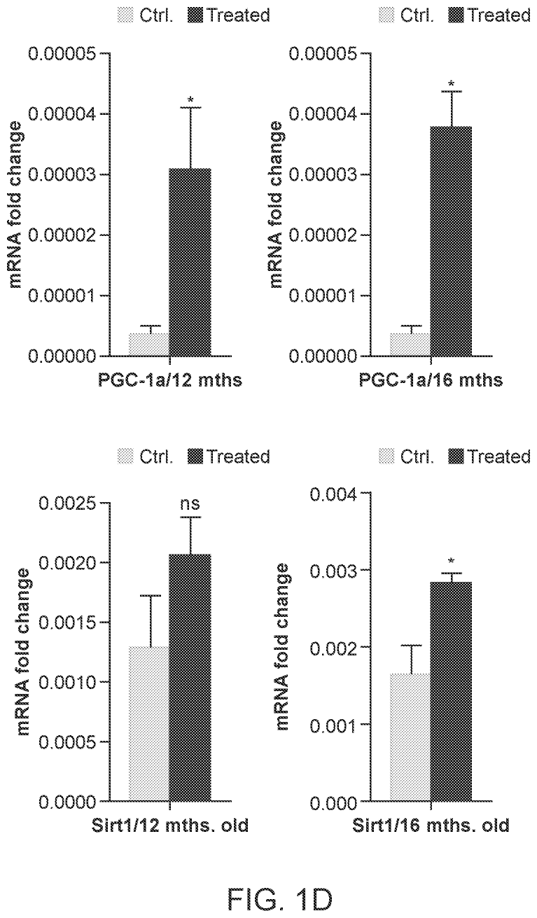

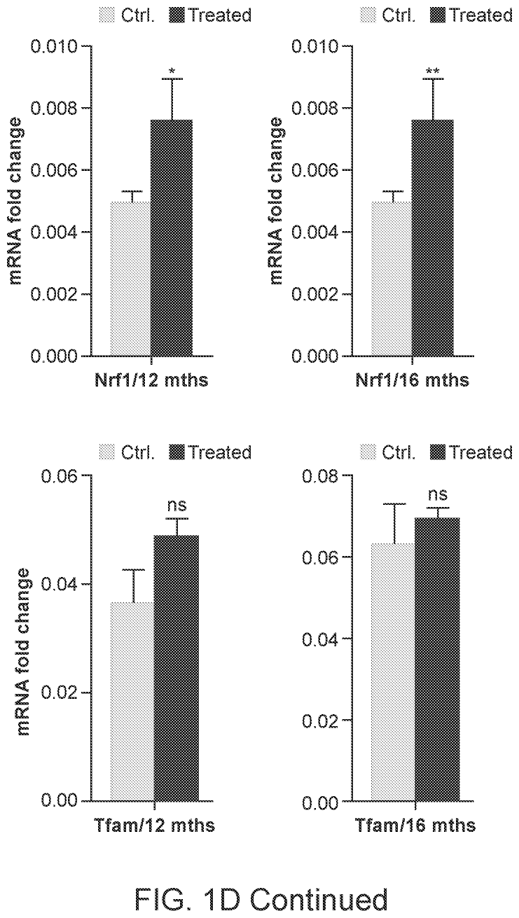

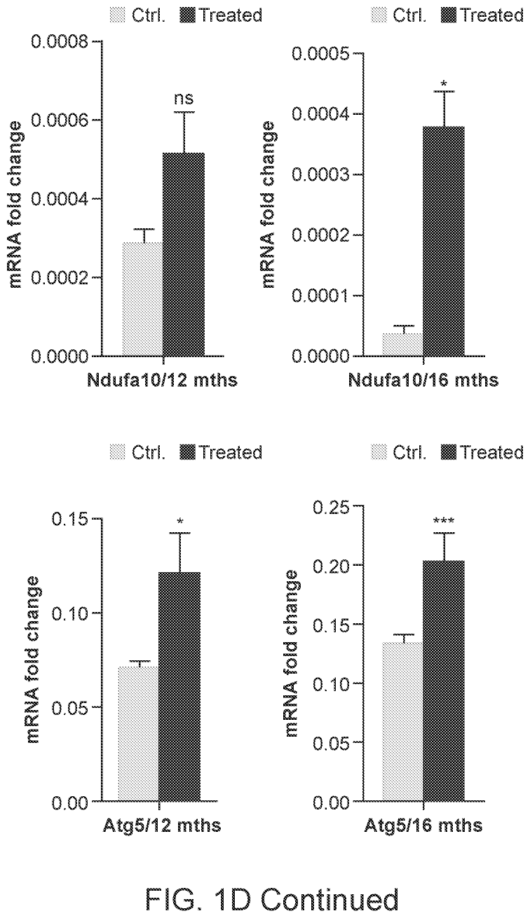

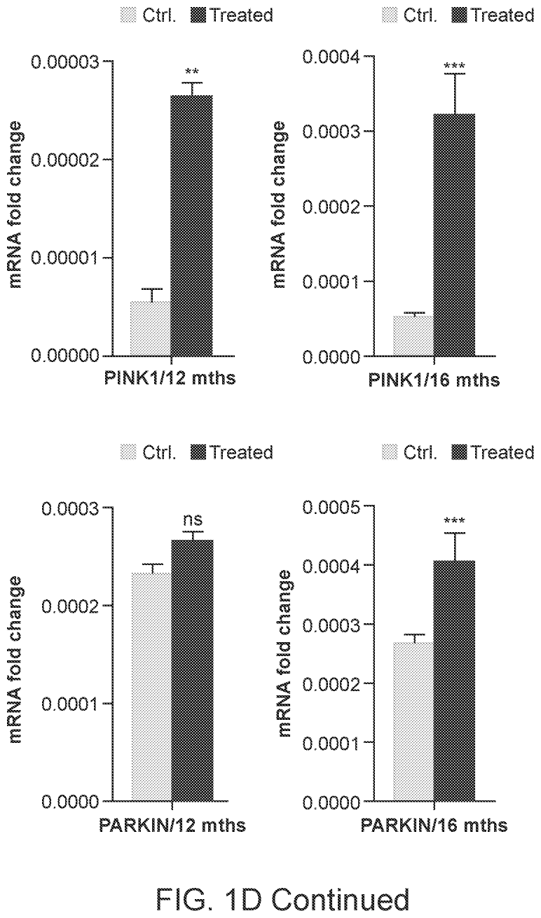

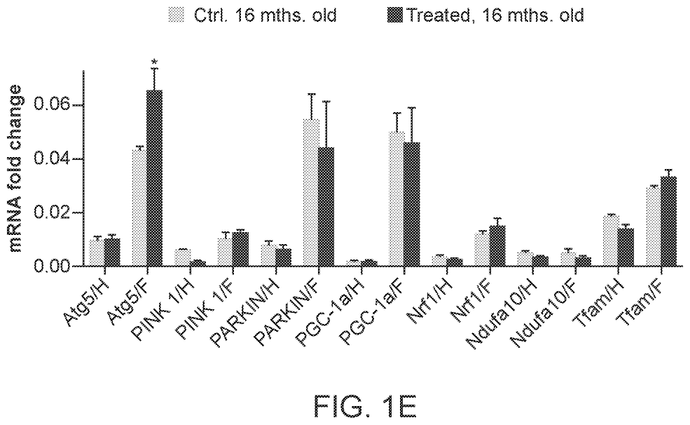

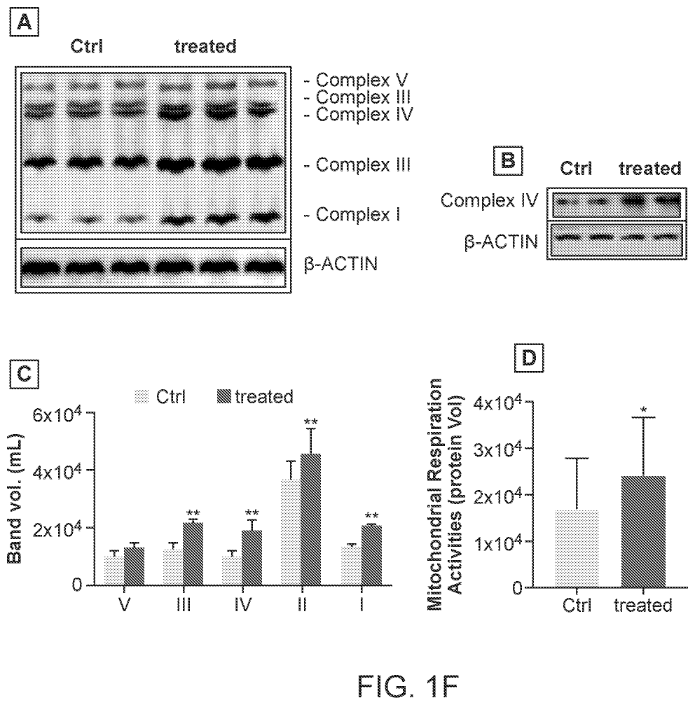

To investigate the role of the compounds on mitochondria functioning, the CcO activity between two groups of mice were evaluated. Groups of 2 and 14 month old mice were each divided into a control group and a group treated with an exemplary compound. It was observed that the activity in the 14 month old control group was halved, indicating the natural decaying process of mitochondria by ageing. In both groups of treated mice, the inhibition activity is close; 67% and 65% respectively (FIG. 1A).

The aims of the present study are to alleviate the mito-decay course, and the metabolic syndrome of ageing by tampering the respiratory operation. Thus, exploring the underneath mechanism and whether 12-month-long treatment could make any advance in function, quality and dynamics of mitochondria is of interest.

Evaluation of CcO activity in long term treated mice with a subject compound (inhibitor) vs. the single dose receivers of the inhibitor in various ages and comparing with those of controls, displayed exceptional results (FIG. 1C).

Surprisingly, in 12-month-long treated mice the CcO function is one-fold greater than the same age subjects receiving just a single dose of inhibitor. Further, it is significantly higher (about 20%) than the controls of the same model, and similar rates are demonstrated for the 14 month old mice as for the 2 month old mice after receiving a single dose of an exemplary inhibitory compound. We speculated this might be a clue of reinforced mitochondria and/or corrected dynamics in the aged group.

The ATP levels in treated mouse livers were also assessed. The decrease of 50% in 6 months, and 25% in long term treated mice, compared to controls, substantiates the moderate inhibition of mitochondria respiration but also indicates that ATP production is escalated by long term treatment (FIG. 1B).

Without being bound to any particular theory, this could be the next clue for upgraded mitochondrial quality and dynamics such as biogenesis. These findings are of interest in the ageing theory of mito-decay. To further investigate this hypothesis, the abundance levels of master transcripts of autophagy and mitophagy (Atg5, Pink1, Parkin) and mitochondria biogenesis (Ndufa10, Peroxisome proliferator-activated receptor gamma coactivator 1-alpha (PGC-1α), Sirtuins (particularly SIRT1), nuclear respiratory factors (Nrf1,2) and Tfam) among groups of 12 months and 16 months old mouse livers, treated vs. control were examined (FIG. 1D).

In this assessment, PGC-1α, and NRF1 were both increased notably in livers of aged mice. In general, mitochondriogenesis marker amplification is more distinctive in aged (16 month old) mice than 12 month old mice (FIG. 1D).

Here, SIRT1 also depicted a significant enhancement by longer course of treatment in mice (FIG. 1D). These records may indicate a CR-like mechanism that could significantly enhance stress resistance via the SIRT1 pathway (Cohen et al. (2004), Science, 305:5682, pp. 390-392), and markedly improve bioenergetics through the activation of the PGC-1α pathway (Van Diepeningen et al. (2010), Exp Gerontol., 45(7-8), pp 516-24).

We observed that mitophagy and biogenesis regulators up surged markedly through ageing in groups of 12 and 16 month-long treatment. Additionally, in the older group (16 months) this application rendered better mitochondrial function dynamically.

Mitochondriogenesis is bound to mitophagy per se. An increase of mitochondrial removal eventually leads to the biogenesis of novel mitochondria (Palikaras et al. (2015) Autophagy 11, 1428-1430).

Eliminating potentially harmful cellular debris and damaged mitochondria by autophagy and mitophagy is an adaptive survival mechanism that could avert the cell death response while allowing sufficient opportunity for the cell to replenish a healthy pool of mitochondria for sustaining energy production and cell survival. Autophagy process declines with ageing and defects in it drive oxidative stress, mito-decay, DNA damage (Ntsapi et al. (2016) Experimental Gerontology, 83:97-111; Palikaras et al. (2015) Autophagy 11, 1428-1430; and Jong-Ok et al. (2013) Nat commun., 4:2300).

It is reported that over-expression of Autophagy related 5 (Atg5) a major transcript of autophagy extends lifespan by galvanizing autophagy in mice (Ntsapi et al.). Interestingly, mitophagy can have a protective effect on oxidative damage CR-mediated autophagy that is dependent on SIRT1 increases mitophagy (Cui et al., PLoS One. 2013; 8(7):e69720); Cohen et al.; and Takae et al. (2018) Molecular neurodegeneration, 13:1: 56).

In our analyses autophagy and mitophagy markers in mice including Atg5, PTEN induced putative kinase 1 (PINK1) and Parkin showed noticeable upgrade through ageing in groups of 12- and 16-month chronic treatment (FIG. 1D). This suggests that, acting together, PINK1 and Parkin constitute a mitochondrial quality control function.

In other metabolically active organs such as white adipose and heart, only Atg5 revealed enhancement in adipose tissue and the rest showed minor variations (FIG. 1-F).

In short, these results justify that mitochondrial fidelity has been extremely maintained through ageing in mice and simulated crucial favors of CR on mitochondria.

Reversed Age-Associated Obesity

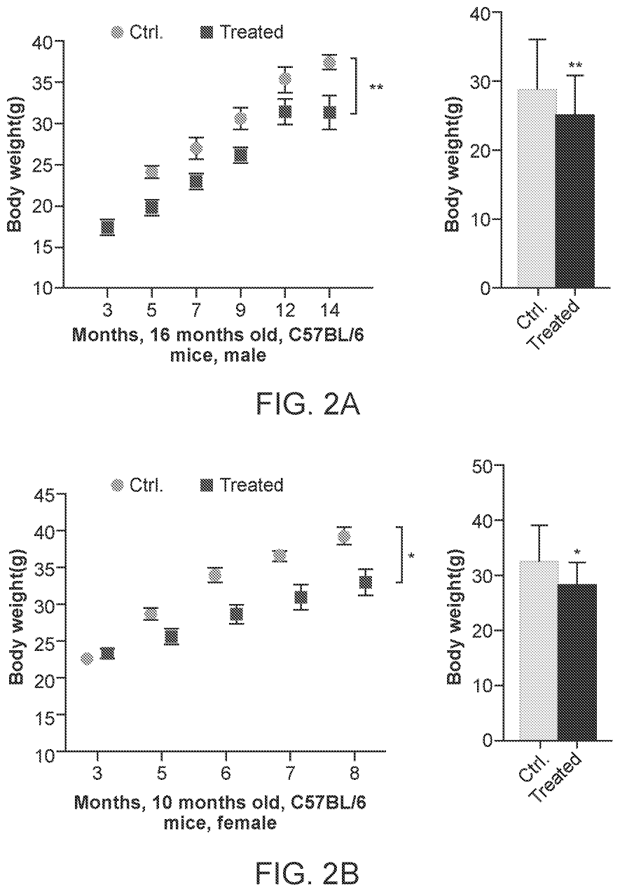

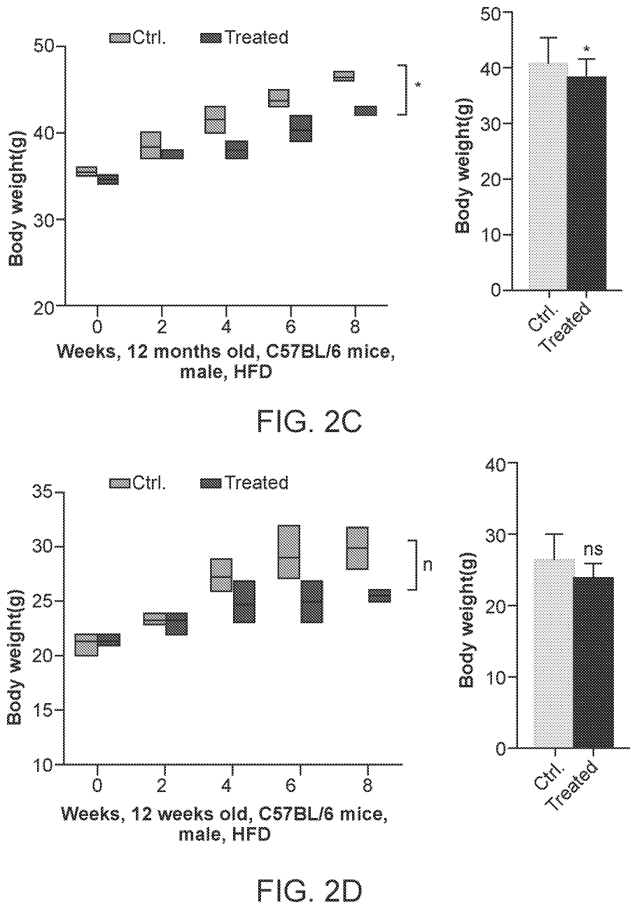

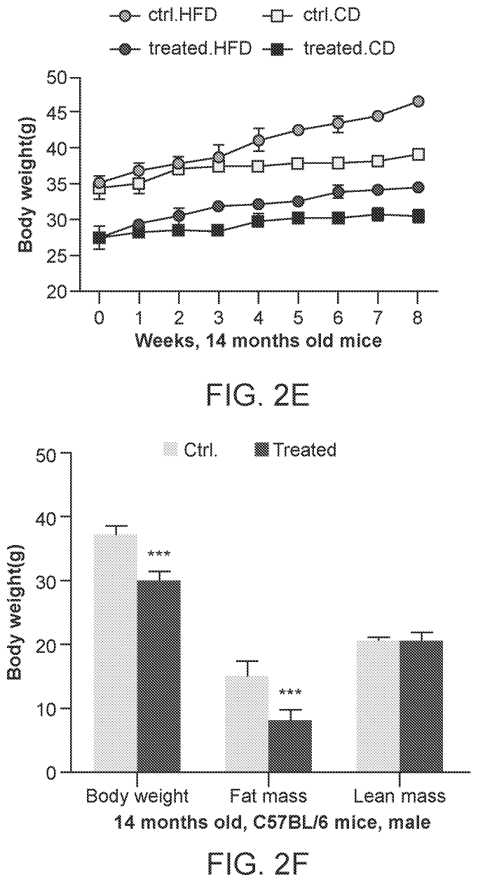

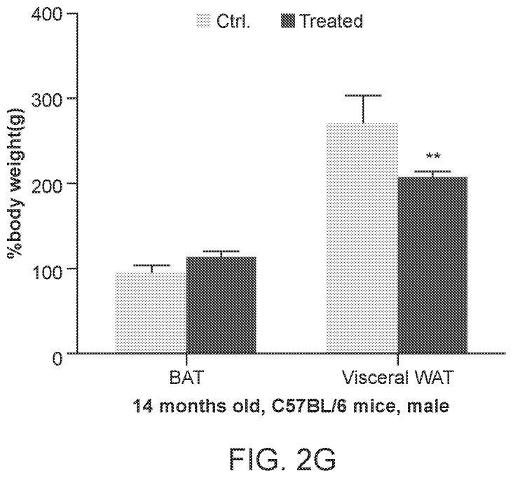

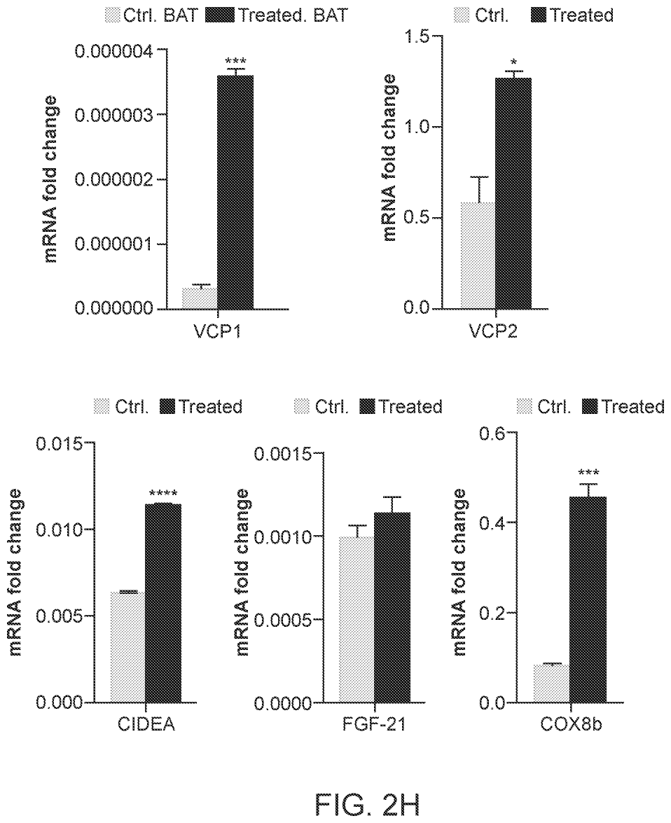

Body weight (BW) variations during a 14 month long chow diet (CD) in male mice (FIG. 2A), an 8 month long CD in female mice (FIG. 2B), an 8 week long high fat diet (HFD: 60% fat), in 12 month old male mice (FIG. 2C), and in 12 week old male mice (FIG. 2D) were monitored.

Despite catching up in length to control group of mice in early adulthood (12 weeks) in both gender, treated 14 month old male mice were significantly lighter than controls by 18% at the end of 5 months and 25% lighter after 14 months receiving the treatment. Interestingly, the female model presented greater contrast in BW variations. In treated female mice, BW were 12% less at the end of 5 months and then showed 26% decrease by 9 months (FIG. 2A and FIG. 2B).

Subsequently, we fed two groups of 12 week old and 12 month old HFD for 8 weeks, divided into two groups: a control group and a group treated with a subject compound (treatment with a subject compound had been started 4 weeks before diet changing). It was observed that the treated mice in both younger and aged groups were resistant to gaining weight and even this resistance is greater in aged group as they grow bigger, e.g., by 12% after 8 weeks HFD versus 18% in younger group. By contrast, in the control groups dramatic BW changes were observed during 8 weeks as demonstrated, e.g., 25% increase in aged group versus 20% in the younger group.

It was also observed that the BW contrast of treated vs control is 13% in the aged group and 2-4% in the younger group, at the end of 8-week-HFD (FIGS. 2C and 2D). This may indicate that young mice are less susceptible to metabolic stresses such as diet based on a more robust metabolism and better mitochondria functionality on metabolic pathways in younger subjects.

A simultaneous experiment of an 8 week HFD in 14 month old mice, which were already treated for 12 months by the subject compound, was carried out under the same conditions and compared to the CD models versus controls. This experiment manifested surprising records. Despite the markedly lower BW of the treated groups at the start of this investigation, the aged mice treated with CD defied BW gain, yet, resisted the BW gain with HFD until 5 weeks, then the increase was gradual. Controls were notably heavier by the end of HFD period (FIG. 2E).

Next, fat mass was analyzed by DEXA body composition analysis, showing recognizable decrease in body fat mass, but, trivial changes in lean mass (FIG. 2F). Alongside, analyses of subcutaneous and visceral fat (VF) depot displayed valid reduction in gonadal adipose but minor effect on sub-scapular brown adipose mass (FIG. 2G).

The inconsistency in adiposity was not associated with alterations in energy expenditure or food intake and these details may clarify the impressive effect of reversible mitochondria respiration inhibition on metabolism and age related metabolic defects like fat storage in visceral organs.

Regarding the cooling effect of the subject compound (inhibitor) in our models, in spite of fortified mitochondrial function, ignition of thermogenesis within WAT by promoted expression and activity of the uncoupling proteins (UCP1, UCP2) was of interest to consider for BW changes. As the increase of UCP1 and WAT beigeing agents, through heat production, lead to slimming (Mueller et al., Front Endocrinol (Lausanne), (2016), 7:19; Garcia et al., Nutrition & Metabolism, (2016), 13:24).

The cold inducing impact of flexible inhibition was investigated by evaluating the expression levels of main transcriptional thermoregulatory and beige markers such as Uncoupling Proteins (UCP1 and UCP2), Cell Death-Inducing DFFA-Like Effector A (CIDEA) or Cytochrome c Oxidase Subunit VIllb (Cox8b).

It appeared, UPC1 expression in brown fat of treated models was exponentially amplified by showing 20-fold increase. Similar results were observed for protein abundance of UPC1. For UCP2, CIDEA and Cox8b expression in brown fat of treated models was amplified by >one-fold, one-fold and 7-fold. These records suggest that thermoregulatory and beigeing process in visceral WAT are playing a part in the mechanism that underlies the lean phenotype (FIG. 2H).

Shrunken Visceral Adipocyte, Diminished Adiposity & Adipocyte Differentiation

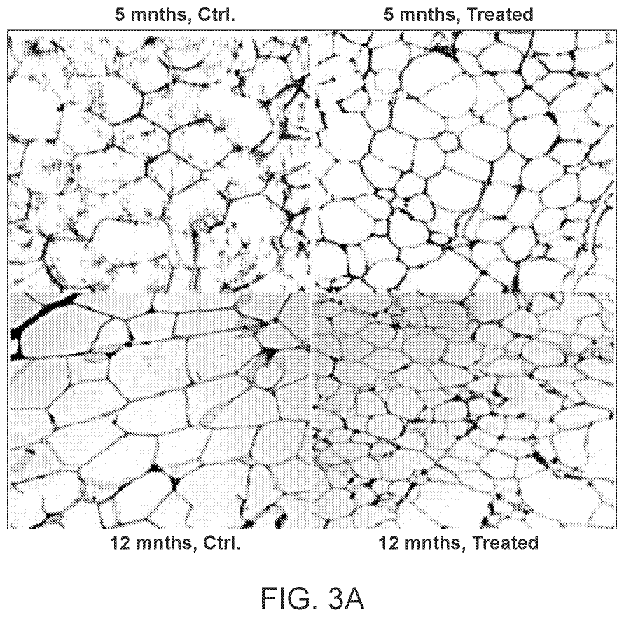

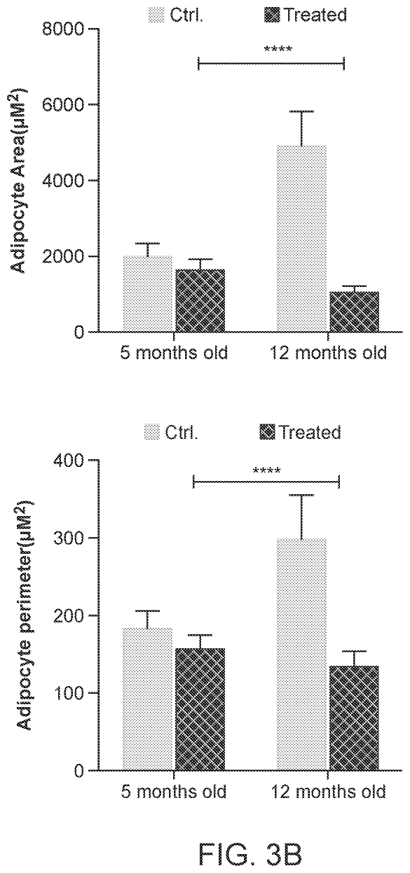

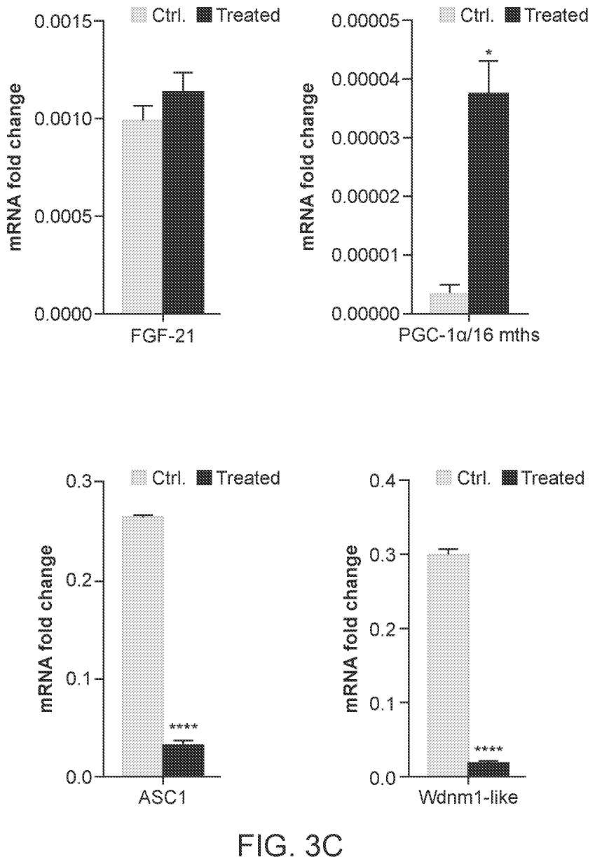

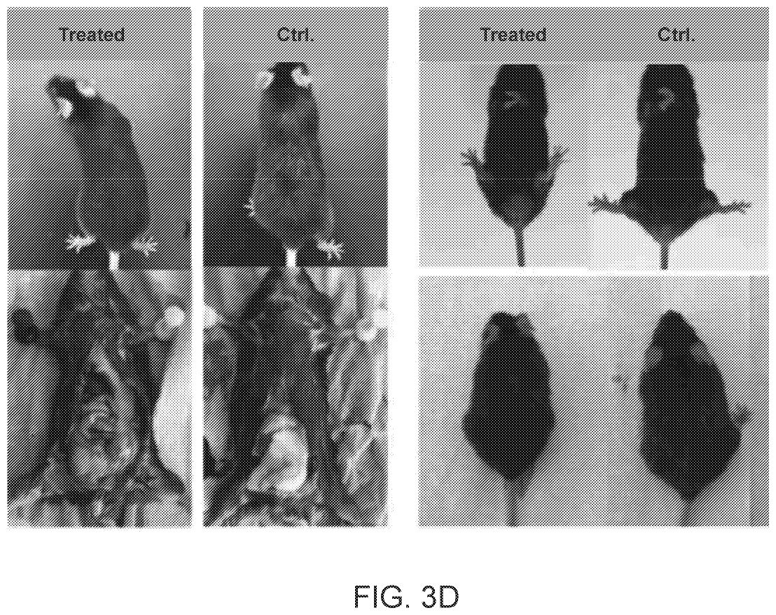

Histological analysis of white adipocyte morphology in gonadal fat pad cross-sections showed remarkable smaller adipocytes in treated mice, suggesting less mature cellular phenotypes (FIG. 3A). Adipocytes are known to enlarge during obesity and the ageing process (Zamboni et al., Mechanisms of Aging and Development, (2014), 136-137:129-37; Hemmeryckx et al., Endocrine Journal, (2010), 5710:925-30).

The increase in adipocyte size, during ageing, between two groups of mice (5 month old and 12 month old mice) was noticeable in the respective control groups. In other words, adipocyte area varied notably between two groups of treated and controls in 5 and 12 month old subjects. In treated 5 month old mice, visceral fat adipocytes showed minor changes in size and cell perimeter. This trait deters incredibly in the 12 month old model. Adipocytes in 12 month old treated mice exhibited extensive shrinkage (3-fold) in size and perimeter compared to the control group (FIG. 3B).

Next, we assessed WAT expansion transcriptional factors. As depicted, the abundance levels of the essential transcripts for white adipogenesis including solute carrier family 7 member 10 (ASC1) and WAP four-disulfide core domain-21 (Wdnm1-like) markedly decreased in the treated group by >9-fold and 30-fold than those of the control group, respectively (FIG. 3C).

Fibroblast Growth Factor-21 (FGF-21) transcript, marker for modulating glucose, lipid, and energy homeostasis and beigeing induction in WAT, remained unchanged in the treated group. It is suggested in mammals, FGF-21 is induced by multiple forms of mitochondrial dysfunction. Without being bound to any particular theory, this indicates the qualified fitness of the mitochondria in the treated group.

Further, increased PGC-1α in aged mice is another factor which may further support the anti-obesity property of this treatment (FIG. 3C). In has been found that PGC-1α mRNA expression reduces in obesity.

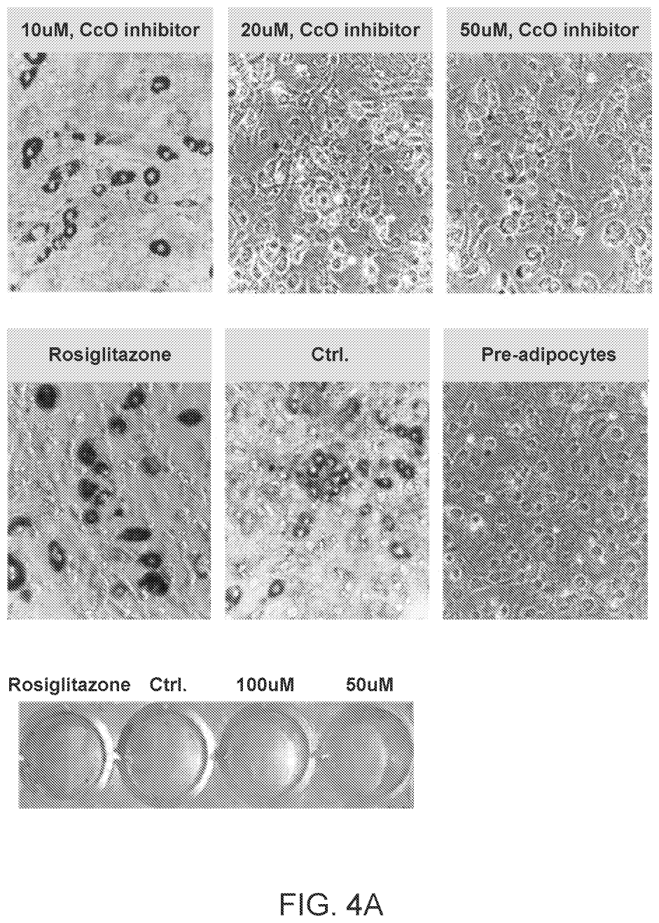

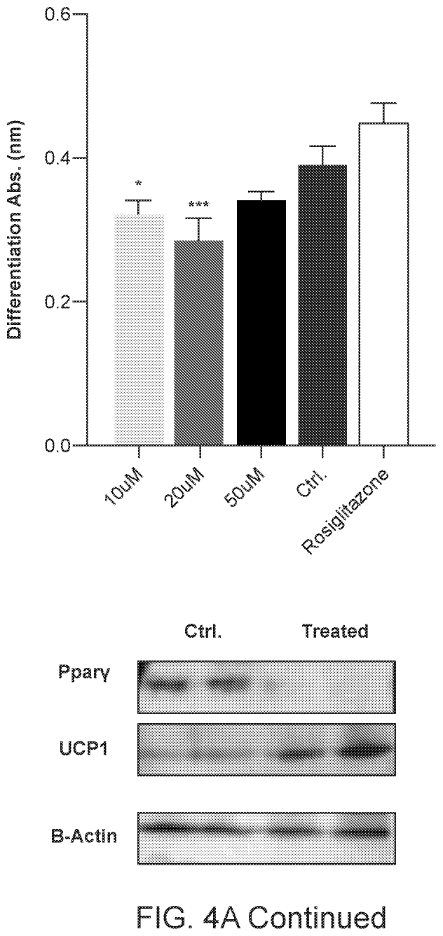

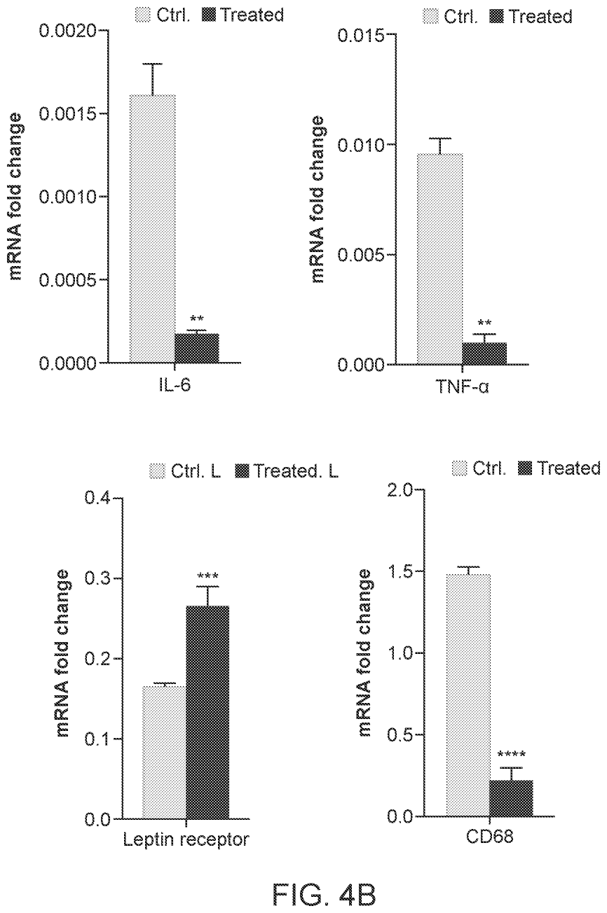

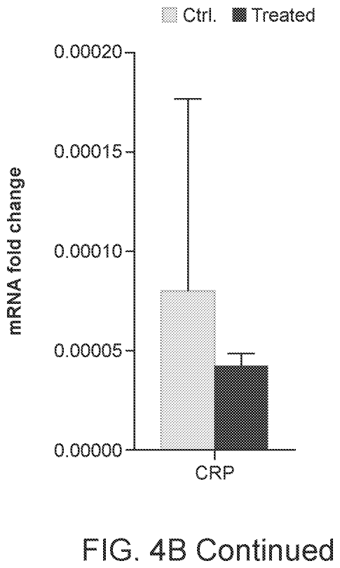

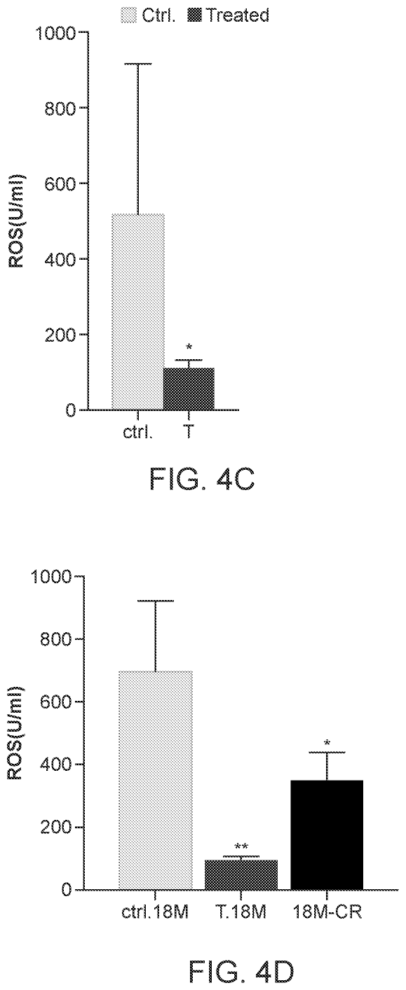

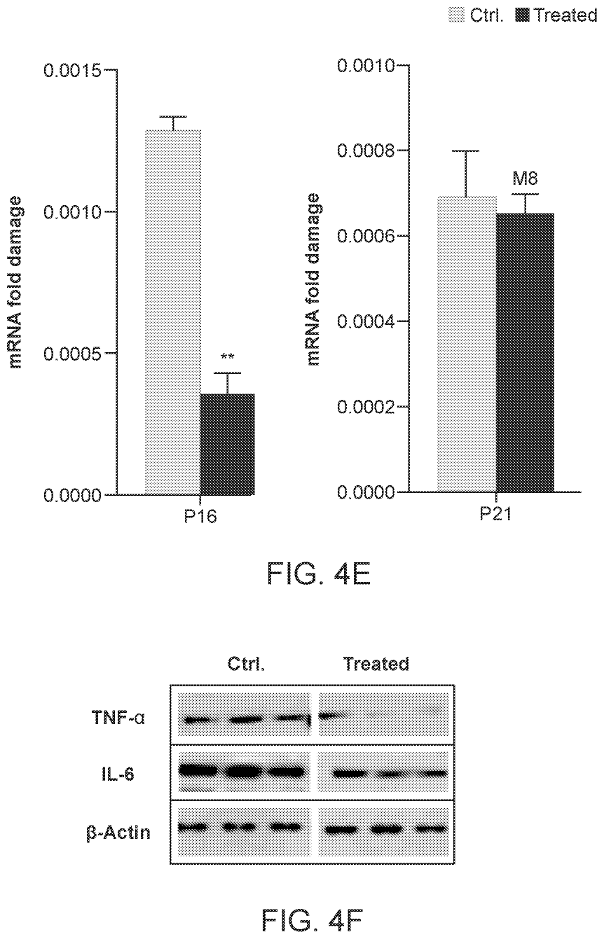

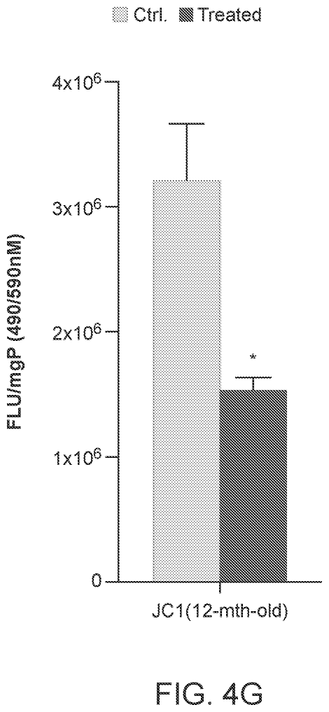

Further in vitro observations using 3T3-L1 pre-adipocytes, demonstrate that moderate mitochondrial inhibition resulted in poorly differentiated, immature adipocytes. The effect of the treatment was not due to a delay in differentiation, as treated 3T3-L1 cells incubated for up to 14 days still failed to fully differentiate into mature adipocytes. Protein abundance of Pparγ, marker indicative of mature adipocytes, shows significant reduction in cells (FIG. 4A)

In summary, these results reflect the active process of beigeing and regulated adiposity in the group treated with a subject compound. In terms of ageing adipogenesis, these results indicate that treated mice were less prone to store fat through the course of ageing.

Rectified Macrophage Infiltration & Inflammaging Markers Intensity

Inflammation is an important hallmark of aged adipose tissue (Liu et al., Horm Metab Res. (2007), 39:7, pp 489-494). Inflammatory cytokines or adipokines including Interleukin-6 (IL-6) and tumor necrosis factor-α (TNF-α), are believed to be involved in the wasting that occurs with obesity and the poor clinical outcomes seen with the metabolic syndrome (Harman, J Gerontol. (1956), 11:3, pp 298-300). Another effective factor is leptin, which acts through leptin receptor and expressed and secreted in direct proportion to fat mass. Leptin serves as a marker of energy sufficiency by rapidly decreasing during starvation and weight loss (Borden et al. J Clin Endocrinol Metab. (1996), 81, pp 3419-3423).

To better rank the variations associated with ageing inflammation, we analyzed the expression levels of TNF-α, IL-6, CRP in VF and leptin receptor in mouse livers, obtained from two groups of 14 months old mice classified as control and treated by an exemplary subject compound. As illustrated, TNF-α and IL-6 transcripts are declined markedly in the treated group (FIG. 4B). CRP which is an important clinical marker in inflammatory illnesses, remained with trivial changes (FIG. 4B).

Based on recent findings, Rats with diet-induced obesity have hyper-leptinemia and reduced expressions of leptin receptors in hypothalamus and liver (Borden et al. J Clin Endocrinol Metab. (1996), 81, pp 3419-3423). In this report aberrant leptin receptor transcript abundance (2-fold increase) in mouse livers may indicate the lower circulating serum leptin. This resembles the state of calorie restriction or starvation caused by insufficient energy (FIG. 4B) and in this study it may be justified by the role of mitochondrial inhibition in ATP production cutback.

Subsequently, the low pro-inflammatory transcript abundance in adipose tissue prompted us to assess macrophage infiltration into WAT. Infiltrating macrophages are known to be responsible for WAT inflammation, in particular the production of IL-6 (Tchkonia et al., Cell (2010), 95, pp 667-84) suggesting that WAT macrophages contribute to metabolic perturbations in this condition.

Macrophage infiltration in to WAT was detected using macrophage transmembrane pan marker CD68. Marked decline in abundance of CD68 (5-fold) is shown in treated mice compare to the control group in VF (FIG. 4B).

Ameliorated Nucleus DNA Damage, ROS Generation & Senescence Constituents in VF

Senescence and inflammation are two important mechanisms contributing to ageing and the metabolic consequences of obesity (Nguyen et al., J Biol Chem. (2007), 282, pp 35279-92).

Reactive oxygen species (ROS), DNA damage and mitochondrial dysfunction are instrumental to maintain cellular senescence (Zeyda et al., Immunol Lett., (2007), 112, pp 61-7). Since, this application dampens the mitochondrial respiration, it is expected that ROS generation will also be affected. To this end, we examined the ROS production in treated and control mouse blood plasma to investigate the impact. It was observed that ROS production dropped immensely by nearly 4-fold in treated mice (FIG. 4C). Next, we compared the ROS level of production in 18-month-old chronically treated mice with those of calorie reduction mice and a control and the results are exceptional (FIG. 4D).

Subsequently, the expressions of a few solid markers of adipocyte senescence, responsible for serious nucleus DNA damage, like p21 and p16 and IL-6, were considered. An interesting pattern is shown for expression changes of the senescence markers p16 and p21. While p16 declined in treated mice by 10-fold, p21 remained unchanged (FIG. 4E). What's more is, these findings indicate that flexible inhibition of mitochondria could be a reasonable path towards moderating ROS production and age-linked DNA damage.

Mitochondrial Membrane Potential Assay

One of the hypotheses that directs gerontological research towards accumulation of macro-molecules damaged by oxidative stress during ageing is the higher levels of the pro-inflammatory cytokines (IL-1αβ, TNF-α, IL-6) and adipokines (Leptin) with persistence of the inflammatory infiltrate of macrophages within the tissues induced by ROS (Rimessi et al., Int J Biochem Cell Biol., (2016), 81(Pt B), pp 281-293). Evidence suggests that the mechanisms by which ROS induce chronic inflammation relies on the ROS ability to activate the cell signaling cascade that is considered a master regulator for the expression of several pro-inflammatory genes (Rimessi et al.; Mirsoian, et al. Journal of experimental medicine, (2014), 211, 12). In calorie reduction the anti-inflammatory effect is a passive mechanism linked to the reduction of inflammatory stimuli such as ROS, and also exerts active and positive actions on metabolic gene expression products that repress pathways of inflammation in several types of tissues including liver, hearth, muscle, white adipose tissue in addition to critical inflammatory defects (González et al. Oral Diseases, (2011), 18:1, pp 16-31; Arlia-Ciommo et al., Oncotarget (2018), 9:22, pp 16163-16184; Redman et al. Antioxid Redox Signal., (2011), 14:2, pp 275-87). Reports suggest, mitochondrial membrane potential (Δψm) is a central bioenergetic parameter mainly controlling the generation of ROS, and calorie restriction prevents mitochondrial membrane hyperpolarization, reduces Δψm and ROS, which means an eventual lower rate of oxygen consumption or ATP production (Van Diepeningen et al., Exp Gerontol. (2010), 45:7-8, pp 516-24; Zorova, et al., Anal Biochem., (2018), 552:50-59).

To investigate this theory, we assessed the mitochondrial membrane potential (Δψm) signal by using 5,5,6,6-tetrachloro-1,1,3,3-tetraethylbenzimidalolylcarbocyanine iodide (JC1) in livers of 12 month old mice treated for 10 months (FIG. 4G). We noticed our records highly mimic the calorie restriction response by producing very efficient electron transport through the respiratory chain, that is equivalent to ATP production. Without being bound to any particular theory, this change in mitochondrial efficiency could attenuate molecular and cellular damage resulting from oxidative stress and therefore, reduce the rate of ageing at cellular and organismic levels.

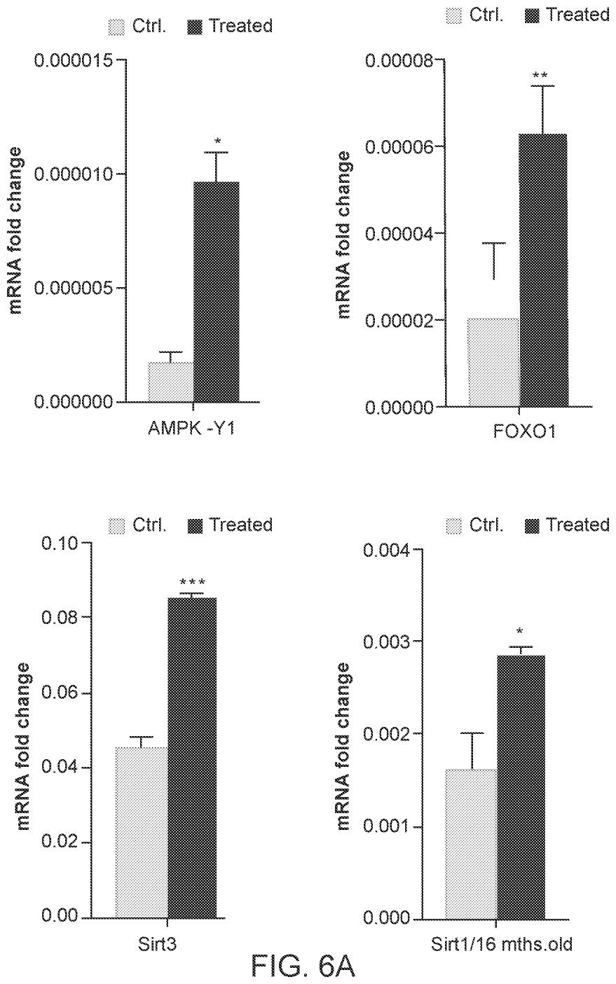

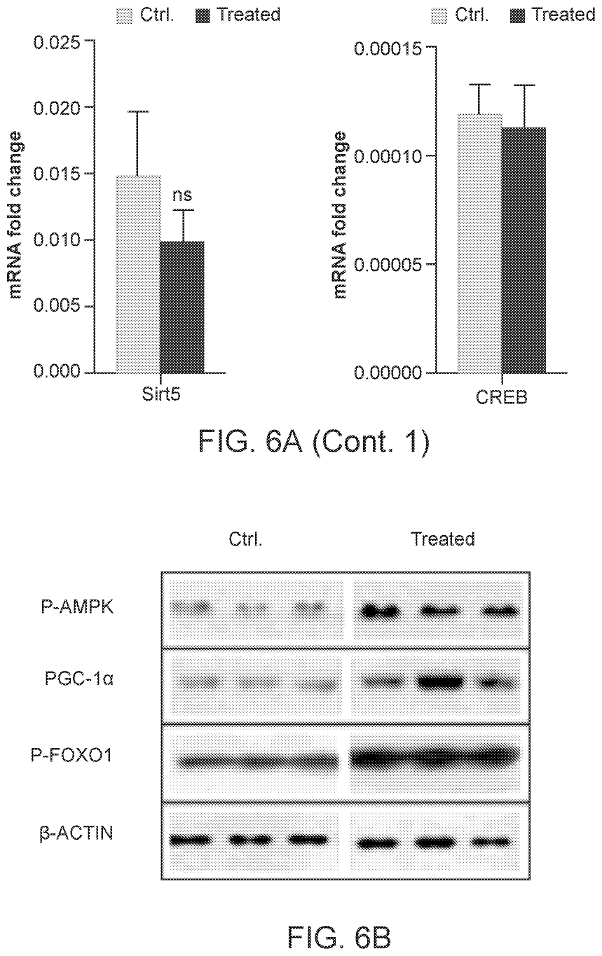

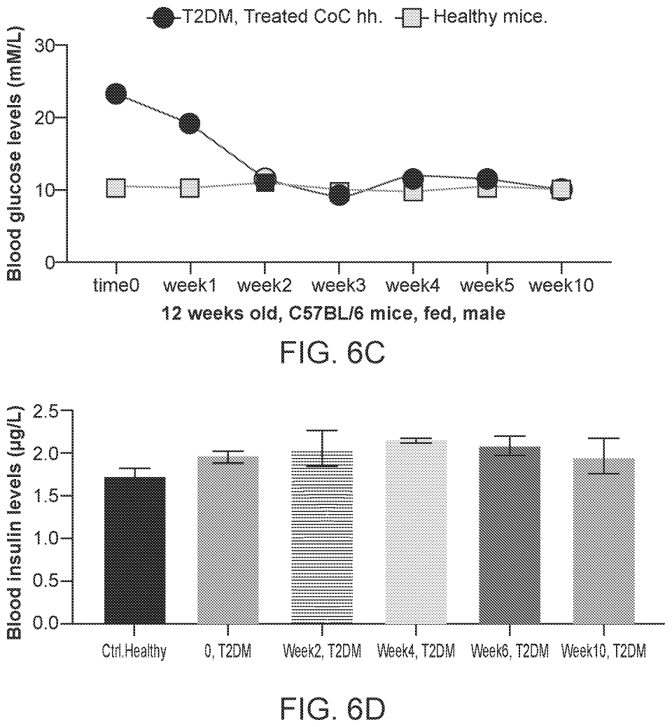

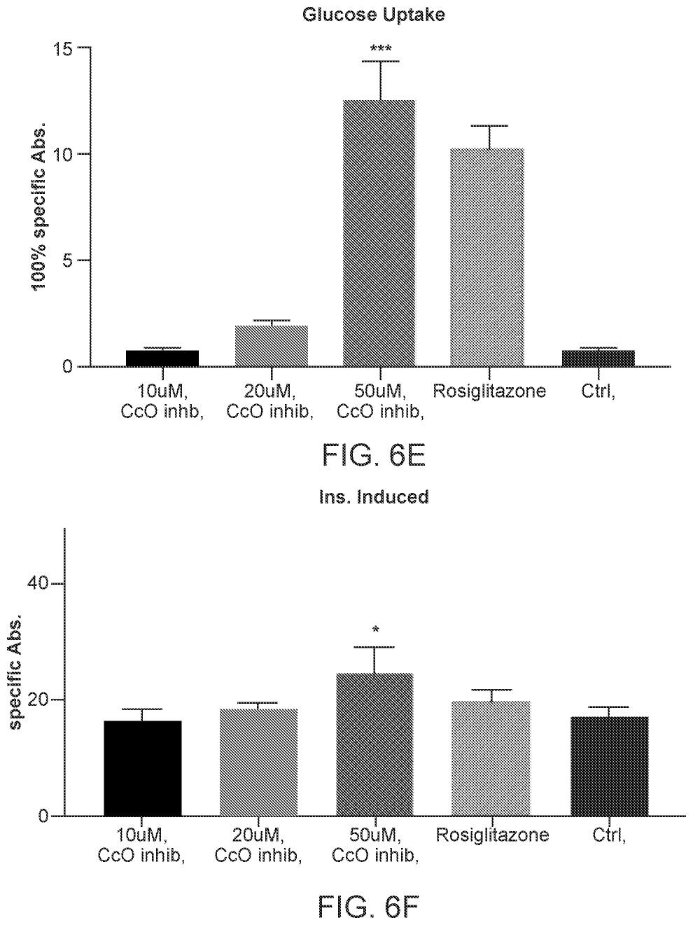

Of note, SIRT3 is also considered to play a substantial role in calorie reduction-induced longevity. As disclosed herein, SIRT-3 demonstrated a marked upgrade in the disclosed treated group (FIG. 6A). In summary, this data suggests that treatment with a subject compound may be more effective for anti-ageing than calorie restriction.

Enhanced Insulin Sensitivity, Energy & Glucose Homeostasis

Insulin resistance (IR) represents a major component of metabolic syndrome and is commonly observed in obese older adults (Ford et al., JAMA., (2002), 287, pp 356-359).

We explored the effect of the compounds on glucose homeostasis and insulin sensitivity. Thereupon, we evaluated glucose homeostasis and insulin sensitivity in two classes of treated and control mice. In the current study, we found that the group of mice treated with 100 mg/Kg per day showed lower glycated hemoglobin (Hb1Ac) levels after 12 months continuous treatment compared to their control and calorie restriction group counterparts (Table 1).

TABLE 1
Effects of moderate inhibition on various serum biomarkers
in mice compared with control and calorie restriction model

| | | | Calorie |
|---|---|---|---|
| Parameter | Treated | control | restriction model |
| Total protein (g dl^{□-1}) | 5.89 ± 0.32 | 6.09 ± 0.39 | 5.28 ± 0.04* |
| Glucose-fed (mM) | 6.5 ± 0.3* | 8.5 ± 0.5 | 7.5 ± 0.5 |
| Glucose-fasted (mM) | 4.4 ± 0.2* | 6.5 ± 0.5 | 5 ± 0.6* |
| Insulin (ug/L) | 2.34 ± 0.5* | 2.78 ± 1.8 | 2.08 ± 0.46* |
| % HbA1c | 5.5 ± 0.5* | 6.5 ± 0.47 | ND |
| Cholesterol (mM/L) | 6.3 ± 0.9 | 6.2 ± 1.0 | 6.00 ± 1.1* |
| Triglycerides (mM/L) | 1.00 ± 1* | 1.4 ± 0.9 | 1.05 ± 1* |

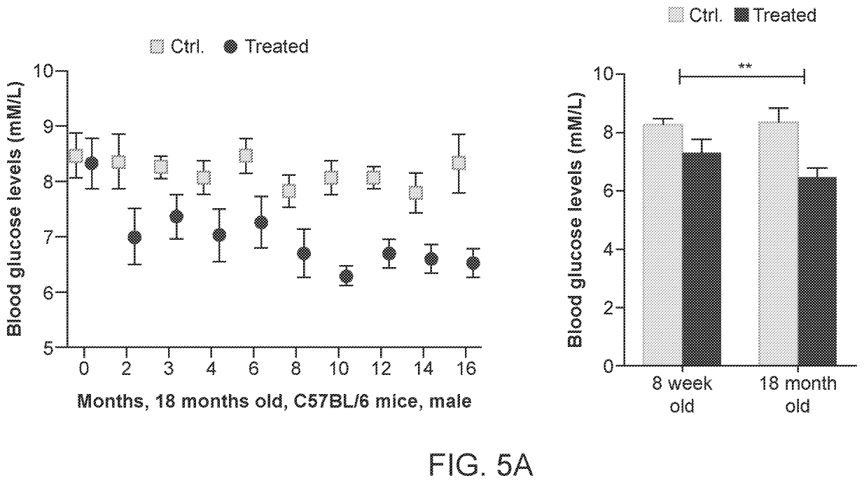

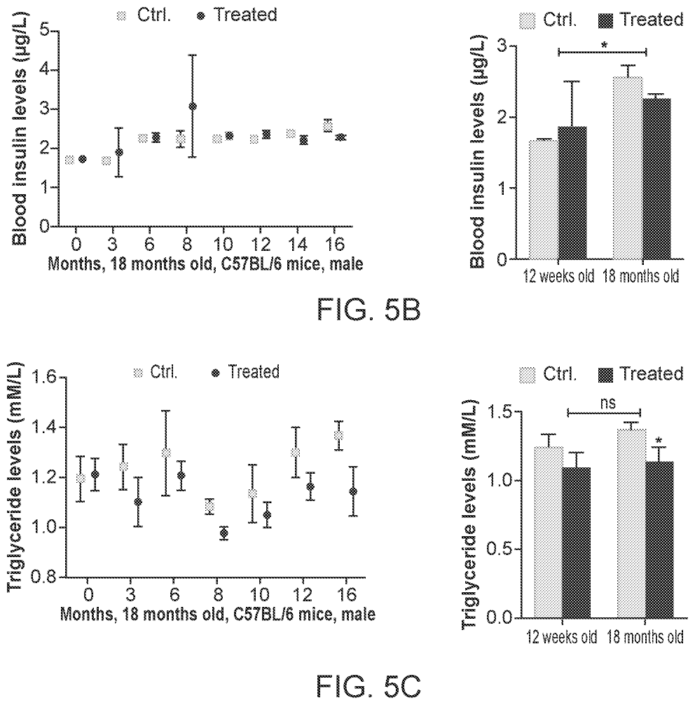

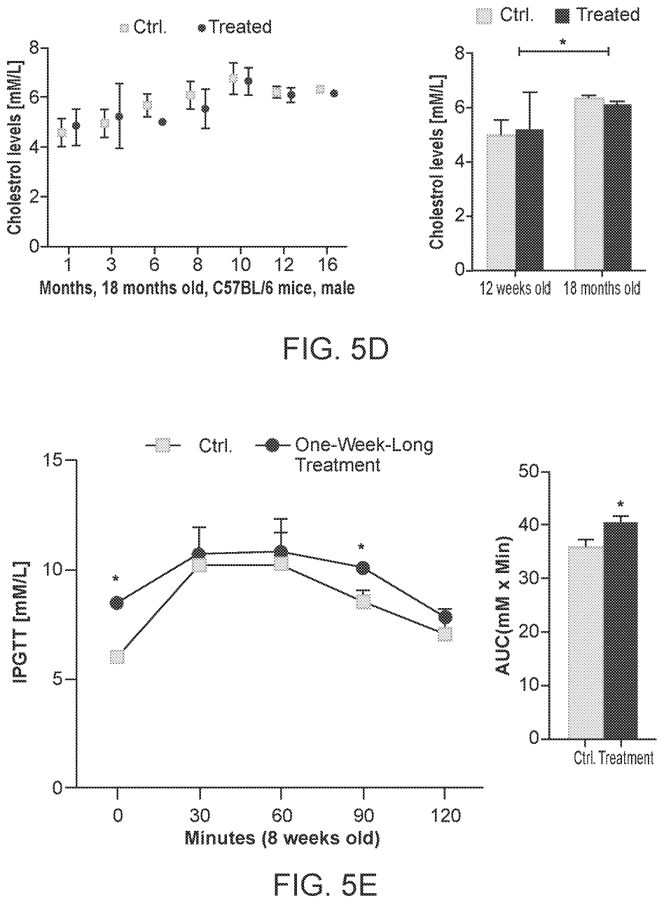

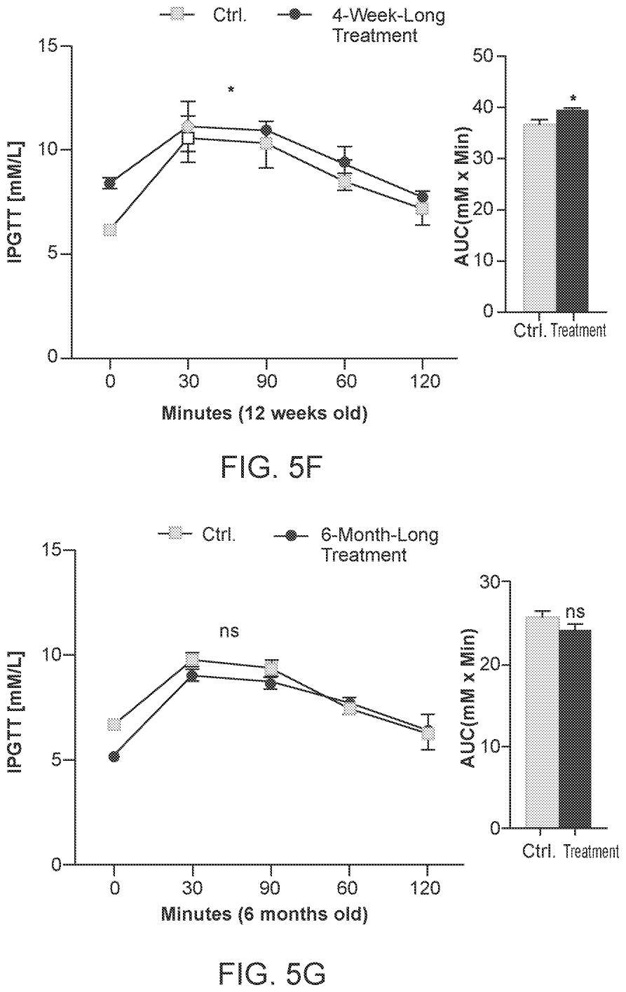

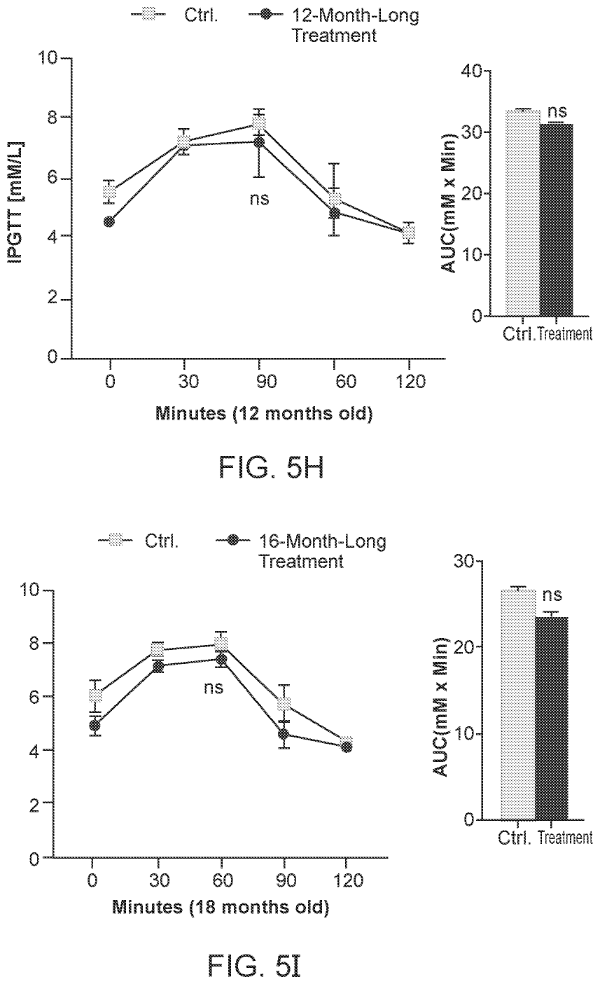

Mice treated with an exemplary subject compound displayed comparable glucose homeostasis (FIG. 5A) when measured by intra-peritoneal glucose tolerance test (IPGTT) at 18 months of age (FIG. 5E-5I). Mice treated for 18 months improved markedly in serum metabolite levels that are associated with diabetes compared with those of controls and calorie restriction model mice, showing a reduction in insulin levels, TG and the glucose (Table 1 and FIG. 5A-D). Free fatty acids and triglyceride (TG) levels show better plasma lipid profiles in treated models of both ages (FIG. 5C). Hence, circulating plasma cholesterol variations in all models are insignificant (FIG. 5D).

Simultaneously, IP glucose tolerance tests at different ages revealed that treated mice at young ages of 8 weeks and 12 weeks are mildly glucose-intolerant and exhibit mild systemic IR (FIG. 5E and FIG. 5F). This may be caused by the bodies reaction to the treatment at the beginning of the study. As depicted, after 6 months, mice show normal glucose tolerance or IR levels and demonstrate better status than controls (AUCs) (FIG. 5G-5I).

Taken together, these results suggest that mild inhibition prevents the onset of metabolic syndrome. Inventors hypothesize that the mechanism underneath the homeostatic character can be explained by the ensuing perturbations in cellular energy status, and redox homeostasis that increase the AMP/ATP ratio, that in turn, result in activation of the energy sensor AMPK which plays an important role in regulation of energy metabolism (Kim et al., Diabetes, (2008), 57:2, pp 306-314), and interferes with hepatic gluconeogenesis. Hepatic gluconeogenesis is important for maintaining blood glucose levels. In conditions like fasting or caloric restriction AMPK activation can prevent gluconeogenesis in liver by phosphorylating, inducing the course of deacetylation and activation of transcription factor FoxO in the nucleus then result in the expression of gluconeogenic enzymes. As shown, in the livers of treated mice the relative levels of phosphorylated AMPK increased by 5-fold and led to enhanced deacetylation of FoxO-1, by 3-fold. Protein assessment of these markers exceedingly emphasizes the resemblance to some extent those of calorie restriction. (FIG. 6A and FIG. 6B).

Next, in a biased investigation on impact of partial inhibition as a potential anti-diabetic treatment, two groups of type 2 diabetes mellitus (T2DM) and healthy control mice were prepared. Four weeks after the verification of T2DM, diabetic mice began to receive the exemplary subject compound. The chronic treatment phase lasted for 10 weeks.

At time 0 the average glucose levels of diabetic model were 23 mM. After one week the elevation dropped by 20% and by week 3 they reached to healthy control subjects (39% drop) and stayed stable. At this point, insulin elevations were negligible (FIG. 6B and FIG. 6C).

Meticulous side by side comparison of records to understand the underneath mechanism responsible for the hypoglycemic trait of inhibition led to few observations. First, analysis of major transcripts associated with hepatic glucose metabolism and IR such as AMPK, Forkhead box protein O1 (FOXO1), SIRTs and cAMP response element-binding protein (CREB), revealed that the anti-diabetic character of flexible mitochondrial respiration inhibition in treated mice might be an indication of gluconeogenesis prevention by AMPK activation which also caused boosted abundance of Forkhead box protein 01 (FOXO1) and SIRT3 in mouse livers (FIG. 6A).

Hepatic gluconeogenesis is important for maintaining blood glucose levels. AMPK activation can inhibit gluconeogenesis by phosphorylating, induce deacetylation and activation of transcription factor FOXO in the nucleus then results in the expression of gluconeogenic enzymes during fasting (Redman et al., Antioxid Redox Signal, (2011), 15:14(2), pp 275-87) or caloric restriction (Masternak et al., PPAR Research, (2007), 28436).

Second, adipocytes are the main site of insulin action and, thereby, play an important role in glucose metabolism as well as in the regulation of body glucose homeostasis. Therefore, the glucose uptake tests were evaluated on matured adipocytes to stimulate 2-NBDG (glucose) in the absence and presence of insulin and Rosiglitazone (insulin-sensitizer) and the higher insulin sensitivity was verified after treatment by the inhibitor. Interestingly, it was discovered that 2-NBDG uptake by an inhibitor was notably higher than the insulin (control) and Rosiglitazone at its highest concentration i.e. 50 μg/mL (FIG. 6D and FIG. 6E).

It is reported that TNF-α and IL-6 escalation and the decline of UCPs in adipose tissue are among the factors that have been associated with IR and type 2 diabetes (Alessandro et al. (2016) Transient rapamycin treatment can increase lifespan and healthspan in middle-aged mice. eLife vol. 5 e16351. 23 August).

Augmented Life-Spanning & Youth Markers.

Some particular transcripts like AMPK, FOXOs, Sirtuins exert pro-longevity effects in a diverse range of species. The AMPK-FOXO pathway plays an important role in the ability of a dietary restriction regimen to extend lifespan in Caenorhabditis elegans (Mirsoian et al., Journal of experimental medicine, (2014), vol. 211:12, 2373). It seems that AMPK signaling is an important regulator of health and life-span (Gonzalez et al., Oral Diseases, (2011), 18:1, pp 16-31). Besides, mitochondrial sirtuin3 (SIRT3) has received much attention for its role in metabolism and ageing. Specific small nucleotide polymorphisms in SIRT3 can be linked to increased human lifespan (Siegmund et al., Human Molecular Genetics, (2017), 23:1, pp 4588-4605).

In summary, we examined these several markers to assess the anti-ageing properties of exemplary subject compounds. It was observed that, APMK, FOXO1, SIRT3 are amplified 5, 3 and one-fold respectively (FIG. 6A).

Discussion

Ageing is arguably the most universal contributor to the etiologies of metabolic decline and related diseases and is associated with progressive loss in mitochondrial function. Mitochondria are the major source of ROS generation, which can lead to oxidative damage and poor functioning. Thus, mito-dynamics and quality control represent a potential valuable approach for the development of new therapies for those diseases which course with mitochondrial damage and/or inflammation.

The goal of the present study was to harness the potential of reversible/flexible inhibition of CcO to retard the mito-decay process and extend the healthy ageing by ameliorating metabolic syndrome.

In summary, our treatment model reveals a level of chronic CcO moderate inhibition exposure that can expand health span and tackle the deleterious effects of ageing in mature male mice remarkably. The results discussed herein show that the organism responds to a low-energy challenge by minimizing anabolic processes (synthesis, growth, and reproduction), favoring maintenance systems, and enhancing stress resistance, tissue repair, and recycling of damaged molecules. Likewise, the present study could harness the potential of extending health span by a compound-based mitochondrial respiration moderation and introduce a confident replacement for dietary restriction. Indeed, this intervention could simulate caloric restriction anti-ageing traits extensively, even though food intake remained unchanged in all of our models.

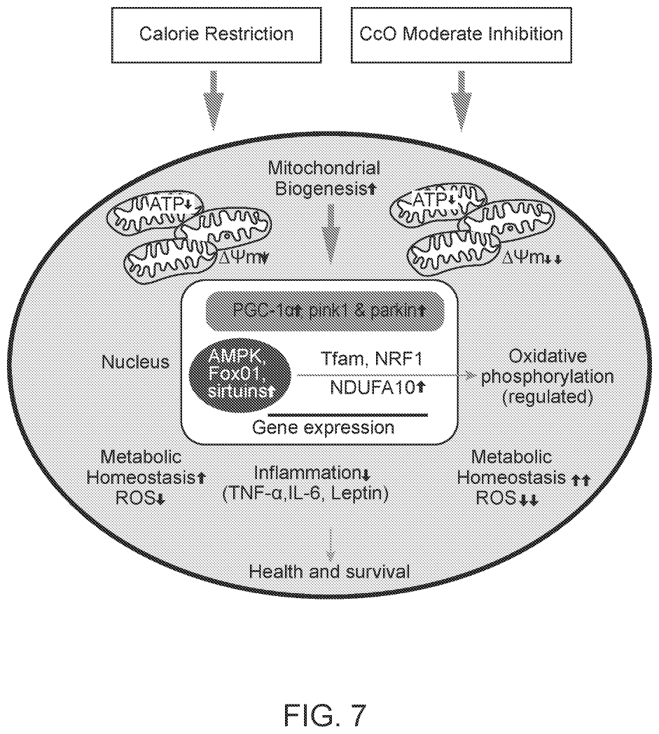

We were able to demonstrate that an 18 month long regulated mitochondrial inhibition in mice was well-tolerated without any deleterious effect. While it is clear that mitochondrial respiration inhibition partially inhibits oxidative phosphorylation, we have found no evidence for this with long-term exposure in vivo, suggesting that adaptation to treatment with a subject compound occurs and is associated with benefits and CR striking characters of reversing ageing (FIG. 7). Herein it is demonstrated that long term regulated inhibition could tune up mitochondria function notably by amplifying mitochondriogenesis markers (PGC-1a) and mitophagy activators, such as PINK1 and Parkin. Peroxisome proliferator-activated receptor gamma co-activator-1a (PGC-1a) which has been extensively described as a master regulator of mitochondrial biogenesis, demonstrated a significant change by this application.

Hence, the mitochondrial theory of ageing proposes that mito-decay motivated by reactive oxygen species (ROS), is a major cause of cellular energy decline. Chronic treatment of mice with a subject compound could shape up the electron transport very efficiently and result in a grave decrease of damaging factors and ROS compared with those of control and calorie restriction groups.

After long term regulated treatment, the treated group were notably lighter than controls exhibiting age-related and high-fat diet-induced anti-obesity property without metabolic dysfunction. The WAT inducing transcripts (ASC1, Wdnm1-like) and beigeing factors (UCP1, UCP2, CIDEA and COX8b) represented marked fluctuations in VF depot in treated mice which indicate regulated adipogenesis and healthy fat mass accumulation. Additionally, increased leptin receptor expression was observed. High leptin receptor transcript levels signify lower concentration of serum circulating leptin and accentuates the anti-age-linked obesity character.

Also, major adipokines associated with metabolic syndrome faced impressive reduction in VF. Decrease of vital inflammageing factors (TNF-α and IL-6) demonstrates that proinflammatory markers, which are associated with ageing, are refined by the disclosed treatment. TNF-α and IL-6, can contribute to the pathogenesis of IR and its age-associated chronic conditions.

The lowered pan macrophage marker (CD68) and enhanced autophagy (Atg5) demonstrates healthier cells and less left decayed intercellular organelles such as mitochondria. Macrophages are highly specialized in removal of dying or dead cells and cellular debris. This role is important in chronic inflammation, as the onset of age linked inflammation and DNA damaging is coming along with increase of macrophage markers. Hence, Autophagy has been implicated in the ageing process and suggests that overexpression of Atg5 in mice activates autophagy and extends lifespan.

We also demonstrated that CR-like, chronic exposure to a compound rectified energy homeostasis and this alteration caused by activation of AMPK. Following, enhanced FoxO1, SIRT1 and SIRT3 transcripts optimized the homeostatic regulations and longevity phenotype. There are alterations in hepatic AMPK activity during normal ageing in mice. It follows that major impact on hepatic lipid and glucose metabolism and energy production is expected when comparing young and old animals.

Regarding energy homeostasis, our records indicate that moderate mitochondrial inhibition can reverse age-related and diet-induced obesity, independent on changes in glucose tolerance, insulin sensitivity or lipid profile. This suggests that dampening mitochondria respiration is an unorthodox method that may preferentially drive the healthy glucose homeostasis.

In summary, our data demonstrate the life/health spanning trait of reversible mitochondrial respiratory inhibition and intriguingly hint that this application has a preventive and therapeutic potential as an effective antagonism of metabolic syndrome, and it might conjure up a new perspective with respect to the treatment of aging. Also, this data suggest that the subject compounds have potential therapeutic application as an alternative to calorie restriction.

Methods & Materials

Animal experimentation. Male C57BL/6 mice (Shanghai SLAC Laboratory animal Co. Ltd) were group-housed in a barrier facility with 12-hr light/12-hr dark cycles. All mice received a regular chow diet ad libitum (PicoLab 5053 Rodent Diet 20; Lab Diets) except for HFD (40-60%) and CR models. The compound was then administered in drinking water ad libitum at either 90-100 or 100-120 mg/kg/day, based on the previously measured water consumption.

The C57BL/6 mice were maintained on a standard purified mouse diet (PicoLab 5053 Rodent Diet 20; Lab Diets) until they reached 2 months of age when the treatments started. The CR animals were subjected to a lifelong restriction on PicoLab 5053 Rodent Diet 20; Lab Diets, starting at 12 weeks of age, with a daily food allotment of 60% of that eaten by the ad lib animals. Besides CR animals, the C57BL/6 groups in this study were fed the standard high fat diet (HFD; 60 or 40% fat). Body weights were measured weekly at the same time each week. Food and water intake were measured once every month for 3-6 consecutive days at the same time each month. Water consumption was measured for 2 weeks prior to the start of administration. Compound was generated and modified for better permeability in to mitochondria, evaluated by HPLC, was 96%-97%. The administration began at 8 weeks of age and continued for 16 months, until they became 18-month-old. The drug solution was prepared weekly in small batches by dissolving the compound into autoclaved water at the respective doses and filtering sterilely. Aliquots were collected from each batch and measured by HPLC to confirm stability of the compound. Water bottles and cages were changed twice weekly. Fed and fasted blood samples were collected monthly at the same time each month from tail and used for glucose, insulin, blood cell counts, lipid analysis using assay kits (Robio, China) and HbA1C assay kit (Crystal chem, USA) following the manufacturer protocol. IPGTTs and ITTs were conducted throughout morning and early afternoon after fasting for 16-18 hrs. and 4 hrs., respectively, once before start of treatment administration began and every 3 months thereafter. Rectal body temperature was measured monthly, as described previously. Whole blood samples, and histopathology were analyzed after 14 months of treatment. Tissue samples were collected after 10-14 months from mice, sacrificed and used for WB and qPCR microarray, and other analyses. All animal studies were approved by the Shanghai Jiao Tong University Animal Studies Committee and were in accordance with NIH guidelines. (Number of mice; SD=30, HFD=10, CR=20).

Dose determination in mouse. Based on IC50 (Di Francesco et al., Science, (2018), 362, 770-775), initially we created an effective window for the inhibitor. We considered the minimum effective dose is equal to IC₅₀ thereafter, began to inject the inhibitor and gradually increased the dose (+50 per intrapritoneal (IP) administration, 3 mice on consecutive days) until the peak was reached when the experimental model (C57BL/6, male mice) showed dramatic decreased body temperature. The selected oral dose with the least side effects used during the whole experiment, was around 90-100 mg/kg.

Body temperature integration in mouse. Three groups of male C57BL/6 mice received a single injection of the inhibitor at dose-levels of 0 (vehicle=N/S), average μM and max μM and were then kept for an 80 minute observation period until the BT was stabilized. Treatment was performed as a slow injection, IP, under a dosage volume of 0.5 mL/kg.

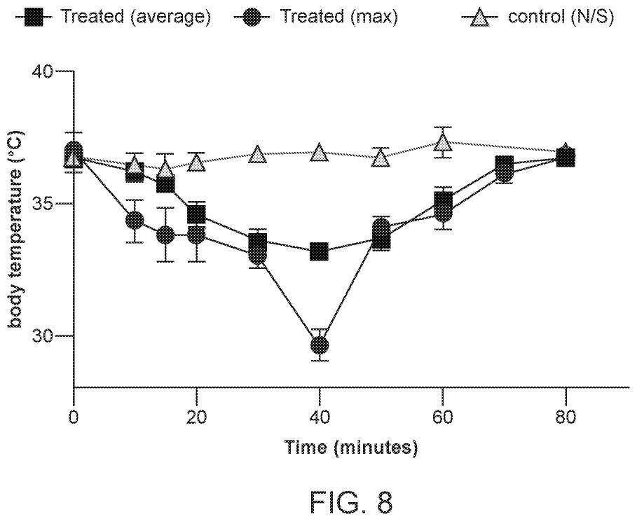

Rectal temperature was measured using a conventional thermometer in all groups at 0, 10, 15, 20, 30, 40, 50, 60, 70 and 80 minutes and repeated three times in 3 days. Clinical pathology (physicals) 24 h after treatment (Day 4) was within the normal baseline range and changes in these parameters were trivial in both treated groups (FIG. 8).

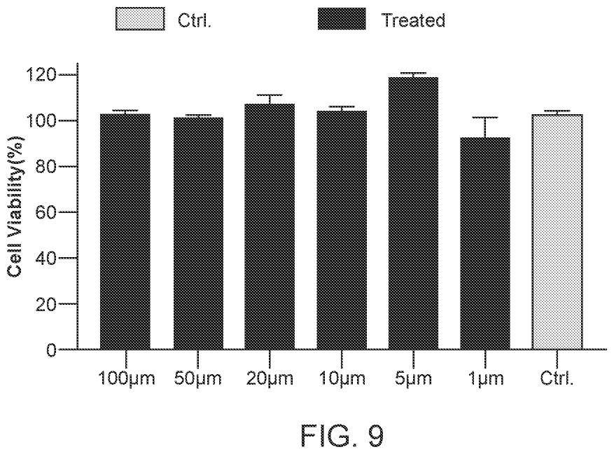

MTT assay and toxicity assay of the compound. Carefully aspirate the media from Hep-G2 culture. An alternative method is to add an equal volume of MTT solution and our compound to the existing media in the culture. Ensure that the same volume of existing media is present for each sample. Otherwise add 50 μL of serum-free media and 50 μL of MTT solution into each well plus various doses of inhibitory compound to each well, triplicate each sample. Incubate the plate at 37° C. for 3 hours. After incubation, add 150 μL of MTT solvent into each well. Wrap plate in foil and shake on an orbital shaker for 15 minutes. Occasionally, pipetting of the liquid may be required to fully dissolve the MTT Formosan. Read absorbance at OD=590 nm. Read plate within 1 hour. (FIG. 9)

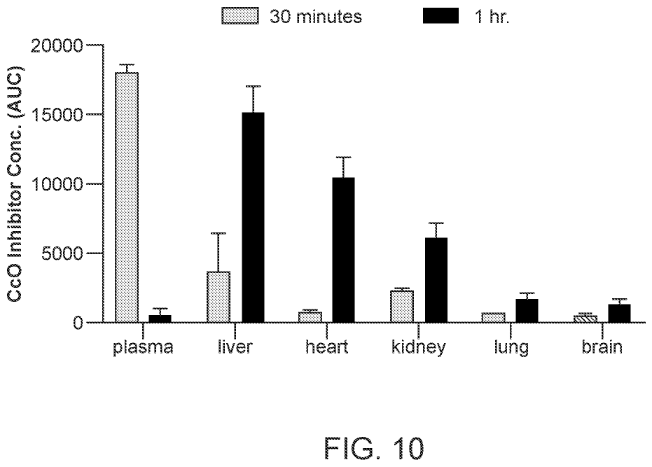

LC-MS analyses. Organs were homogenized and diluted in isotonic NaCl solution. Protein was precipitated using acetonitrile containing internal standard and analyzed. The analysis was carried out using gradient condition with mobile phases consisting of aqueous phase and pure acetonitrile. Analysis was run at a low-rate of 0.51 mL/min. The method was selective with a limit of quantification of 0.5 μg/mL in homogenate at a sample volume of 100 μL. The standard curve was linear over a concentration range of 0.5-10 μg/mL for all organs (FIG. 10)

Fluorescent microscopy. Adipose tissue was harvested and fixed in 4% paraformaldehyde (SigmaP6148). Adipocyte size was measured by staining for toluidine blue O in 5-20 μm-thick sections, paraffin embedded (Cell Signalling Technology, Tissue-Tek O.C.T.). adipocyte differentiations assessed by Oil Red 0 staining.

Measurement of ATP level in liver. For measuring ATP, cellular ATP in livers was extracted using an ATP extraction kit of an ATP assay kit (Sigma-Aldrich, MAKI 90). The ATP level was quantitatively measured by the absorbance at 570 nm (A570).

Measurement of CcO activity in liver mitochondria. Mouse livers were extracted, and liver mitochondria were obtained using a mitochondria isolation kit (Sigma-Aldrich). CcO activity in liver mitochondria was measured using a cytochrome c oxidase assay kit (Sigma-Aldrich, CYTOCOX1, USA.

Immunoblotting analyses. Western blot analyses carried out in mouse livers, heart and adipose tissues collected from aged mice after treatments. Proteins (40 μg) were separated by electrophoresis on a sodium dodecyl sulfate (SDS) 4-20% polyacrylamide gel under reducing conditions. Membranes (Amersham Hybond PVDF, Germany) were probed overnight at 4° C. using Total OX-OPHOS Rodent cocktail (ab110413, USA), Complex IV, AMPK γ1, PGC-1α and Pparγ, FOXO1, TNF-α, IL-6 (USA, abCAM) and then incubated with polyclonal Goat anti-mouse IgG H&R (1:2000, ab6789, USA) in blocking buffer for 2 h at room temperature. To assure equal loading and/or to normalize protein content, the membrane blots were incubated with mouse anti-β-actin monoclonal antibody (mAbcam8227). Proteins were visualized using a chemiluminescence ECL Western blotting detection reagent (Bio-rad, USA). Quantification of protein was performed by chemiluminescence blot scanning (Quantity One™ version 4.6.3. Bio Rad).

Gene expression & RT qPCR Analyses. Total RNA was isolated using a commercially available kit (OMEGA bio-tek, R6812-02) according to the manufacturer's instructions. cDNA was generated using the PrimeScript RT reagent kit (TAKARA, RR047A). Gene expression were determined by qPCR as described in the product manual. The analysis was evaluated by Bio-Rad Real-Time System (CFX Connect™).

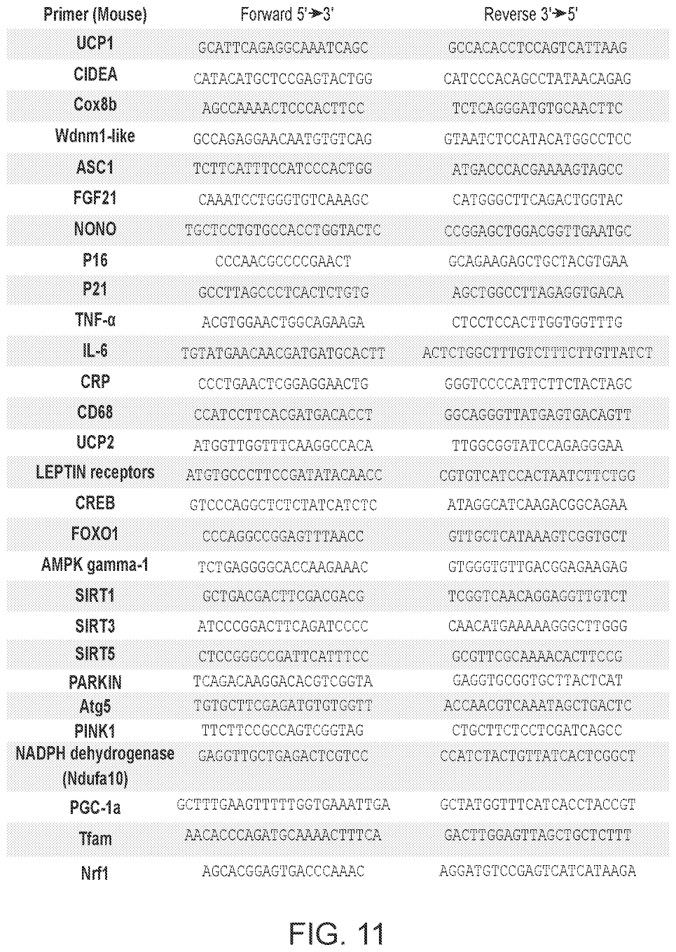

RNAs from 10 mice (5 treated and 5 Ctrl.) VF fat or livers were isolated by using omega Bio-Tek reagents, and cDNA was transcribed by (TAKARA). qPCR was then performed by using iQ SYBR Green mastermix bioscience on a CFX96 Real-Time System/C1000 Thermal Cycler (Bio-Rad). Gene expression was normalized to internal control NONO (mouse) or GAPdh (mouse). qPCR primers used are shown in FIG. 11.

Software code and data availability and Statistical Analyses. Statistical analyses were performed using Prism version 7.0 software (GraphPad). The data are presented as the mean±SEM. Two-tailed unpaired Student's t-test was used for 2-group treated and controls comparisons. One-way ANOVA and “Dunnett's multiple comparisons test” were used for treated and control specific gene expression. Two-way repeated-measures ANOVA and “Sidak's multiple comparisons test” were used to assess the effects of treated, control, multiple gene expressions and effects of energy homeostasis status, among old and young, treated and control mice samples. Pearson correlation coefficients were calculated as indicated. Data are expressed as mean±SEM n=3-10 and significance was considered at P values <0.05.

### Example 3: In Vivo Animal Study of Compound HG1a-1 on Cervical Cancer Mice Models

To investigate the effects of an example compound on a cervical cancer mouse model, HeLa cells were introduced to the back of mice (five groups, 5-6 mice in each group). After the tumor volume reached about 200 mm³, compound HG1a-1 solution (5, 20, 50 mg/kg) was dosed by intraperitoneal (IP) injection, every second day for ten days. Control mice were dosed with Saline and Taxol solution (PTX, 20 mg/kg). Tumor weight and volume were measured.

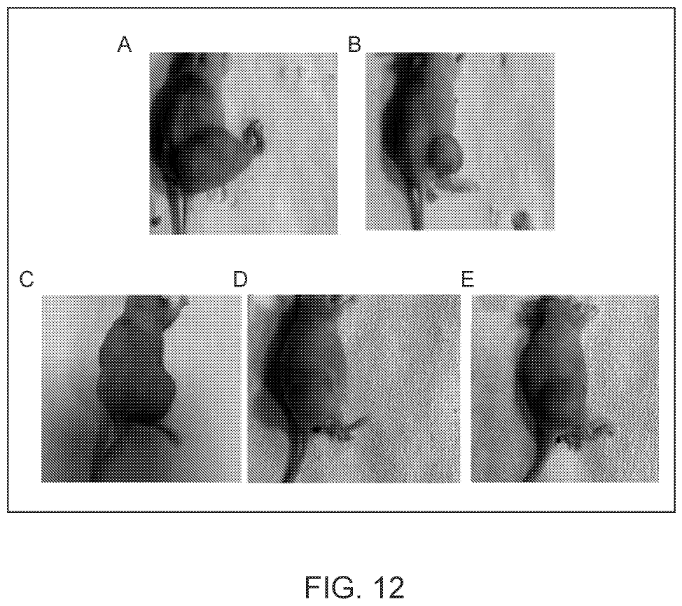

FIG. 12, panels A-E depicts images of the in vivo cervical cancer mouse study. Panel A depicts a saline control mouse. Panel B depicts a Taxol control mouse. Panel C-E depict mice dosed with 5 mg/kg, 20 mg/kg and 50 mg/kg of compound HG1a-1 respectively. As seen in Panels C-E, as the dose of HG1a-1 was increased, the tumor size decreased.

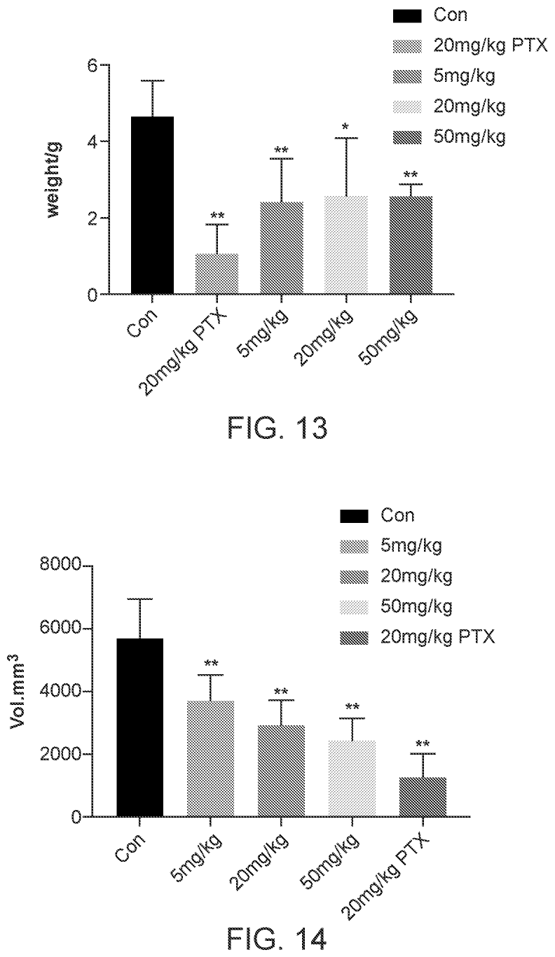

FIG. 13, illustrates the tumor weight measured in the in vivo cervical cancer mouse study after 10 days of administering compound HG1a-1. As shown in FIG. 13, at all dosages of 5, 20 and 50 mg/kg of compound HG1a-1, the tumor weight is decreased relative to the saline control (Con).

FIG. 14, illustrates the tumor volume measured in the in vivo cervical cancer mouse study after 10 days of administering compound HG1a-1. As shown in FIG. 14, at all dosages of 5, 20 and 50 mg/kg of compound HG1a-1, the tumor volume decreased, relative to the saline control (Con).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A compound of formula (I):
HG-L-X (I)
wherein:
HG is headgroup selected from a heterocyclic group, a heteroaryl group, and a guanidine, wherein the head group is optionally substituted;
L is a linker; and
X is a charged group,
Provided that the compound is not:

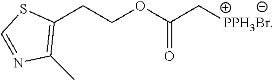

2. The compound of claim 1, wherein the headgroup is selected from a thiazole, a pyrazole, a thiophene, an oxazole, an oxadiazole, a tetrazole, a triazole, a pyridine, a pyrimidine, a pyrazine, a pyrazine, a triazine, a pyran, an oxazine, a thiazine a morpholine, a thiomorpholine, a piperidine and a piperazine.

3. The compound of claim 1, wherein the headgroup is any one of formula (HG1)-(HG9): 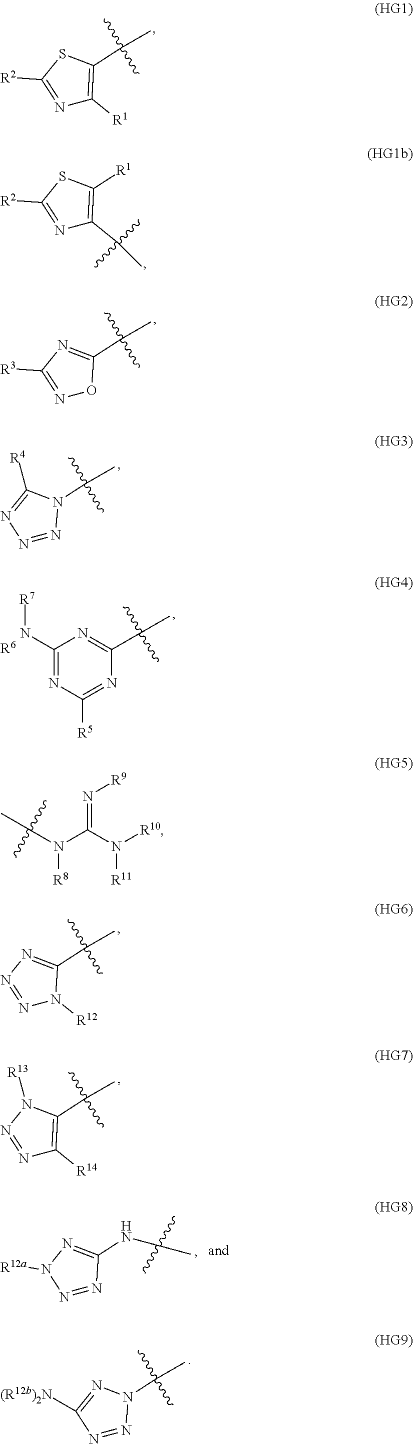
wherein:
R¹-R¹⁴ are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

4. The compound of claim 1, wherein the linker is described by the formula (L1): 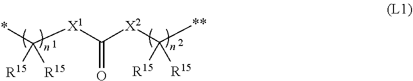
wherein:
* represents the point of connection to HG;
** represents the point of connection to X;
X¹ and X² are each independently selected from C(R¹⁵)₂, C(R¹⁵)₂(OCH₂CH₂O)ₙ₃, O, S and NR¹⁶;
each R¹⁵ is independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;
R¹⁶ is selected from hydrogen, alkyl, substituted alkyl, aryl, substituted aryl, carboxyl, acyl, substituted acyl, amino, substituted amino and hydroxyl;
n¹ an integer from 0 to 10;
n² is an integer from 0 to 10; and
n³ is an integer from 1 to 20.

5. The compound of claim 1 of the formula (IA) or (IB): 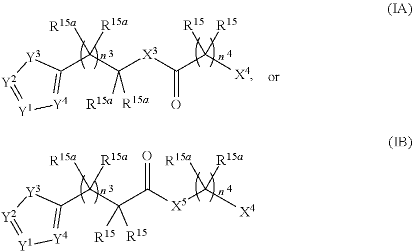
wherein:
Y¹, Y² and Y⁴ are each independently selected from N and CR¹⁵; Y³ is selected from S, O, NR¹⁶, and C(R¹⁵)₂;
X³ and X⁵ are each independently selected from C(R¹⁵)₂, O, S and NR¹⁶;
each R¹⁵ and R^{15a} are independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;
each R¹⁶ is independently selected from hydrogen, alkyl, substituted alkyl, aryl, substituted aryl, carboxyl, acyl, substituted acyl, amino, substituted amino, and hydroxyl;
X⁴ is a charged group;
n³ an integer from 0 to 10; and
n⁴ is an integer from 1 to 10.

6. The compound of claim 5 of the formula (IC) or (ID): 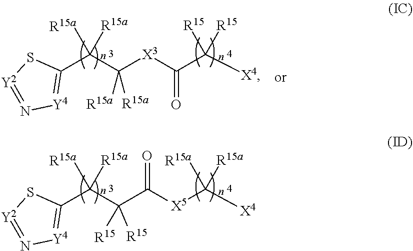
wherein:
Y² and Y⁴ are each CR¹⁵;
X³ and X⁵ are each independently selected from CR¹⁵, O, S and NR¹⁶;
each R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;
R¹⁶ is selected from hydrogen, alkyl, substituted alkyl, aryl, substituted aryl, carboxyl, acyl, substituted acyl, amino, substituted amino, and hydroxyl;
X⁴ is a charged group;
n³ an integer from 0 to 10; and
n⁴ is an integer from 1 to 10.

7. The compound of claim 6 of the formula (IE): 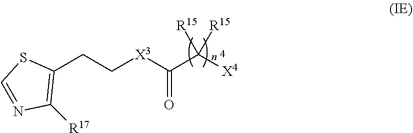
wherein:
X³ is selected from C(R¹⁵)₂, O, S and NR¹⁶;
each R¹⁵, and R¹⁷ are independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;
R¹⁶ is selected from hydrogen, alkyl, substituted alkyl, aryl, substituted aryl, carboxyl, acyl, substituted acyl, amino, substituted amino, and hydroxyl;
X⁴ is a charged group; and
n⁴ is an integer from 1 to 10.

8. The compound of claim 1, wherein the charged group is selected from a phosphonium cation, an ammonium cation, a quaternary ammonium cation, a pyridinium cation, an imidazolium cation, a guanidine moiety and an arginine moiety.

9. The compound of claim 1, described by a structure in any one of Table 1 to Table 8.

10. A method of treating a subject having a metabolic syndrome-related disease or a symptom thereof, the method comprising:
administering to the subject a therapeutically effective amount of a compound of the formula:
HG-L-X (I)
wherein:
HG is headgroup selected from a heterocyclic group, a heteroaryl group, and a guanidine group, wherein the head group is optionally substituted;
L is a linker; and
X is a charged group.

11. The method of claim 10, wherein the disease is selected from hyperlipidemia, type 2 diabetes, fatty liver disease, obesity, cardiovascular disease and stroke.

12. The method of claim 10, wherein the symptom is selected from abdominal obesity, insulin resistance, hyperinsulinemia, high levels of blood fats, increased blood pressure, and elevated serum lipids.

13. The method of claim 10, wherein the headgroup is selected from a thiazole, a pyrazole, a thiophene, an oxazole, an oxadiazole, a tetrazole, a triazole, a pyridine, a pyrimidine, a pyrazine, a pyrazine, a triazine, a pyran, an oxazine, a thiazine a morpholine, a thiomorpholine, a piperidine and a piperazine.

14. The method of claim 10, wherein the headgroup is any one of formula (HG1)-(HG9): 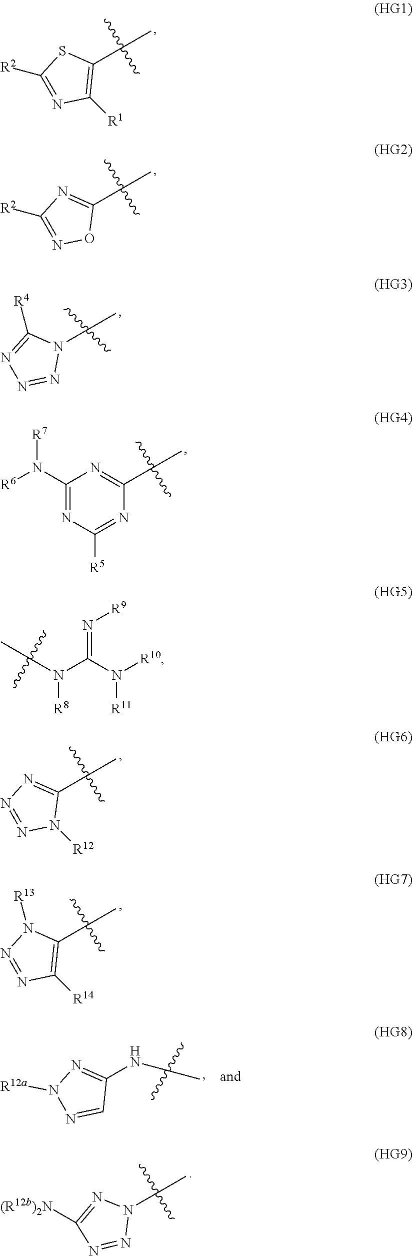
wherein:
R¹-R¹⁴ are each independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, and halogen.

15. The method of claim 10, wherein the linker is described by the formula (L1): 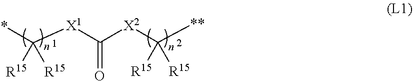
* represents the point of connection to HG;
** represents the point of connection to X;
X¹ and X² are each independently selected from C(R¹⁵)₂, C(R¹⁵)₂(OCH₂CH₂O)ₙ₃, O, S and NR¹⁶;
each R¹⁵ is independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;
R¹⁶ is selected from hydrogen, alkyl, substituted alkyl, aryl, substituted aryl, carboxyl, acyl, substituted acyl, amino, substituted amino and hydroxyl;
n¹ an integer from 0 to 10;
n² is an integer from 0 to 10; and
n³ is an integer from 1 to 20.

16. The method of claim 10, wherein the compound is of the formula (IA) or (IB): 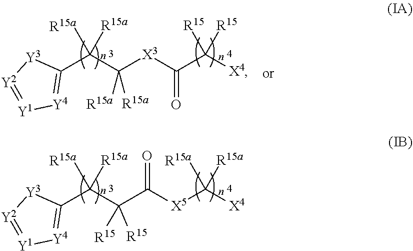
wherein:
Y¹, Y² and Y⁴ are each independently selected from N and CR¹⁵; Y³ is selected from S, O, NR¹⁶, and C(R¹⁵)₂;
X³ and X⁵ are each independently selected from C(R¹⁵)₂, O, S and NR¹⁶;
each R¹⁵ and R^{15a} are independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;
each R¹⁶ is independently selected from hydrogen, alkyl, substituted alkyl, aryl, substituted aryl, carboxyl, acyl, substituted acyl, amino, substituted amino, and hydroxyl;
X⁴ is a charged group;
n³ an integer from 0 to 10; and
n⁴ is an integer from 1 to 10.

17. The method of claim 16, wherein the compound is of the formula (IC) or (ID): 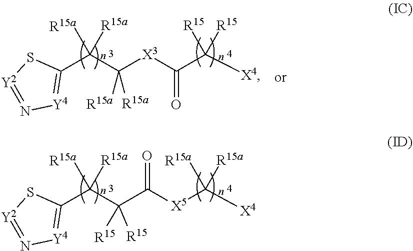
wherein:
Y² and Y⁴ are each CR¹⁵;
X³ and X⁵ are each independently selected from C(R¹⁵)₂, O, S and NR¹⁶;
each R¹⁵ and R^{15a} are each independently selected from hydrogen, alkyl, substituted alkyl, aryl, substituted aryl, amino, substituted amino, carboxyl, substituted carboxyl, acyl, substituted acyl, carboxamide, substituted carboxyamide, thiol, substituted thiol, alkoxy, substituted alkoxy, hydroxyl, and halogen;
R¹⁶ is selected from hydrogen, alkyl, substituted alkyl, aryl, substituted aryl, carboxyl, acyl, substituted acyl, amino, substituted amino, and hydroxyl;
X⁴ is a charged group;
n³ an integer from 0 to 10; and
n⁴ is an integer from 1 to 10.

18. The method of claim 17, wherein the compound is of the formula (IE): 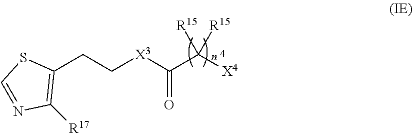
wherein:
X³ is selected from C(R¹⁵)₂, O, S and NR¹⁶;
each R¹⁵, and R¹⁷ are independently selected from hydrogen, alkyl, substituted alkyl, aryl, substituted aryl, amino, substituted amino, carboxyl, substituted carboxyl, acyl, substituted acyl, carboxamide, substituted carboxamide, thiol, substituted thiol, alkoxy, substituted alkoxy, hydroxyl, and halogen;
R¹⁶ is selected from hydrogen, alkyl, substituted alkyl, aryl, substituted aryl, carboxyl, acyl, substituted acyl, amino, substituted amino, and hydroxyl;
X⁴ is a charged group; and
n⁴ is an integer from 1 to 10.

19. The method of claim 10, wherein the charged group is selected from a phosphonium cation, an ammonium cation, a quaternary ammonium cation, a pyridinium cation, an imidazolium cation, a guanidine moiety, and an arginine moiety.

20. The method of claim 10, described by a structure in any one of Table 1 to Table 8.


SEQUENCE LISTING (56 sequences)

SEQ ID NO: 1 - DNA, 20 bp, Mus musculus
gcattcagaggcaaatcagc

SEQ ID NO: 2 - DNA, 21 bp, Mus musculus
catacatgctccgagtactgg

SEQ ID NO: 3 - DNA, 19 bp, Mus musculus
agccaaaactcccacttcc

SEQ ID NO: 4 - DNA, 21 bp, Mus musculus
gccagaggaacaatgtgtcag

SEQ ID NO: 5 - DNA, 21 bp, Mus musculus
tcttcatttccatcccactgg

SEQ ID NO: 6 - DNA, 20 bp, Mus musculus
caaatcctgggtgtcaaagc

SEQ ID NO: 7 - DNA, 23 bp, Mus musculus
tgctcctgtgccacctggtactc

SEQ ID NO: 8 - DNA, 16 bp, Mus musculus
cccaacgccccgaact

SEQ ID NO: 9 - DNA, 20 bp, Mus musculus
gccttagccctcactctgtg

SEQ ID NO: 10 - DNA, 19 bp, Mus musculus
acgtggaactggcagaaga

SEQ ID NO: 11 - DNA, 24 bp, Mus musculus
tgtatgaacaacgatgatgcactt

SEQ ID NO: 12 - DNA, 20 bp, Mus musculus
ccctgaactcggaggaactg

SEQ ID NO: 13 - DNA, 21 bp, Mus musculus
ccatccttcacgatgacacct

SEQ ID NO: 14 - DNA, 21 bp, Mus musculus
atggttggtttcaaggccaca

SEQ ID NO: 15 - DNA, 23 bp, Mus musculus
atgtgcccttccgatatacaacc

SEQ ID NO: 16 - DNA, 22 bp, Mus musculus
gtcccaggctctctatcatctc

SEQ ID NO: 17 - DNA, 19 bp, Mus musculus
cccaggccggagtttaacc

SEQ ID NO: 18 - DNA, 20 bp, Mus musculus
tctgaggggcaccaagaaac

SEQ ID NO: 19 - DNA, 19 bp, Mus musculus
gctgacgacttcgacgacg

SEQ ID NO: 20 - DNA, 20 bp, Mus musculus
atcccggacttcagatcccc

SEQ ID NO: 21 - DNA, 20 bp, Mus musculus
ctccgggccgattcatttcc

SEQ ID NO: 22 - DNA, 21 bp, Mus musculus
tcagacaaggacacgtcggta

SEQ ID NO: 23 - DNA, 21 bp, Mus musculus
tgtgcttcgagatgtgtggtt

SEQ ID NO: 24 - DNA, 19 bp, Mus musculus
ttcttccgccagtcggtag

SEQ ID NO: 25 - DNA, 20 bp, Mus musculus
gaggttgctgagactcgtcc

SEQ ID NO: 26 - DNA, 25 bp, Mus musculus
gctttgaagtttttggtgaaattga

SEQ ID NO: 27 - DNA, 23 bp, Mus musculus
aacacccagatgcaaaactttca

SEQ ID NO: 28 - DNA, 19 bp, Mus musculus
agcacggagtgacccaaac

SEQ ID NO: 29 - DNA, 21 bp, Mus musculus
gccacacctccagtcattaag

SEQ ID NO: 30 - DNA, 22 bp, Mus musculus
catcccacagcctataacagag

SEQ ID NO: 31 - DNA, 20 bp, Mus musculus
tctcagggatgtgcaacttc

SEQ ID NO: 32 - DNA, 22 bp, Mus musculus
gtaatctccatacatggcctcc

SEQ ID NO: 33 - DNA, 20 bp, Mus musculus
atgacccacgaaaagtagcc

SEQ ID NO: 34 - DNA, 20 bp, Mus musculus
catgggcttcagactggtac

SEQ ID NO: 35 - DNA, 22 bp, Mus musculus
ccggagctggacggttgaatgc

SEQ ID NO: 36 - DNA, 21 bp, Mus musculus
gcagaagagctgctacgtgaa

SEQ ID NO: 37 - DNA, 20 bp, Mus musculus
agctggccttagaggtgaca

SEQ ID NO: 38 - DNA, 20 bp, Mus musculus
ctcctccacttggtggtttg

SEQ ID NO: 39 - DNA, 27 bp, Mus musculus
actctggctttgtctttcttgttatct

SEQ ID NO: 40 - DNA, 22 bp, Mus musculus
gggtccccattcttctactagc

SEQ ID NO: 41 - DNA, 22 bp, Mus musculus
ggcagggttatgagtgacagtt

SEQ ID NO: 42 - DNA, 20 bp, Mus musculus
ttggcggtatccagagggaa

SEQ ID NO: 43 - DNA, 23 bp, Mus musculus
cgtgtcatccactaatcttctgg

SEQ ID NO: 44 - DNA, 21 bp, Mus musculus
ataggcatcaagacggcagaa

SEQ ID NO: 45 - DNA, 21 bp, Mus musculus
gttgctcataaagtcggtgct

SEQ ID NO: 46 - DNA, 21 bp, Mus musculus
gtgggtgttgacggagaagag

SEQ ID NO: 47 - DNA, 21 bp, Mus musculus
tcggtcaacaggaggttgtct

SEQ ID NO: 48 - DNA, 21 bp, Mus musculus
caacatgaaaaagggcttggg

SEQ ID NO: 49 - DNA, 20 bp, Mus musculus
gcgttcgcaaaacacttccg

SEQ ID NO: 50 - DNA, 20 bp, Mus musculus
gaggtgcggtgcttactcat

SEQ ID NO: 51 - DNA, 22 bp, Mus musculus
accaacgtcaaatagctgactc

SEQ ID NO: 52 - DNA, 20 bp, Mus musculus
ctgcttctcctcgatcagcc

SEQ ID NO: 53 - DNA, 23 bp, Mus musculus
ccatctactgttatcactcggct

SEQ ID NO: 54 - DNA, 23 bp, Mus musculus
gctatggtttcatcacctaccgt

SEQ ID NO: 55 - DNA, 22 bp, Mus musculus
gacttggagttagctgctcttt

SEQ ID NO: 56 - DNA, 23 bp, Mus musculus
aggatgtccgagtcatcataaga